(19) United States
(12) Patent Application Publication
(10) Pub. No.: US 2023/0149493 A1
(43) Pub. Date: May 18, 2023

(54) COMPOSITIONS AND METHODS FOR TREATING COVID-19 AND SYMPTOMS THEREOF

(71) Applicant: YUVA BIOSCIENCES, INC., Birmingham, AL (US)

(72) Inventors: Keshav K. Singh, Birmingham, AL (US); Leopoldo Aguilera Aguirre, Birmingham, AL (US)

(21) Appl. No.: 17/806,014

(22) Filed: Jun. 8, 2022

Related U.S. Application Data
(60) Provisional application No. 63/208,470, filed on Jun. 8, 2021.

(51) Int. Cl. A61K 36/61; A61K 36/03; A61K 36/185; A61P 31/12

(52) U.S. Cl. CPC A61K 36/61; A61K 36/03; A61K 36/185; A61P 31/12

(57) ABSTRACT

Methods for treating or preventing viral infection-induced symptoms are disclosed. Methods for treating or preventing a viral infection are disclosed. Methods for treating or preventing mitochondrial dysfunction are disclosed. The viral infections include SARS-CoV-2, HIV, Influenza, and MERS. The methods include administering an emblica extract or a compound constituent of or having a similarity score of at least 95% with a compound constituent of an emblica extract, a fucus extract or a compound constituent of or having a similarity score of at least 95% with a compound constituent of a fucus extract, or a chebula extract or a compound constituent of or having a similarity score of at least 95% with a compound constituent of a chebula extract.

### CROSS-REFERENCE TO RELATED APPLICATIONS

This application claims priority to U.S. Provisional Application Ser. No. 63/208,470, filed on Jun. 8, 2021, titled “COMPOSITIONS AND METHODS FOR TREATING COVID-19 AND SYMPTOMS THEREOF,” which is incorporated herein by reference in its entirety for all purposes.

### FIELD OF TECHNOLOGY

Aspects and embodiments disclosed herein relate to methods of treatment of diseases and conditions related to mitochondrial dysfunction. More specifically, aspects and embodiments disclosed herein relate to methods of treatment of diseases and conditions related to mitochondrial DNA depletion.

### BACKGROUND

Mitochondrial dysfunction, such as mitochondrial DNA (mtDNA) depletion, is involved in many diseases and conditions, such as mtDNA depletion syndromes, mitochondrial diseases, viral infections, viral infection-induced symptoms, aging, aging-associated chronic diseases, reduced energy levels and vitality, and other human pathologies. Fundamental questions about mitochondrial biology and mtDNA biology and their roles in such diseases and conditions remain mostly unsolved. Animal models capable of inducing mitochondrial dysfunction and/or modulating mtDNA copy number and/or concentration have been developed and provide a system for investigating mitochondrial pathology and its role in the disease process. There is a need for treatment methods for diseases and conditions related to mitochondrial dysfunction, such as mtDNA depletion.

### SUMMARY

In accordance with one aspect, there is provided a method of treating or preventing viral infection-induced symptoms in a subject. The method may comprise administering to the subject a composition comprising an effective amount of one or more of an emblica extract, a fucus extract, and a chebula extract, or a pharmaceutically acceptable form thereof, or one or more compounds constituent of one or more of an emblica extract, a fucus extract, and a chebula extract or a pharmaceutically acceptable form thereof, or one or more compounds having a similarity score of at least 95% with a compound constituent of one or more of an emblica extract, a fucus extract, and a chebula extract, or a pharmaceutically acceptable form thereof.

In accordance with another aspect, there is provided a method of treating or preventing a viral infection in a subject. The method may comprise administering to the subject a composition comprising an effective amount of one or more of an emblica extract, a fucus extract, and a chebula extract, or a pharmaceutically acceptable form thereof, or one or more compounds constituent of one or more of an emblica extract, a fucus extract, and a chebula extract, or a pharmaceutically acceptable form thereof, or one or more compounds having a similarity score of at least 95% with a compound constituent of one or more of an emblica extract, a fucus extract, and a chebula extract, or a pharmaceutically acceptable form thereof.

In accordance with another aspect, there is provided a method of treating or preventing mitochondrial dysfunction in a subject. The method may comprise administering to the subject a composition comprising an effective amount of one or more of an emblica extract, a fucus extract, or a chebula extract, or a pharmaceutically acceptable form thereof, or one or more compounds constituent of one or more of an emblica extract, a fucus extract, and a chebula extract, or a pharmaceutically acceptable form thereof, or one or more compounds having a similarity score of at least 95% with a compound constituent of one or more of an emblica extract, a fucus extract, and a chebula extract, or a pharmaceutically acceptable form thereof.

The method may comprise administering to the subject an effective amount of an emblica extract one or more compound constituent of an emblica extract or having a similarity score of at least 95% with a compound constituent of an emblica extract, or a pharmaceutically acceptable form thereof.

The method may comprise administering to the subject an effective amount of a fucus extract one or more compound constituent of a fucus extract or having a similarity score of at least 95% with a compound constituent of a fucus extract, or a pharmaceutically acceptable form thereof.

The method may comprise administering to the subject an effective amount of a chebula extract, one or more compound constituent of a chebula extract or having a similarity score of at least 95% with a compound constituent of a chebula extract, or a pharmaceutically acceptable form thereof.

In some embodiments, the emblica extract is derived from Emblica officinalis.

In some embodiments, the compound constituent of the emblica extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a-C(O)OH, wherein a is 0 to 5, and 1 to 5 hydroxy groups, or a pharmaceutically acceptable form thereof, or a combination of the foregoing.

In some embodiments, the compound constituent of the emblica extract is gallic acid, vanillic acid, chlorogenic acid, 5 caffeic acid, syringic acid, coumaric acid, quercetin, emblicanin A, emblicanin B, punigluconin, and pedunculagin, punicafolin, phyllanemblin, kaempferol, ellagic acid, chebulinic acid, chebulagic acid, punicalagin, a metabolite of any of the foregoing, a compound having a similarity score of at least 95% with a metabolite of any of the foregoing, or a pharmaceutically acceptable form of any of the foregoing.

In some embodiments, the fucus extract is derived from Fucus vesiculosus, Fucus serratus, Fucus, spiralis, or Fucus guiryi.

In some embodiments, the compound constituent of the fucus extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a-C(O)OH, wherein a is 0 to 5, and 1 to 5 hydroxy groups, or a pharmaceutically acceptable form thereof, or a combination of the foregoing.

In some embodiments, the compound constituent of the fucus extract is gallic acid, vanillic acid, chlorogenic acid, caffeic acid, syringic acid, coumaric acid, quercetin, fucoidan, punigluconin, and pedunculagin, punicafolin, phyllanemblin, kaempferol, ellagic acid, chebulinic acid, chebulagic acid, punicalagin, a metabolite of any of the foregoing, a compound having a similarity score of at least 95% with a metabolite of any of the foregoing, or a pharmaceutically acceptable form of any of the foregoing.

In some embodiments, the chebula extract is derived from Terminilia chebula, Terminalia arborea, or Lumnitzera racemose.

In some embodiments, the compound constituent of the chebula extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a-C(O)OH, wherein a is 0 to 5, and 1 to 5 hydroxy groups, or a pharmaceutically acceptable form thereof, or a combination of the foregoing.

In some embodiments, the compound constituent of the chebula extract is gallic acid, vanillic acid, chlorogenic acid, caffeic acid, syringic acid, coumaric acid, quercetin, fucoidan, punigluconin, and pedunculagin, punicafolin, phyllanemblin, kaempferol, ellagic acid, chebulinic acid, chebulagic acid, punicalagin, a metabolite of any of the foregoing, a compound having a similarity score of at least 95% with a metabolite of any of the foregoing, or a pharmaceutically acceptable form of any of the foregoing.

In some embodiments, the composition comprises two or more of: an effective amount of an emblica extract, or a pharmaceutically acceptable form thereof, or a compound constituent of an emblica extract, or a pharmaceutically acceptable form thereof, or a compound having a similarity score of at least 95% with a compound constituent of an emblica extract, or a pharmaceutically acceptable form thereof; an effective amount of a fucus extract, or a pharmaceutically acceptable form thereof, or a compound constituent of fucus extract, or a pharmaceutically acceptable form thereof, or a compound having a similarity score of at least 95% with a compound constituent of a fucus extract, or a pharmaceutically acceptable form thereof; and an effective amount of a chebula extract, or a pharmaceutically acceptable form thereof, or a compound constituent of a chebula extract, or a pharmaceutically acceptable form thereof, or a compound having a similarity score of at least 95% with a compound constituent of a chebula extract, or a pharmaceutically acceptable form thereof.

In some embodiments, the composition is fortified with one or more compounds constituent of an emblica extract, or a pharmaceutically acceptable form thereof, or a compound having a similarity score of at least 95% with a compound constituent of an emblica extract, or a pharmaceutically acceptable form thereof.

In some embodiments, the composition is fortified with one or more compounds constituent of a fucus extract, or a pharmaceutically acceptable form thereof, or a compound having a similarity score of at least 95% with a compound constituent of a fucus extract, or a pharmaceutically acceptable form thereof.

In some embodiments, the composition is fortified with one or more compounds constituent of a chebula extract, or a pharmaceutically acceptable form thereof, or a compound having a similarity score of at least 95% with a compound constituent of a chebula extract, or a pharmaceutically acceptable form thereof.

In some embodiments, the one or more compounds constituent of the emblica extract or having a similarity score of at least 95% with a compound constituent of the emblica extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

In some embodiments, the one or more compounds constituent of the fucus extract or having a similarity score of at least 95% with a compound constituent of the fucus extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

In some embodiments, the one or more compounds constituent of the chebula extract or having a similarity score of at least 95% with a compound constituent of the chebula extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

In some embodiments, the viral infection-induced symptoms in the subject comprise one or more acute and/or chronic symptoms, e.g., muscle or body aches, fatigue, shortness of breath, difficulty breathing, fever or chills, headache, loss of taste or smell, sore throat, congestion or runny nose, nausea or vomiting, diarrhea, cough, lymphadenitis, rash, or sleep hyperhidrosis.

In some embodiments, the viral infection in the subject is SARS, e.g., SARS-CoV-1 or SARS-CoV-2.

In some embodiments, the viral infection in the subject is HIV.

In some embodiments, the viral infection in the subject is Influenza.

In some embodiments, the viral infection in the subject is MERS.

In some embodiments, the viral infection in the subject is any viral infection related to mitochondrial dysfunction in the subject.

In some embodiments, the effective amount is a therapeutically effective amount.

In some embodiments, the treatment or prevention involves inducing mitochondrial biogenesis and/or improving mitochondrial function.

In some embodiments, the effective amount or therapeutically effective amount is sufficient to induce mitochondrial biogenesis.

In some embodiments, administration increases expression of at least one protein selected from PGC-1a, TFAM, NRF-1, and COX II.

In some embodiments, administration decreases expression or inhibits an increase of expression of at least one protein selected from FGF-21 and IL-6, or any cytokine related to viral infection.

In some embodiments, administration increases or inhibits a decrease in at least one of ATP-linked respiration, maximal respiration and reserve capacity in subjects infected with SARS-CoV-2.

In some embodiments, administration decreases the release of viral vesicles from dysfunctional mitochondria.

In some embodiments, administration modulates, e.g., increases or decreases, viral protein interaction with one or more host mitochondrial genes, e.g., MRPS2, MRPS5, MRPS25, MRPS27, NDUFAF1, NDUFB9, NDUFAF2, ATP1B1, ATP6V1A, ACADM, AASS, PMPCB, PITRM1, COQ8B, PMPCA, and Tomm70.

In some embodiments, administration decreases or inhibits an increase in circulating mtDNA levels, e.g., plasma mtDNA and cytoplasmic mtDNA.

In some embodiments, the composition is administered topically.

In some embodiments, the composition is administered parenterally, e.g., intravenously, intraperitoneally, or intramuscularly.

In some embodiments, the composition is administered enterally.

In some embodiments, the composition is formulated as a topical solution, oil, cream, emulsion, or gel.

In some embodiments, the composition is formulated as a shampoo, conditioner, spray, cream, gel, balm, body wash, soap, lotion, or make-up.

In some embodiments, the composition is formulated as a parenteral liquid solution.

In some embodiments, the composition is formulated as an enteral capsule or tablet, or dietary supplement or food, e.g., food, food supplement, medical food, food additive, nutraceutical, or drink.

In some embodiments, the composition is administered locally.

In some embodiments, the composition is administered systemically.

In some embodiments, the composition is formulated for immediate release.

In some embodiments, the composition is formulated for extended release, e.g., controlled or sustained release.

In some embodiments, the composition is administered in combination with standard of care treatment for one or more of SARS-CoV-2, HIV, SARS-CoV-2, HIV, Influenza, MERS, or any viral infection related to mitochondrial dysfunction in a subject.

In some embodiments, the composition is administered in combination with one or more drugs for symptomatic relief, e.g., acetaminophen, ibuprofen, bismuth subsalicylate, loperamide, oxymetazoline, phylephrine, psudoephedrine, or hydrocortisone.

In some embodiments, the composition is administered in combination with one or more anti-viral drugs, e.g., remdesivir, abacavir, didanosine, emtricitabine, iamivudine, stavudine, zalcitabine, zidovudine, tenofovir disproval fumarate, peramivir, zanamivir, oseltamivir phosphate, baloxavir marboxil, ribavirin, interferon-α, lopinavir/ritonavir, and convalescent plasma.

In some embodiments, the composition comprises a nanoparticle-based delivery carrier.

In some embodiments, the composition comprises a skin penetration enhancer or is administered in combination with a skin penetration enhancer, e.g., a chemical skin penetration enhancer or a physical skin penetration enhancer.

In some embodiments, the composition comprises a mitochondria-targeting agent or a delivery carrier functionalized with a mitochondria-targeting agent.

In some embodiments, the compound constituent of an emblica extract was derived from, purified from, or isolated from the emblica extract.

In some embodiments, the compound constituent of an emblica extract was derived from, purified from, or isolated from a source other than the emblica extract.

In some embodiments, the compound constituent of an emblica extract was synthesized.

In some embodiments, compound constituent of a fucus extract was derived from, purified from, or isolated from the fucus extract.

In some embodiments, the compound constituent of a fucus extract was derived from, purified from, or isolated from a source other than the fucus extract.

In some embodiments, the compound constituent of a fucus extract was synthesized.

In some embodiments, the compound constituent of a chebula extract was derived from, purified from, or isolated from the chebula extract.

In some embodiments, the compound constituent of a chebula extract was derived from, purified from, or isolated from a source other than the chebula extract.

In some embodiments, the compound constituent of a chebula extract was synthesized.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A shows quantification of immunofluorescence analysis of OXPHOS complex IV (COXII) in relative fluorescence units (RFUs) in paraffin dorsal skin sections control and mtDNA-depleter mice after dox induction and 16 weeks of topical emblica extract treatment or control treatment. Data are mean±SD (n=3); *P<0.05;

FIG. 1B shows RT-PCR analysis of dorsal skin RNA for mtDNA encoded genes from control and mtDNA-depleter mice after dox induction and 16 weeks of topical emblica extract treatment or control treatment;

FIG. 1C shows quantification of mtDNA content (mean±s.e.m; *P<0.05, Student's t test) in dorsal skin samples from control (n=3) and mtDNA-depleter (n=3) mice after dox induction and 16 weeks of topical emblica extract treatment or control treatment application;

FIG. 1D shows quantitative analysis of inflammatory cells in the skin sections from control and mtDNA-depleter mice after dox induction and 16 weeks of topical emblica extract treatment or control treatment (mean±SD; *P≤0.05; **P≤30.01; ***P≤0.001 Student's t test);

FIG. 2A shows representative pictures of: (i) control mice after dox induction; (ii) mtDNA-depleter mice after dox-induction; (iii) control mice after dox induction and 51 days of treatment with fucus extract; and (iv) mtDNA-depleter mice after dox induction and 51 days of treatment with fucus extract (preventive intervention) (n=3 for each group);

FIG. 2B shows quantification of mtDNA content (mean±s.e.m; *P<0.05, Student's t test) in dorsal skin samples from control (n=3) and mtDNA-depleter (n=3) mice after dox induction and 16 weeks of topical fucus extract treatment or control treatment application;

FIG. 3 shows schematics of emblica extract preventive and therapeutic in vivo experiments;

FIG. 4A is a graph showing induced expression of mitochondrial complex IV subunit 2 (COXII) by various compositions, according to one embodiment;

FIG. 4B is a graph showing induced expression of mitochondrial transcription factor A (TFAM) by various compositions, according to one embodiment;

FIG. 4C is a graph showing induced expression of peroxisome proliferator-activated receptor gamma coactivator 1-alpha (PGC-1a) by various compositions, according to one embodiment;

FIG. 5 is an image of an electrophoresis gel showing protein levels of the mitochondrial biogenesis markers COXII, TFAM, and PGC-1a induced by various compositions, according to one embodiment;

FIG. 6 is a schematic diagram summarizing the mechanisms involved in SARS-CoV-2 hijacking of host mitochondria;

FIGS. 7A-7F are graphs showing cellular mitochondrial profile in human PBMCs of healthy controls (HC) (n=6), patients with PCR-positive COVID-19 (n=6), and patients with chest infection (negative for COVID-19; n=7);

FIGS. 8A-8D are graphs showing the results from Phenotype Stress Tests showing high basal and stressed glycolysis in peripheral blood mononuclear cells (PBMCs) from patients with COVID-19: live PBMCs from healthy controls (HC) (n=6), patients with PCR-positive COVID-19 (n=5), and patients with chest infection (Chest inf; negative for COVID-19; n=7);

FIGS. 9A-9C are graphs showing results from Substrate Oxidation Stress Tests interrogating three primary substrates that drive the mitochondrial activities: long-chain fatty acids (LCFAs), glucose/pyruvate, and/or glutamine in peripheral blood mononuclear cells (PBMCs) from patients with COVID-19: live PBMCs from patients with COVID-19 (n=5);

FIGS. 10A-10D are graphs showing circulating levels of fibroblast growth factor 21 (FGF-21), a mitokine, in healthy controls (HCs), patients with COVID-19, and patients with chest infection (Chest inf), and its correlation with mitochondrial functional parameters;

FIGS. 11A-11B are graphs showing circulating levels of interleukin-6 (IL-6) in healthy controls (HCs), patients with COVID-19, and patients with chest infection;

FIGS. 12A-12F are graphs showing COXII expression induced by administration of an emblica extract in vitro at 6 hours to 96 hours after administration;

FIGS. 13A-13F are graphs showing TFAM expression induced by administration of an emblica extract in vitro at 6 hours to 96 hours after administration;

FIGS. 14A-14F are graphs showing PCG-1a expression induced by administration of an emblica extract in vitro at 6 hours to 96 hours after administration;

FIGS. 15A-15D are graphs showing COXII expression induced by administration of an emblica extract in vitro, optionally with a nanoparticle delivery carrier, at 6 hours and 24 hours after administration;

FIGS. 16A-16B include graphs showing expression of COXII and TFAM at 6 hours after in vitro administration of a constituent of emblica extract, according to one embodiment;

FIGS. 17A-17D include graphs showing expression of COXII and TFAM at 12 hours and 24 hours after in vitro administration of a constituent of emblica extract, according to one embodiment;

FIGS. 18A-18D include graphs showing expression of COXII and TFAM at 12 hours and 24 hours after in vitro administration of a constituent of emblica extract, according to one embodiment;

FIGS. 19A-19D include graphs showing expression of COXII and TFAM at 12 hours and 24 hours after in vitro administration of a constituent of emblica extract, according to one embodiment;

FIGS. 20A-20D include graphs showing expression of COXII and TFAM at 12 hours and 24 hours after in vitro administration of a constituent of emblica extract, according to one embodiment;

FIGS. 21A-21D include graphs showing expression of COXII at 6, 12, 24, and 48 hours after in vitro administration of a constituent of emblica extract, according to one embodiment;

FIGS. 22A-22D include graphs showing expression of TFAM at 6, 12, 24, and 48 hours after in vitro administration of a constituent of emblica extract, according to one embodiment;

FIGS. 23A-23D include graphs showing expression of COXII at 6, 12, 24, and 48 hours after in vitro administration of a constituent of emblica extract, according to one embodiment;

FIGS. 24A-24D include graphs showing expression of TFAM at 6, 12, 24, and 48 hours after in vitro administration of a constituent of emblica extract, according to one embodiment;

FIGS. 25A-25D include graphs showing expression of COXII at 6, 12, 24, and 48 hours after in vitro administration of a constituent of emblica extract, according to one embodiment;

FIGS. 26A-26D include graphs showing expression of TFAM at 6, 12, 24, and 48 hours after in vitro administration of a constituent of emblica extract, according to one embodiment;

FIGS. 27A-27D include graphs showing expression of COXII at 6, 12, 24, and 48 hours after in vitro administration of a constituent of emblica extract, according to one embodiment;

FIGS. 28A-28D include graphs showing expression of TFAM at 6, 12, 24, and 48 hours after in vitro administration of a constituent of emblica extract, according to one embodiment;

FIGS. 29A-29D include graphs showing expression of COXII at 6, 12, 24, and 48 hours after in vitro administration of a chebulinic acid, optionally encapsulated in a nanoparticle carrier, according to one embodiment;

FIGS. 30A-30D include graphs showing expression of TFAM at 6, 12, 24, and 48 hours after in vitro administration of chebulinic acid, optionally encapsulated in a nanoparticle carrier, according to one embodiment;

FIGS. 31A-31B include graphs showing expression of COXII at 6 hours after in vitro administration of emblica extract encapsulated in a nanoparticle carrier, according to one embodiment;

FIGS. 32A-32D include graphs showing expression of COXII at 6, 12, 24, and 48 hours after in vitro administration of emblica extract encapsulated in a nanoparticle carrier, according to one embodiment;

FIGS. 33A-33D include graphs showing expression of TFAM at 6, 12, 24, and 48 hours after in vitro administration of emblica extract encapsulated in a nanoparticle carrier, according to one embodiment;

FIGS. 34A-34D include graphs showing expression of COXII at 6, 12, 24, and 48 hours after in vitro administration of emblica extract encapsulated in a nanoparticle carrier, according to one embodiment;

FIGS. 35A-35D include graphs showing expression of COXII at 6, 12, 24, and 48 hours after in vitro administration of emblica extract encapsulated in a nanoparticle carrier, according to one embodiment; and

FIGS. 36A-36D include graphs showing expression of TFAM at 6, 12, 24, and 48 hours after in vitro administration of emblica extract encapsulated in a nanoparticle carrier, according to one embodiment.

### DETAILED DESCRIPTION

Mitochondrial dysfunction is associated with many mitochondrial diseases, many of which are the result of dysfunctional mitochondrial oxidative phosphorylation (OXPHOS). Mitochondrial OXPHOS accounts for the generation of most of the cellular adenosine triphosphate (ATP) in a cell. The OXPHOS function largely depends on the coordinated expression of proteins encoded by both nuclear and mitochondrial genomes. The human mitochondrial genome encodes for 13 polypeptides of the OXPHOS system, and the nuclear genome encodes the remaining more than 85 polypeptides required for the assembly of OXPHOS system. mtDNA depletion impairs OXPHOS and leads to mtDNA depletion syndromes (Alberio, et al., Mitochondrion 7, 6-12, 2007; Ryan, M et al., Annu. Rev. Biochem. 76, 701-722, 2007). The mtDNA depletion syndromes are a heterogeneous group of disorders, characterized by low mtDNA levels in specific tissues. In different target organs, mtDNA depletion leads to specific pathological changes (Tuppen, et al., Biochim. Biophys. Acta 1797, 113-128, 201)). mtDNA depletion syndromes result from the genetic defects in the nuclear-encoded genes that participate in mtDNA replication, and mitochondrial nucleotide metabolism and nucleotide salvage pathway (Alberio, et al., Mitochondrion 7, 6-12, 2007). mtDNA depletion is also implicated in other human diseases and conditions, such as, but not limited to, mtDNA depletion syndromes, mitochondrial diseases, viral infections, viral infection-induced symptoms, aging, aging-associated chronic diseases or conditions, reduced energy levels and vitality, characteristics of hair aging including hair loss and graying, characteristics of skin aging including skin wrinkles and senile lentigines, skin diseases and conditions, and other human pathologies.

Viral infections and viral infection-induced symptoms may be exacerbated by aging. Advanced age individuals are often at greater risk of severe infection, severe symptoms, and even death as a result of a viral infection, such as a COVID-19 infection. A general decline in mitochondrial function has been extensively reported during aging. Furthermore, mitochondrial dysfunction is known to be a driving force underlying age-related human diseases. A mouse that carries a specific mtDNA mutation has been shown to present signs of premature aging (i.e., a mtDNA depleter mouse). In addition to mutations in mtDNA, studies also suggest a decrease in mtDNA content and mitochondrial copy number with age. Low mtDNA copy number is linked to frailty and, for a multiethnic population, is a predictor of all-cause mortality. A recent study revealed that humans on an average lose about four copies of mtDNA every ten years. This study also identified an association of decrease in mtDNA copy number with age-related physiological parameters.

Accumulating evidence suggests a strong link between mitochondrial dysfunction, mitochondrial diseases, aging, and aging-associated diseases. Notably, increased somatic mtDNA mutations and decline in mitochondrial functions have been extensively reported during human aging. Studies also suggest a decrease in mtDNA content and mitochondrial number with age.

### Definitions

As used herein, the term “carrier” refers to a diluent or vehicle with which a compound is administered. The carrier may be a pharmaceutically acceptable carrier. The carrier may be a cosmetically acceptable carrier. Examples of suitable pharmaceutical carriers are described in “Remington's Pharmaceutical Sciences” by E. W. Martin.

As used herein, the term “corresponding aspartic acid” means an aspartic acid (D) residue that is mutated to an alanine (A) residue in a POLG1 amino acid sequence that is the equivalent of the aspartic acid at position 1135 of the human POLG1 sequence. In a particular embodiment, the “corresponding aspartic acid” is flanked on the amino terminus side by an amino acid sequence of S/T I/V H X, I S/T I/V H X, or C/A I S/T I/V H X, and/or on the carboxy terminus side by an amino acid sequence of X E V/I R, X E V/I R Y/F, or X E V/I R Y/F L, wherein “X” indicates the aspartic acid amino acid that is mutated or will be mutated to alanine.

As used herein, the terms “depleted,” “depletion,” or “depleter” with respect to mtDNA refers to a decrease in mtDNA copy number and/or concentration in a human or in a non-human animal, tissue, or cell of the non-human animal. Such a determination may be made with regard to a human that has not been administered a compound or composition of the disclosure or to a control non-human animal tissue, or cell (for example, a non-human animal that has not expressed or does not express a mutant POLG1 polypeptide).

As used herein, “treatment” of a disease or condition refers to reducing or lessening the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein, the term “effective amount” refers to an amount of a compound or composition of the disclosure administered to a subject, which is effective to provide a desired response and/or effect in the subject. The response and/or effect may be a cosmetic response and/or effect. The response and/or effect may be a therapeutic response and/or effect. The response and/or effect may be a prophylactic or preventative response and/or effect.

As used herein, the term “therapeutically effective amount” refers to an amount of compound or composition of the disclosure administered to a subject, which is effective to treat a disease or condition described herein in a subject and/or to produce a desired physiological response and/or therapeutic effect in the subject. One example of a desired physiological response includes increasing mtDNA copy number and/or concentration.

The actual dose which comprises the “effective amount” or “therapeutically effective amount” may depend upon the route of administration, the size and health of the subject, the disorder being treated, and the like.

In some embodiments, the “effective amount” or “therapeutically effective amount” in the context of the present disclosure may be sufficient to treat or prevent viral infection-induced symptoms and/or treat or prevent viral infection.

In some embodiments, the “effective amount” or “therapeutically effective amount” in the context of the present disclosure is sufficient to induce mitochondrial biogenesis. The “effective amount” or “therapeutically effective amount” may be sufficient to induce mitochondrial biogenesis locally. The “effective amount” or “therapeutically effective amount” may be sufficient to induce mitochondrial biogenesis systemically.

In some embodiments, the “effective amount” or “therapeutically effective amount” in the context of the disclosure decreases an inflammatory phenotype, increases expression of mitochondrial oxidative phosphorylation complexes, increases stability of mitochondrial oxidative phosphorylation complexes, alters, e.g., decreases expression of at least one gene selected from the group consisting of: NF-κB, COX-2, INF-β1, CCL5, MMP1, MMP2, MMP9, MMP13, IGF1R, VEGF, and MRPS5, alters, e.g., increases expression of at least one gene selected from the group consisting of: TIMP1, KLOTHO, COL1A1, MTCO2, TFAM, and VDAC, prevents a deactivation of a gene associated with mitochondrial health and activity, selected from: FGF2, FGFR1, COX7A1, PDK4, FAM173A, MRPL12, and WNT11, decreases inflammatory infiltrate in the skin and/or hair follicles, and/or increases expression of at least one protein selected from PGC-1a, TFAM, NRF-1, and COXII.

In some embodiments, the “effective amount” or “therapeutically effective amount” in the context of the disclosure increases mtDNA copy number or concentration by at least 5%, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%. In each of the foregoing, when a reduction or increase is specified, such reduction or increase may be determined with respect to a subject or non-human animal that has not been treated with a compound or composition of the disclosure and that is suffering from a disease or condition described herein.

In some embodiments, the “effective amount” or “therapeutically effective amount” in the context of the disclosure prevents incidence of an inflammatory phenotype, prevents a decrease in expression of mitochondrial oxidative phosphorylation complexes, prevents a decrease in stability of mitochondrial oxidative phosphorylation complexes, prevents an increase in expression of at least one gene selected from the group consisting of: NF-κB, COX-2, INF-β1, CCL5, MMP1, MMP2, MMP9, MMP13, IGF1R, VEGF, and MRPS5, prevents a decrease in expression of at least one gene selected from the group consisting of: TIMP1, KLOTHO, COL1A1, MTCO2, TFAM, and VDAC, prevents a deactivation of a gene associated with mitochondrial health and activity, selected from: FGF2, FGFR1, COX7A1, PDK4, FAM173A, MRPL12, and WNT11, prevents an increase in inflammatory infiltrate in the skin and/or hair follicles, prevents a decrease in mtDNA copy number or concentration, and/or prevents a decrease in expression of at least one protein selected from PGC-1a, TFAM, NRF-1, and COXII.

As used herein, the term “excipient” means a substance formulated alongside the active ingredient of a composition included for purposes such as, but not limited to, long-term stabilization, bulking up solid formulations that contain potent active ingredients in small amounts (thus often referred to as bulking agents, fillers, or diluents), or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life. The excipient may be a pharmaceutically acceptable excipient. The excipient may be a cosmetically acceptable excipient. Examples of suitable excipients are described in “Remington's Pharmaceutical Sciences” by E. W. Martin.

As used herein, the term “in need of” (such as in the phrase “in need of treatment”) refers to a judgment made by a healthcare professional that a subject requires or will benefit from administration of a compound of the disclosure. This judgment is made based on a variety of factors that are in the realm of a healthcare professional's expertise, such as, but not limited to, the knowledge that the subject is ill, or will be ill, as the result of a disease or condition that is treatable by a method or drug composition of the disclosure.

As used herein, the terms “mutant POLG1” or “mutated POLG1” refers to a POLG1 amino acid sequence from a particular species that contains at least one mutation as compared to the wild-type POLG1 sequence from that species. A mutation need not cause a disease. A single mutation or more than one mutation may be present. In a particular embodiment, a single dominant negative mutation may be present (such as, but not limited to, a D1135A mutation), optionally with one or additional mutations.

As used herein, the term “pharmaceutically acceptable” refers to a compound that is compatible with the other ingredients of a composition and not deleterious to the subject receiving the compound or composition. In some embodiments, the term “pharmaceutically acceptable” means approved by a regulatory agency of the Federal or a state government or listed in the U.S. Pharmacopeia or other generally recognized pharmacopeia for use in animals, and more particularly in humans.

As used herein, the term “cosmetically acceptable” refers to a compound that is compatible with the other cosmetic ingredients of a composition and not deleterious to the subject receiving the compound or composition. A cosmetically acceptable composition or compound may be pharmaceutically acceptable. However, a cosmetically acceptable composition or compound need not be pharmaceutically acceptable.

As used herein, the term “pharmaceutically acceptable form” is meant to include known forms of a compound of the disclosure that may be administered to a subject, including, but not limited to, solvates, hydrates, prodrugs, isomorphs, polymorphs, pseudomorphs, neutral forms and salt forms of a compound of the disclosure.

As used herein, the term “pharmaceutically acceptable salt” refers to physiologically and pharmaceutically acceptable salts of the compounds of the invention: i.e., salts that retain the desired biological activity of the parent compound and do not impart undesired toxicological effects to the compounds disclosed. For oligonucleotides, exemplary pharmaceutically acceptable salts include but are not limited to (a) salts formed with cations such as sodium, potassium, ammonium, magnesium, calcium, polyamines such as spermine and spermidine and the like; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

As used herein, the term “pharmaceutical composition” refers to a mixture of one or more of the compounds of the disclosure, with other components, such as, but not limited to, pharmaceutically acceptable carriers and/or excipients. The purpose of a pharmaceutical composition is to facilitate administration of a compound of disclosure.

In some embodiments, a “cosmetically acceptable” excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

As used herein, the terms “repleted,” “repletion,” or “repleter” with respect to mtDNA refers to an increase in mtDNA copy number and/or concentration in a human or a non-human animal, tissue, or cell. Such a determination may be made with regard to a human or to a control non-human animal tissue, or cell that has undergone mtDNA depletion (such as immediately before repletion is initiated. In certain aspects, mtDNA is repletion results in mtDNA copy number and/or concentration approximately equal to the mtDNA copy number and/or concentration observed in a control non-human animal, tissue, or cell (for example, a non-human animal that has not expressed or does not express a mutant POLG1 polypeptide).

As used herein, the term “solvate” means a compound of the disclosure, or a pharmaceutically acceptable salt thereof, wherein one or more molecules of a suitable solvent are incorporated in the crystal lattice. A suitable solvent is physiologically tolerable at the dosage administered. Examples of suitable solvents are ethanol, water and the like. When water is the solvent, the molecule may be referred to as a “hydrate.”

As used herein, the terms “subject” or “patient” include all members of the animal kingdom including, but not limited to, vertebrates, mammals, animals (e.g., cats, dogs, horses, swine, rodents, etc.) and humans. In certain embodiments, the subject is a human. In certain embodiments, the subject is an animal, e.g., a research animal, e.g., a mouse, a rat.

As used herein, the terms “similarity” or “similarity score” when used with respect to a compound refers to a degree of similarity between two or more compounds in chemical structure, chemical function, and/or one or more chemical properties. The similarity score may be quantified by augmenting data generated by a deep neural network trained to model a plurality of chemical reactions for any given compound. The data may be augmented by a multi-dimensional vector defining a matrix of properties of the compound or a chemical reaction involving the compound to generate an embedding score for the compound. The embedding score for two or more compounds may be compared to generate the similarity score between the two or more compounds.

All patent applications, patents, and printed publications cited herein are incorporated herein by reference in their entireties, for all purposes.

### Impact of Mitochondrial Function on Intrinsic and Extrinsic Aging

Mitochondrial dysfunction is implicated in both intrinsic and extrinsic aging. The presence of skin wrinkles, acanthosis, epidermal hyperplasia with hyperkeratosis, and marked inflammatory infiltrate in the skin has been observed in mtDNA-depleter mice and represent characteristics similar to the extrinsic aging of skin in humans. Furthermore, the changes in expression of intrinsic aging-associated genetic markers support intrinsic mechanisms underlying the phenotypic changes observed in mtDNA-depleter mice.

Loss of collagen fibers is reported to underlie skin wrinkles. A tight balance between the proteolytic matrix metalloprotease (MMP) enzymes and their tissue-specific inhibitor tissue inhibitor metalloproteinase-1 (TIMP1) is essential to maintain the collagen fiber content in the skin. Expression of MMPs is altered in the aged skin. Consistent with these reports, the skin of mtDNA-depleter mice showed increased expression of MMPs and decreased expression of TIMP1, indicating loss of balance contributing to the development of skin wrinkles. Repletion of mtDNA content restored MMP expression leading to a reversal of wrinkled skin and hair loss. These experiments show that mitochondria are regulators of aging. This observation is surprising and suggests that epigenetic mechanisms underlying mitochondria-to-nucleus cross-talk must play an important role in the restoration of normal skin and hair phenotype.

mtDNA stress triggers inflammatory response. Inflammation also underlies aging and age-related diseases. Increased levels of markers of inflammation in the mtDNA-depleter mice indicate an activated immune response in the skin of mtDNA-depleter mice. Increased expression of NF-κB, a master regulator of the inflammatory response upon mtDNA depletion and its reduced expression after the restoration of mtDNA content suggests that NF-κB signaling is a critical mechanism contributing to the skin and hair follicle pathologies observed in mtDNA-depleter mice. Furthermore, a unique feature of proteins encoded by mtDNA is N-formyl-methionine at the N terminus. N-formylated peptides when present in the extracellular space are known to act as mitochondrial damage-associated molecular patterns and activate neutrophils or activate keratinocyte-intrinsic responses resulting in the recruitment of immune cells. While previous animal models have shown alternations in mtDNA homeostasis and/or mtDNA copy number or concentration using a localized approach (for example targeting only a specific cell type), the disclosure utilizes an animal model that provides for the global and targeted disruption of mtDNA homeostasis and/or mtDNA copy number of concentration in a controlled manner. Using such an animal model, the disclosure identified compounds that are effective in treating diseases and conditions relating to mitochondrial dysfunction in a background of significant mtDNA depletion. In addition, the disclosure demonstrates that such compounds reverse the physiological and phenotypic effects mediated by mitochondrial dysfunction. Exemplary physiological and phenotypic effects of intrinsic and extrinsic aging reversed include a decrease in skin wrinkles, a decrease in hair loss, an increase in hair follicles in growth phase, decreased inflammatory gene expression, decreased inflammatory infiltrate in the skin and hair follicles, and increased collagen content in the skin.

Further, due to the short lifespan of the animal models of the conventional practice, prior studies were prevented from determining the effect of mitochondrial dysfunction on the aging process and such studies did not observe the appearance of many of the physiological and phenotypic changes related to mitochondrial dysfunction. As such, the conventional practice was not capable of identifying compounds effective to treat such physiological and phenotypic changes related to mitochondrial dysfunction reported herein.

The foregoing limitations make identifying effective treatments for the skin diseases and conditions where mitochondrial dysfunction is at issue difficult. In addition, due to aberrations in normal epithelial development and hair follicle morphogenesis in current models, the issue of false positive and false negative results is high. The disclosure utilized an inducible non-human animal model expressing a mutated POLG1 polypeptide (such as, but not limited to, a POLG1 polypeptide expressing a dominant-negative (DN) mutation) that induces mitochondrial dysfunction (for example by depletion of mtDNA) in the whole animal or selected cells/tissues.

The non-human animal model allows for the ubiquitous suppression and restoration of mitochondrial function in the whole animal or in specific cells/tissues. The non-human animal model disclosed can be used to rapidly identify compounds effective in treating mtDNA-related diseases and conditions. The animal model in the absence of the expression of mutated POLG1 expression maintained normal epidermal differentiation and hair follicle morphogenesis making this animal model system a valuable tool in identifying therapies for the treatment of a variety of diseases and conditions characterized by mitochondrial dysfunction. When the mutant POLG1 polypeptide is expressed, the animal model demonstrates profound phenotypic age-related changes in the skin, including development of skin wrinkles and hair loss. The phenotypic changes are reversible when mitochondrial function is restored (such as through the administration of an extract or compound described herein).

### Non-Human Animal Model

In certain aspects, the methods described herein utilize a genetically modified non-human animal that expresses a mutant POLG1 polypeptide in a controlled manner. Tissues, organs and cells from such animal model may also be used. In one embodiment, the mutant POLG1 polypeptide is expressed ubiquitously (in every cell of the non-human animal). In another embodiment, the mutant POLG1 polypeptide is expressed in a specific tissue or set of tissues or in a specific cell type. Such non-human animal model is described in US Patent Publication No. 2020-0085021-A1, incorporated herein by reference in its entirety for all purposes.

In a particular aspect, the animal model exhibits at least one characteristic selected from the group consisting of: reduced mitochondrial (mt) DNA content, reduced mtDNA copy number, changes in mitochondrial protein expression, reduced expression of mitochondrial oxidative phosphorylation complexes, reduced stability of mitochondrial oxidative phosphorylation complexes, skin wrinkles, hair loss, increased epidermal thickness, epidermal hyperplasia, acanthosis, hyperkeratosis, altered, e.g., increased expression of at least one gene selected from the group consisting of: NF-κB, COX-2, INF-β1, CCL5, MMP1, MMP2, MMP9, MMP13, IGF1R, VEGF, and MRPS5, altered, e.g., decreased expression of at least one gene selected from the group consisting of: TIMP1, KLOTHO, COL1A1, MTCO2, TFAM, and VDAC, increased skin inflammation, and aberrant hair follicles.

### Methods of Screening for Therapeutic Agents

The disclosure provides an artificial neural network trained to model a plurality of chemical reactions for any given compound. Through the use of the artificial neural network described herein, compounds and compositions having a similarity score with a known promoter or inhibitor of mitochondrial biogenesis may be reliably identified. The similarity score may be quantified by augmenting data generated by the deep neural network in a multi-dimensional vector defining a matrix of properties of the compound or a chemical reaction involving the compound to generate an embedding score for the compound. The embedding score for the compound may be compared to an embedding score for the known promotor or inhibitor of mitochondrial biogenesis to generate the similarity score. Therefore, the disclosure provides for methods of screening compounds and compositions having a sufficient similarity score with known compounds. The identified compounds are predicted to be effective at promoting or inhibiting mitochondrial biogenesis.

The disclosure provides a non-human animal model comprising a mutant POLG1 polypeptide and that express the mutant POLG1 polypeptide in a controlled manner either throughout the animal or in specific tissues. Through the use of the non-human animal model described herein, compounds and compositions effective in treating diseases and conditions related to mitochondrial dysfunction can be reliably identified. Methods utilizing cells, and tissues from such a non-human animal are also provided. Therefore, the disclosure provides for methods of screening compounds and compositions effective to treat diseases and conditions related to mitochondrial dysfunction in a subject, including diseases and conditions related to mtDNA depletion.

In one embodiment, the disclosure provides for identification of a compound for the treatment of a disease or condition due, at least in part, to mitochodrial dysfunction, including changes in mtDNA copy number and/or concentration, and/or dysfunctional mitochondrial OXPHOS. Such diseases and conditions include, but are not limited to, mtDNA depletion syndromes, mitochondrial diseases, viral infections, viral infection-induced symptoms, aging, aging-associated chronic diseases, reduced energy levels and vitality, and other human pathologies. Exemplary mitochondrial diseases include cardiovascular disease, diabetes, cancer, neurological disorders, such as age-associated neurological disorders, skin diseases and conditions, e.g., skin wrinkles, changes in skin pigmentation, senile lentigines, characteristics of skin aging, hair or scalp diseases and conditions, e.g., hair loss, hair thinning, changes in hair pigmentation, e.g., hair graying.

In one embodiment, such a method of screening comprises the steps of: a) providing a deep neural network trained to model a plurality of chemical reactions for a known promoter or inhibitor of mitochondrial biogenesis; b) executing the deep neural network to identify one or more compounds having a threshold similarity score of at least 70% with the known promoter or inhibitor of mitochondrial biogenesis; c) selecting one or more identified compounds on the basis of at least one inclusion criteria; and d) evaluating the at least one selected compound in an assay to determine an effect on mitochondrial biogenesis by the selected compound, wherein the assay may include an in vitro assay, ex vivo assay, or in vivo assay. The at least one inclusion criteria may include a structural, functional, physical, or chemical property of the identified compound. Exemplary properties include size, charge, ionic strength, bond strength, valence, hybridization, micromolecular structure, macromolecular structure, and others. The threshold similarity score may be at least 70%, at least 75%, at least 80%, at least 85%, at least 90%, at least 95%, at least 99%, at least 99.5%, at least 99.9%, at least 99.95%, at least 99.99%, at least 99.995%, at least 99.999%, or greater.

In some embodiments, the assay may include screening the at least one selected compound with a non-human animal capable of inducible expression of a mutant POLG1 polypeptide, as described herein. In some embodiments, the assay may include screening the at least one selected compound to measure an effect on protein or gene expression of a mitochondrial health biomarker, as identified herein. The method of screening may be repeated by executing the deep neural network to identify one or more compounds having a similarity score of at least 70% with at least one identified compound and/or at least one selected compound. In some embodiments, the deep neural network may be retrained with at least one identified compound and/or at least one selected compound.

In one embodiment, such a method of screening comprises the steps of: a) providing a non-human animal capable of inducible expression of a mutant POLG1 polypeptide; b) stimulating the expression of the mutant POLG1 polypeptide, wherein stimulating expression of the mutant POLG1 polypeptide induces a physiological or phenotypic response; c) administering an agent to the non-human animal either before step b) or after step b); d) determining the effect of the agent on pathology; and e) comparing the effect of the agent to a control animal, wherein a reduction or an increase (as appropriate) in the physiological or phenotypic response in the non-human animal after administration of the agent indicates the agent is a therapeutic agent for the treatment of the physiological or phenotypic response.

In another embodiment, the disclosure provides a method for identifying a therapeutic agent for the treatment of mitochondrial dysfunction.

In another embodiment, the disclosure provides a method for identifying a therapeutic agent for the treatment of a disease or condition associated with mitochondrial dysfunction or a symptom thereof.

In another embodiment, the disclosure provides a method for identifying a therapeutic agent for the treatment of an aging-associated chronic disease or condition related to mitochondrial dysfunction or a symptom thereof.

In another embodiment, the disclosure provides a method for identifying a therapeutic agent for improving or preventing a decrease in energy level and/or vitality.

In another embodiment, the disclosure provides a method for identifying a therapeutic agent for treating or preventing viral infection-induced symptoms and/or treating or preventing a viral infection.

In any of the described methods of screening, agents can include, but are not limited to, chemical compounds, pharmaceutical compositions, cosmetic composition, extracts, plant extracts, seaweed extracts, microbial extracts, biological compounds and compositions (e.g., proteins, DNA, RNA, siRNAs, vaccines and the like), and microorganisms. Further, the agent may be selected from a library, including a library of agents approved by a regulatory authority such as the FDA.

In any of the described methods of screening, any of the transgenic non-human animals of the disclosure may be used.

In any of the described methods of screening, step b) may be accomplished by providing an inducer compound to the transgenic non-human animal or withholding the inducer compound from the transgenic non-human animal.

In any of the described methods of screening, the agent is added before step b). In any of the described methods of screening, the agent is added after step b).

In any of the described methods of screening, the animal model is an animal model described in the preceding section. In any of the described methods of screening, the mutant POLG1 polypeptide may be any mutant POLG1 polypeptide described herein. In certain aspects, the mutant POLG1 polypeptide comprises a dominant negative mutation. In certain aspects, the mutant POLG1 polypeptide comprises a D1135A mutation.

### Methods of Treatment

The disclosure provides compounds and compositions effective to treat or prevent diseases and conditions related to mitochondrial dysfunction, including mtDNA depletion. Diseases and conditions related to mitochondrial dysfunction include viral infections and symptoms thereof. In certain embodiments, the viral infections and/or viral infection-induced symptoms include those associated with mitochondrial dysfunction. While not being bound to any particular theory, such compounds may exert the observed effects through inhibiting a decrease in mitochondrial DNA copy number or concentration, inhibiting depletion of mitochondrial DNA, inhibiting degradation of mitochondrial DNA, contributing to an increase in mitochondrial DNA copy number or concentration, repleting mitochondrial DNA, and/or promoting increased activity of mitochondrial DNA. Additionally, it is believed the compounds disclosed herein may provide anti-viral treatment by decreasing or inhibiting an increase in circulating mitochondrial DNA levels, for example, plasma DNA and/or cytoplasmic DNA.

In one embodiment, the disclosure provides a method for treating or preventing mitochondrial dysfunction in a subject, the method comprising administering to said subject an effective amount of a compound of the disclosure, or a pharmaceutically acceptable form thereof. In one embodiment, the disclosure provides a method for treating or preventing a disease or condition associated with mitochondrial dysfunction or a symptom thereof in a subject, the method comprising administering to said subject an effective amount of a compound of the disclosure, or a pharmaceutically acceptable form thereof.

In one embodiment, the disclosure provides a method for treating or preventing viral infection-induced symptoms, the method comprising administering to said subject an effective amount of a compound of the disclosure, or a pharmaceutically acceptable form thereof.

The viral infection-induced symptoms may include one or more acute and/or chronic symptoms. The viral infection-induced symptoms may include respiratory symptoms, gastrointestinal symptoms, cardiac symptoms, neurological symptoms, and/or loss of energy or vitality. For example, the viral infection-induced symptoms may include muscle or body aches, fatigue, shortness of breath, difficulty breathing, fever or chills, headache, loss of taste or smell, sore throat, congestion or runny nose, nausea or vomiting, diarrhea, cough, lymphadenitis, rash, or sleep hyperhidrosis. Other viral infection-induced symptoms are within the scope of the disclosure.

In one embodiment, the disclosure provides a method for treating or preventing a viral infection, the method comprising administering to said subject an effective amount of a compound of the disclosure, or a pharmaceutically acceptable form thereof.

The viral infection may be any viral infection related to mitochondrial dysfunction. For example, the viral infection and/or symptom thereof may be associated with human coronavirus, e.g., HCoV-229E, severe acute respiratory syndrome (SARS), e.g., SARS-CoV-2 or SARS-CoV-1, human immunodeficiency virus (HIV), influenza, e.g., influenza A or B, or Middle East respiratory syndrome (MERS). Other viral infections are within the scope of the disclosure.

Certain extracts have been identified as comprising one or more compounds that promote and/or inhibit mtDNA. Emblica extract, fucus extract, and chebula extract are described herein. It should be understood that similar extracts, in particular, extracts comprising one or more compounds disclosed herein, compounds that are constituents of an extract disclosed herein, and/or compounds having a similarity score of at least 95% with one or more compounds disclosed herein, are expected to provide similar mtDNA promotion and/or inhibition. Accordingly, other extracts, compounds derived from, constituents of, or purified from other extracts, and compounds having a similarity score of at least 95% with compounds derived from, constituents of, or purified from other extracts are within the scope of the disclosure.

Exemplary extracts include Polygonum aviculare extract, Physalis gngulata extract, Dunaliella salina extract, Camellia sinensis leaf extract, Tremella fuciformis sporocarp extract, Alteromonas ferment extract, Theobroma cacao (cocoa) seed extract, Vitis vinifera (grape) flower cell extract, Mirabilis jalapa callus extract, Alteromonas ferment extract, and Vibrio alginolyticus ferment filtrate.

Accordingly, the compounds described herein may be derived from, purified from, or isolated from the extract. The compounds described herein may be derived from, purified from, or isolated from a source other than the extract. A compound constituent of an extract may be derived from, purified from, or isolated from another natural or artificial source. In other embodiments, the compounds described herein may be synthetic. For instance, the compounds of the disclosure may be synthesized in a laboratory, manufacturing, or other setting. The above applies to compounds contained in or constituents of an extract, as well as compounds having a high similarity score thereof.

The methods disclosed herein may comprise administering to said subject an effective amount of an emblica extract, or a pharmaceutically acceptable form thereof.

The methods disclosed herein may comprise administering to said subject an effective amount of a compound derived from, constituent of, or purified from an emblica extract, or a pharmaceutically acceptable form thereof, or a compound having a similarity score of at least 95% with the compound derived from, constituent of, or purified from an emblica extract, or a pharmaceutically acceptable form thereof.

The methods disclosed herein may comprise administering to said subject an effective amount of a fucus extract, or a pharmaceutically acceptable form thereof.

The methods disclosed herein may comprise administering to said subject an effective amount of a compound derived from, constituent of, or purified from a fucus extract, or a pharmaceutically acceptable form thereof, or a compound having a similarity score of at least 95% with the compound derived from, constituent of, or purified from a fucus extract, or a pharmaceutically acceptable form thereof.

The methods disclosed herein may comprise administering to said subject an effective amount of a chebula extract, or a pharmaceutically acceptable form thereof.

The methods disclosed herein may comprise administering to said subject an effective amount of a compound derived from, constituent of, or purified from a chebula extract, or a pharmaceutically acceptable form thereof, or a compound having a similarity score of at least 95% with the compound derived from, constituent of, or purified from a chebula extract, or a pharmaceutically acceptable form thereof.

The disclosure may generally be related to extracts and compounds derived from, constituents of, or purified from extracts. It should be understood that compounds having a similarity score of at least 95%, for example, at least 96%, at least 97%, at least 98%, at least 99%, at least 99.5%, at least 99.9%, at least 99.95%, at least 99.99%, at least 99.995%, or at least 99.999% with a compound derived from, constituents of, or purified from the extract may be utilized in any composition as described herein.

Without wishing to be bound by theory, it is believed the administration of the compositions disclosed herein may involve inhibition of a decrease in mitochondrial DNA copy number or concentration, inhibition of a depletion of mitochondrial DNA, inhibition of a degradation of mitochondrial DNA, or a combination of the foregoing. Without wishing to be bound by theory, it is believed the administration of the compositions disclosed herein may involve inducing mitochondrial biogenesis and/or improving mitochondrial function.

In accordance with certain aspects, administration of the compositions disclosed herein may involve an increase in expression of mitochondrial oxidative phosphorylation complexes, an increase in stability of mitochondrial oxidative phosphorylation complexes, and/or an increase in expression of at least one protein selected from PGC-1a, TFAM, NRF-1, and COXII.

In certain aspects, the emblica extract is derived from Emblica officinalis (also known as Phyllanthus emblica, Indian gooseberry, or by its Hindi name Amla). Such an extract from Emblica officinalis may be derived from any portion of the plant as desired. For example, the extract may be derived from the stem portion, the fruit portion, or both the stem portion and the fruit portion of Emblica officinalis. In preparing such an extract, the Emblica officinalis may be provided in a powdered from and extracted using chemical solvents known in the art, such as, but not limited to, aqueous ethyl acetate ethanol, and methanol. Other solvents or excipients disclosed herein may be used. In certain exemplary embodiments, the chemical solvent may be methanol.

In certain aspects, the effective amount of an emblica extract or a compound derived from, constituent of, or purified from an emblica extract is from 1 to 100 mg, such as 1 mg, 5, mg, 10 mg, 20 mg, 30 mg, 40 mg, 50 mg, 60 mg, 70 mg, 80 mg, 90 mg, or 100 mg of

In certain aspects, the compound derived from, constituent of, or purified from an emblica extract is a breakdown product of a tannin, wherein the emblica extract is optionally as described above. In certain aspects, the compound derived from, constituent of, or purified from an emblica extract is an ellagitannin. In certain aspects, the compound derived from, constituent of, or purified from an emblica extract is emblicanin A, emblicanin B, punigluconin, pedunculagin and/or chebulinic acid.

In certain aspects, the compound is an ellagitannin or a compound having a similarity score of at least 95% with an ellagitannin. Exemplary ellagitannins and compounds having a high similarity score with such ellagitannins are listed in Table 1. Other compounds having a high similarity score with ellagitannins are within the scope of the disclosure.

TABLE 1
Ellagitannins and compounds having a high similarity score with ellagitannins

| Ellagitannin | Compound having high similarity score with ellagitannin |
|---|---|
| Emblicanin b | Eugeniin |
| Emblicanin a | 1,2,3,4-Tetrakis-O-Galloyl-Alpha-D-Glucose |
| Emblicanin a | 1,2,3,6-Tetragalloylglucose |
| Emblicanin b | Punicafolin |
| Emblicanin a | 4-hydroxy-3,5-bis(3,4,5-trihydroxybenzoyloxy)- |
| | 6-[(3,4,5-trihydroxybenzoyloxy)methyl]oxan- |
| | 2-yl 3,4,5-trihydroxybenzoate |
| Emblicanin b | Punicalin |
| Emblicanin a | Thonningianin A |
| Emblicanin a | Sagerinic Acid |
| Emblicanin b | Casuariin |
| Emblicanin a | Prunasin 2′,3′,4′,6′-Tetra-O-Gallate |
| Emblicanin b | Epigallocatechin-(4Beta->8)- |
| | Epigallocatechin-3-O-Gallate |
| Emblicanin b | Arjuna |
| Emblicanin a | Hyemaloside A |
| Emblicanin b | Trapain |
| Emblicanin a | 4-[3,4-dihydroxy-5-(3,4,5- |
| | trihydroxybenzoyloxy)benzoyloxy]-1-hydroxy- |
| | 3,5-bis(3,4,5-trihydroxybenzoyloxy)cyclohexane-1- |
| | carboxylic acid |
| Emblicanin a | Pentagalloylglucose |
| Emblicanin a | Myricetin 3-(2″,3″-Digalloylrhamnoside) |
| Emblicanin b | Trilobatin G |
| Emblicanin b | Stachyurin |
| Emblicanin a | Eutannin |
| Emblicanin b | Thonningianin A |
| Emblicanin b | Castalagin |
| Emblicanin b | Casuarictin |
| Emblicanin a | Yunnaneic Acid G |
| Emblicanin a | Rabdosiin |
| Emblicanin b | 2-(3,4-dihydroxyphenyl)-5,7-dihydroxy-8-[3,5,7- |
| | trihydroxy-2-(3,4,5-trihydroxyphenyl)-3,4- |
| | dihydro-2H-1-benzopyran-4-yl]-3,4- |
| | dihydro-2H-1-benzopyran-3-yl 3,4,5-trihydroxybenzoate |
| Emblicanin b | Pterocaryanin c |
| Emblicanin b | Tercatain |
| Emblicanin a | Trapain |
| Emblicanin b | Tellimagrandin I |
| Emblicanin b | Sagerinic Acid |
| Emblicanin b | Paeonianin E |
| Emblicanin a | 2-(3-{[1-carboxy-2-(3,4- |
| | dihydroxyphenyl)ethoxy]carbonyl}-2-(3,4- |
| | dihydroxyphenyl)-7,8-dihydroxy-1,2- |
| | dihydronaphthalene-1-carbonyloxy)- |
| | 3-(3,4-dihydroxyphenyl)propanoic acid |
| Emblicanin a | 2-{[2-(3,4-dihydroxyphenyl)-5,7-dihydroxy- |
| | 4-oxo-4H-chromen-3-yl]oxy}-4,5-dihydroxy- |
| | 6-[(3,4,5-trihydroxybenzoyloxy)methyl] |
| | oxan-3-yl 3,4,5-trihydroxybenzoate |
| Emblicanin a | Remurin B |
| Emblicanin a | Arjuna |
| Emblicanin a | 5-Hydroxyferuloyl-Coa |
| Emblicanin a | Geraniinic Acid B |
| Emblicanin a | Casuariin |
| Emblicanin a | Eugeniin |
| Emblicanin a | Sinapoyl-Coa |
| Emblicanin b | Geraniin |
| Emblicanin a | Feruloyl-Coa |
| Emblicanin a | Balanophotannin A |
| Emblicanin b | 6-(4-{7-[(6-carboxy-3,4,5-trihydroxyoxan-2- |
| | yl)oxy]-5-hydroxy-4-oxo-4H-chromen-2- |
| | yl}phenoxy)-5-[(6-carboxy-4,5-dihydroxy-3- |
| | {[3-(4-hydroxyphenyl)prop-2- |
| | enoyl]oxy}oxan-2-yl)oxy]-3,4- |
| | dihydroxyoxane-2-carboxylic acid |
| Emblicanin a | Chebulagic Acid |
| Emblicanin b | Eutannin |
| Emblicanin b | Balanophotannin A |
| Emblicanin b | Myricetin 3-(2″,3″-Digalloylrhamnoside) |
| Emblicanin b | Pradimicin C |
| Emblicanin b | 5,5″-Bis-[2,3-Dicarboxy-6,7-Dihydroxy-1-(3′,4′- |
| | Dihy droxyphenyl)- |
| | 1,2-Dihydronaphthalene] |
| Emblicanin a | Stachyurin |
| Emblicanin a | Punicafolin |
| Emblicanin b | Alnusiin |
| Emblicanin b | Phyllanthusiin D |
| Emblicanin a | Caffeoyl-Coa |

In certain aspects, the compound is chebulinic acid or a compound having a similarity score of at least 95% with chebulinic acid (formula I below). Exemplary compounds having a high similarity score with chebulinic acid are listed in Table 2.

TABLE 2
Compounds having a high similarity score with chebulinic acid.

| Compound ID | |
|---|---|
| from COCONUT | |
| Database | Structure |
| CNP0285899 | 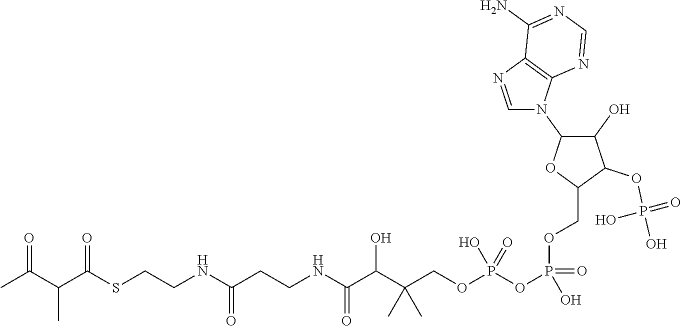 [SMILES: CC(=O)C(C)C(=O)SCCNC(=O)CCNC(=O)C(O)C(C)(C)COP(=O)(O)OP(=O)(O)OCC1OC(n2cnc3c(N)ncnc32)C(O)C1OP(=O)(O)O] |
| CNP0059074 | 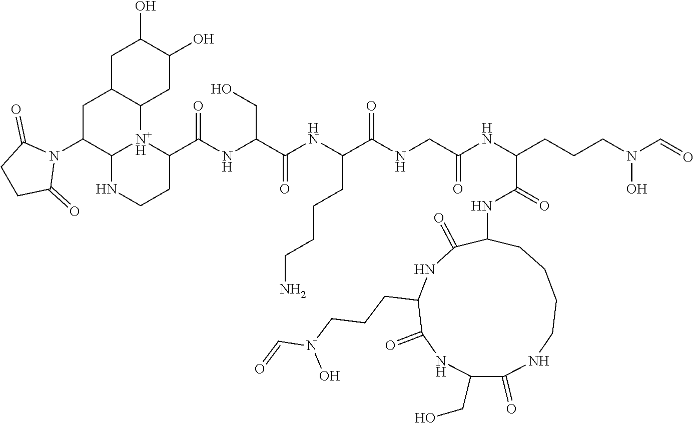 |
| CNP0343660 | 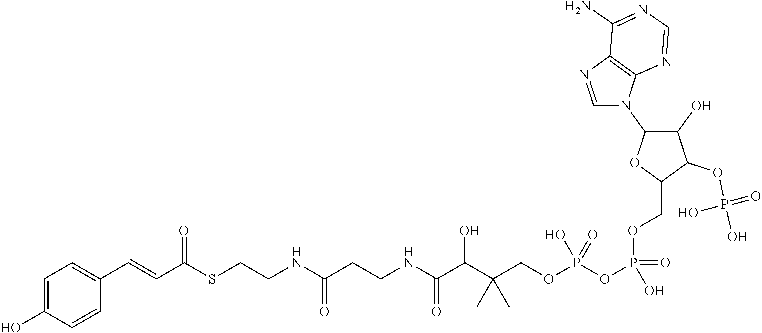 [SMILES: CC(C)(COP(=O)(O)OP(=O)(O)OCC1OC(n2cnc3c(N)ncnc32)C(O)C1OP(=O)(O)O)C(O)C(=O)NCCC(=O)NCCSC(=O)/C=C/c1ccc(O)cc1] |
| CNP0139083 | 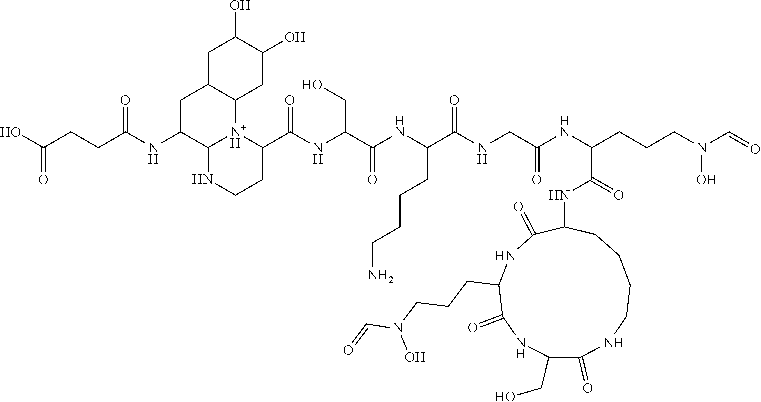 |
| CNP0041318 | 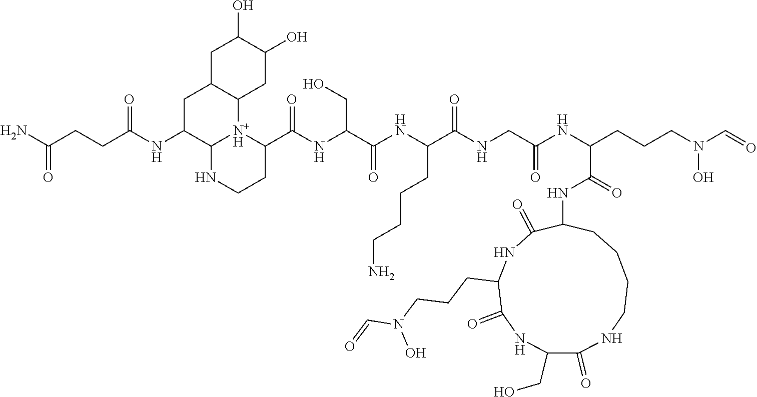 |
| CNP0422373 | 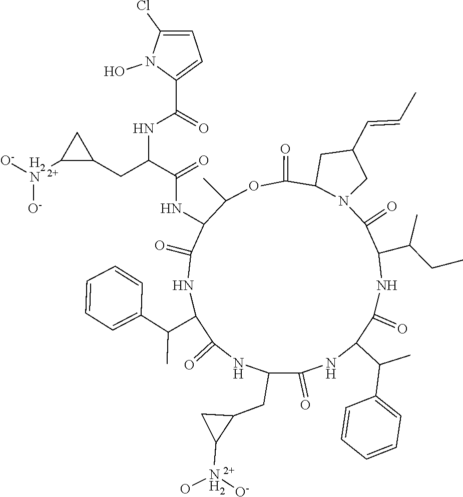 |
| CNP0415802 | 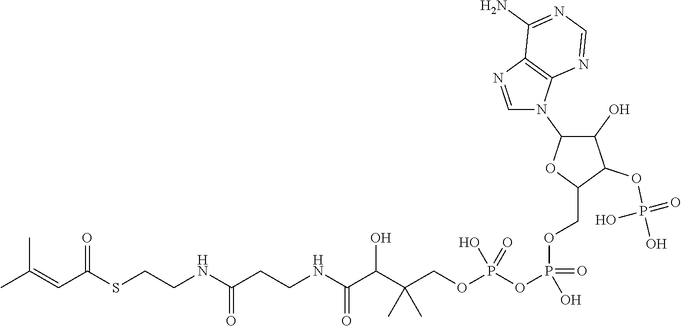 [SMILES: CC(C)=CC(=O)SCCNC(=O)CCNC(=O)C(O)C(C)(C)COP(=O)(O)OP(=O)(O)OCC1OC(n2cnc3c(N)ncnc32)C(O)C1OP(=O)(O)O] |
| CNP0070782 | 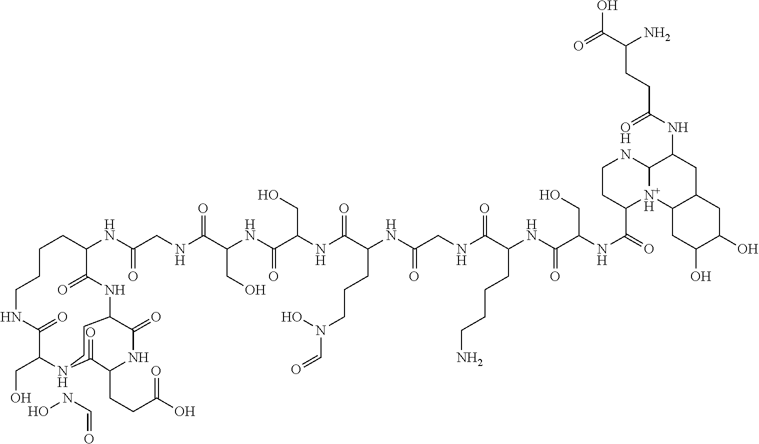 |
| CNP0102046 | 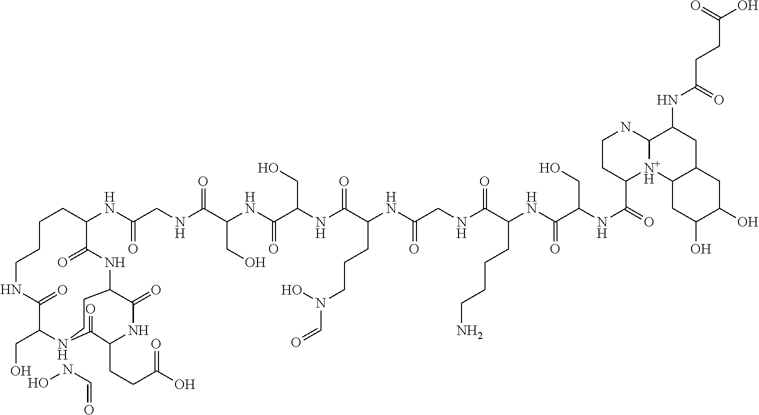 |
| CNP0098207 | 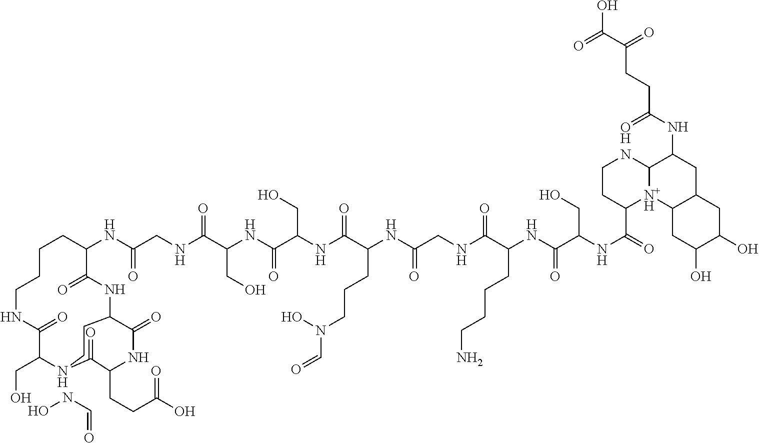 |
| CNP0026197 | 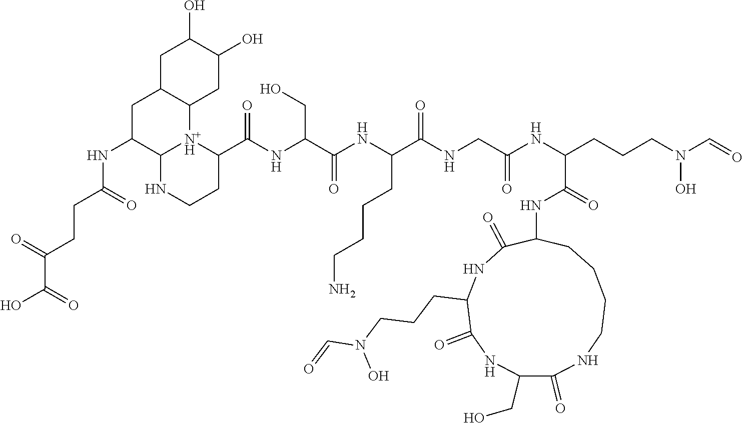 |
| CNP0326396 | 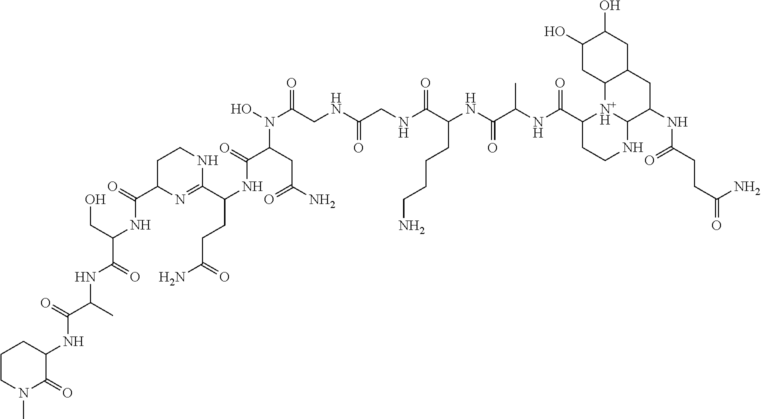 |
| CNP0384812 | 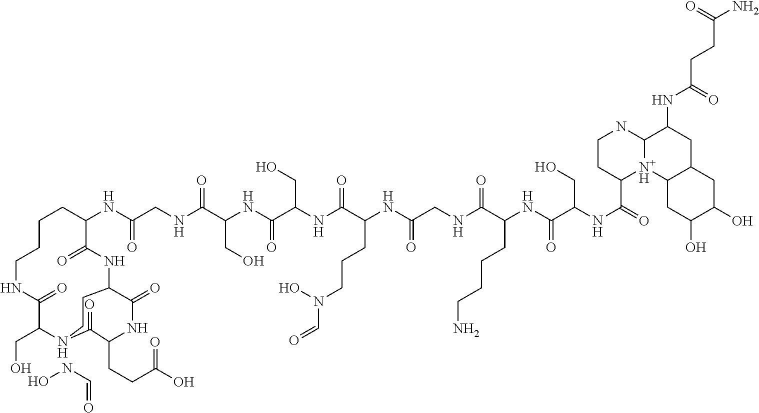 |
| CNP0024232 | 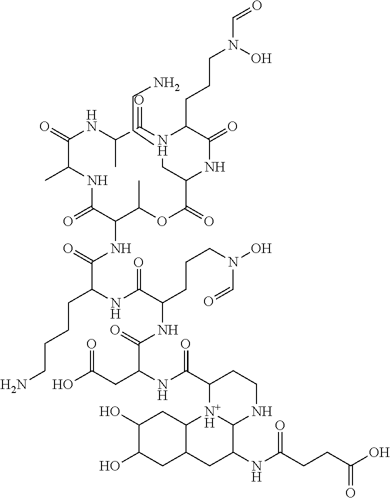 |
| CNP0070582 | 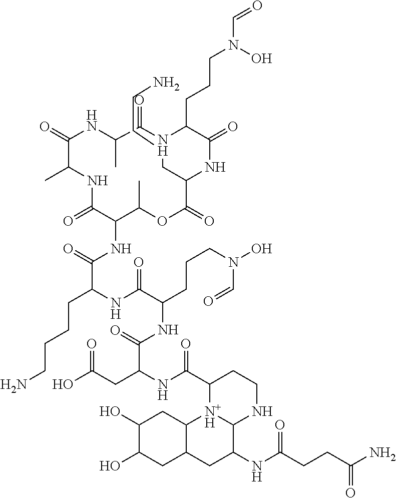 |

In certain aspects, the compound derived from, constituent of, or purified from an emblica extract is a benzoic acid substituted with 1 to 5 hydroxy groups and optionally 1 to 3 O—(C₁-C₅ alkyl) or O—(C₁-C₅ alkenyl) groups. In certain aspects, the compound derived from, constituent of, or purified from an emblica extract is a benzoic acid substituted with 1 to 3 hydroxy groups and optionally 1 to 2 O—(C₁-C₅ alkyl) or O—(C₁-C₅ alkenyl) groups.

In certain aspects, the compound derived from, constituent of, or purified from an emblica extract is a benzene substituted with —CH═CH—(CH₂)ₐ—C(O)OH, wherein a is 0 to 5, and 1 to 5 hydroxy groups. In certain aspects, the compound derived from, constituent of, or purified from an emblica extract is a benzene substituted with —CH═CH—(CH₂)ₐ—C(O)OH, wherein a is 0 to 5, and 1 to 3 hydroxy groups. In certain aspects, the compound derived from, constituent of, or purified from an emblica extract is a benzene substituted with —CH═CH—(CH₂)ₐ—C(O)OH, wherein a is 0, and 1 to 3 hydroxy groups.

In certain aspects, the compound derived from, contained in, or purified from an emblica extract is gallic acid, vanillic acid, chlorogenic acid, caffeic acid, syringic acid, coumaric aid, quercetin, emblicanin A, emblicanin B, punigluconin, and pedunculagin, punicafolin, phyllanemblin, kaempferol, ellagic acid, chebulinic acid, chebulagic acid, punicalagin, or a metabolite of any of the foregoing.

In certain aspects, the compound derived from, constituent of, or purified from an emblica extract is ascorbic acid or citric acid.

In certain aspects, the compound derived from, constituent of, or purified from an emblica extract is gallic acid, vanillic acid, chlorogenic acid, caffeic acid, syringic acid, coumaric aid, quercetin, vitamin C, or a metabolite of any of the foregoing.

In certain aspects, the compound derived from, constituent of, or purified from an emblica extract is gallic acid or a compound having a similarity score of at least 95% with gallic acid.

In certain aspects, the active agent in the composition is gallic acid. The active agent may be or comprise an ellagitannin. The active agent may be or comprise emblicanin A, emblicanin B, punigluconin, pedunculagin, and/or chebulinic acid.

In accordance with certain embodiments, the composition may comprise an emblica extract fortified with one or more compound that is a constituent of an emblica extract. The compound constituent of the emblica extract or combination of compounds constituents of the emblica extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from an emblica extract, optionally the effective amount of an active agent of the composition, is from 1 to 100 mg of the, such as 1 mg, 5 mg, 10 mg, 20 mg, 30 mg, 40 mg, 50 mg, 60 mg, 70 mg, 80 mg, 90 mg, or 100 mg of the compound derived from, constituent of, or purified from an emblica extract.

In certain aspects, the fucus extract is derived from Fucus vesiculosus, Fucus serratus, Fucus, spiralis, or Fucus guiryi. In certain aspects, the fucus extract is derived from Fucus vesiculosus. Such an extract may be derived from any portion of the algae as desired. In preparing such an extract, the Fucus vesiculosus, Fucus serratus, Fucus, spiralis, or Fucus guiryi may be provided in a powdered from and extracted using chemical solvents known in the art, such as, but not limited to, aqueous ethyl acetate ethanol, and methanol. Other solvents or excipients disclosed herein may be used. In certain exemplary embodiments, the chemical solvent may be methanol.

In certain aspects, the effective amount of fucus extract is from 1 to 100 mg of extract, such as 1 mg, 5 mg, 10 mg, 20 mg, 30 mg, 40 mg, 50 mg, 60 mg, 70 mg, 80 mg, 90 mg, or 100 mg of a fucus extract.

In certain aspects, the compound derived from, constituent of, or purified from a fucus extract is a breakdown product of a tannin. In certain aspects, the compound derived from, constituent of, or purified from a fucus extract is an ellagitannin. In certain aspects, the compound derived from, constituent of, or purified from a fucus extract is fucoidan.

In certain aspects, the compound derived from, constituent of, or purified from a fucus extract is a benzoic acid substituted with 1 to 5 hydroxy groups and optionally 1 to 3 O—(C₁-C₅ alkyl) or O—(C₁-C₅ alkenyl) groups. In certain aspects, the compound derived from, constituent of, or purified from a fucus extract is a benzoic acid substituted with 1 to 3 hydroxy groups and optionally 1 to 2 O—(C₁-C₅ alkyl) or O—(C₁-C₅ alkenyl) groups.

In certain aspects, the compound derived from, constituent of, or purified from a fucus extract is a benzene substituted with —CH═CH—(CH₂)ₐ—C(O)OH, wherein a is 0 to 5, and 1 to 5 hydroxy groups. In certain aspects, the compound derived from, constituent of, or purified from a fucus extract a benzene substituted with —CH═CH—(CH₂)ₐ—C(O)OH, wherein a is 0 to 5, and 1 to 3 hydroxy groups. In certain aspects, the compound derived from, constituent of, or purified from a fucus extract a benzene substituted with —CH═CH—(CH₂)ₐ—C(O)OH, wherein a is 0, and 1 to 3 hydroxy groups.

In certain aspects, the compound derived from, constituent of, or purified from a fucus extract is gallic acid, vanillic acid, chlorogenic acid, caffeic acid, syringic acid, coumaric aid, or a metabolite of any of the foregoing.

In certain aspects, the compound derived from, constituent of, or purified from a fucus extract is gallic acid.

In certain aspects, the active agent in the composition is gallic acid or a compound having a similarity score of at least 95% with gallic acid. In certain aspects, the active agent in the composition is chebulinic acid or a compound having a similarity score of at least 95% with chebulinic acid (see, e.g., Table 2 above). The active agent may be or comprise an ellagitannin or a compound having a similarity score of at least 95% with an ellagitannin (see, e.g., Table 1 above). The active agent may be or comprise fucoidan or a compound having a similarity score of at least 95% with fucoidan.

In accordance with certain embodiments, the composition may comprise a fucus extract fortified with one or more compound that is a constituent of a fucus extract. The compound constituent of a fucus extract or combination of compounds constituents of a fucus extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from a fucus extract, optionally the effective amount of an active agent of the composition, is from 1 to 100 mg, such as 1 mg, 5 mg, 10 mg, 20 mg, 30 mg, 40 mg, 50 mg, 60 mg, 70 mg, 80 mg, 90 mg, or 100 mg of the compound derived from, constituent of, or purified from a fucus extract.

In certain aspects, the chebula extract is derived from Terminilia chebula, Terminalia arborea, or Lumnitzera racemose. In certain aspects, the chebula extract is derived from Termibnilia chebula. Such an extract may be derived from any portion of the plant as desired. In preparing such an extract, the Terminilia chebula, Terminalia arborea, or Lumnitzera racemose may be provided in a powdered from and extracted using chemical solvents known in the art, such as, but not limited to, aqueous ethyl acetate ethanol, and methanol. Other solvents or excipients disclosed herein nay be used. In certain exemplary embodiments, the chemical solvent may be methanol.

In certain aspects, the effective amount of chebula extract is from 1 to 100 mg of extract, such as 1 mg, 5 mg, 10 mg, 20 mg, 30 mg, 40 mg, 50 mg, 60 mg, 70 mg, 80 mg, 90 mg, or 100 mg of a chebula extract.

In certain aspects, the compound derived from, constituent of, or purified from a chebula extract is a breakdown product of a tannin. In certain aspects, the compound derived from, constituent of, or purified from a chebula extract is an ellagitannin. In certain aspects, the compound derived from, constituent of, or purified from a chebula extract is chebulinic acid.

In certain aspects, the compound derived from, constituent of, or purified from a chebula extract is a benzoic acid substituted with 1 to 5 hydroxy groups and optionally 1 to 3 O—(C₁-C₅ alkyl) or O—(C₁-C₅ alkenyl) groups. In certain aspects, the compound derived from, constituent of or purified from a chebula extract is a benzoic acid substituted with 1 to 3 hydroxy groups and optionally 1 to 2 O—(C₁-C₅ alkyl) or O—(C₁-C₅ alkenyl) groups.

In certain aspects, the compound derived from, constituent of, or purified from a chebula extract is a benzene substituted with —CH═CH—(CH₂)ₐ—C(O)OH, wherein a is 0 to 5, and 1 to 5 hydroxy groups. In certain aspects, the compound derived from, constituent of, or purified from a chebula extract a benzene substituted with —CH═CH—(CH₂)ₐ—C(O)OH, wherein a is 0 to 5, and 1 to 3 hydroxy groups. In certain aspects, the compound derived from, constituent of, or purified from a chebula extract a benzene substituted with —CH═CH—(CH₂)ₐ—C(O)OH, wherein a is 0, and 1 to 3 hydroxy groups.

In certain aspects, the compound derived from, constituent of, or purified from a chebula extract is gallic acid, vanillic acid, chlorogenic acid, caffeic acid, syringic acid, coumaric aid, or a metabolite of any of the foregoing.

In certain aspects, the compound derived from, constituent of, or purified from a fucus extract is chebulinic acid.

In certain aspects, the active agent in the composition is chebulinic acid or a compound having a similarity score of at least 95% with chebulinic acid (see, e.g., Table 2 above). The active agent may be or comprise an ellagitannin or a compound having a similarity score of at least 95% with an ellagitannin (see, e.g., Table 1 above).

In accordance with certain embodiments, the composition may comprise a chebula extract fortified with one or more compound that is a constituent of a chebula extract. The compound constituent of a chebula extract or combination of compounds constituents of a chebula extract may be purified, e.g., at least 80% purified, at least 85% purified, at least 90% purified, at least 95% purified, at least 98% purified, at least 99% purified, at least 99.9% purified, at least 99.99% purified, or at least 99.999% purified.

In certain aspects, the effective amount of a compound derived from, constituent of, or purified from a chebula extract, optionally the effective amount of an active agent of the composition, is from 1 to 100 mg, such as 1 mg, 5 mg, 10 mg, 20 mg, 30 mg, 40 mg, 50 mg, 60 mg, 70 mg, 80 mg, 90 mg, or 100 mg of the compound derived from, constituent of, or purified from a chebula extract.

The methods, and any of aspects of the foregoing, may comprise administering a composition comprising an effective amount of two or more of an emblica extract or a compound constituent of an emblica extract, an effective amount of a fucus extract or a compound constituent of a fucus extract, and an effective amount of a chebula extract or a compound constituent of a chebula extract. In accordance with certain embodiments, the two or more of the emblica extract or compound constituent of the emblica extract, the fucus extract or compound constituent of the fucus extract, and the chebula extract or compound constituent of the chebula extract may provide synergistic effects in the treatment of the disease or condition.

The compositions disclosed herein may comprise or be fortified with one or more of: an emblica extract or compound constituent of an emblica extract, a fucus extract or compound constituent of a fucus extract, a chebula extract or compound constituent of the chebula extract, BAMLET 10, BAMLET 50, ferulic acid, quercetin, urolithin A, pterostilbene, acadesine, embelin, EGCG, eriocitrin, gallic acid, gomsin A, lutein, luteolin, NAD, rutin, zeaxanthin, and melatonin.

The methods, and any aspects of the foregoing, may further comprise one or more of the steps: i) identifying a subject in need or treatment; and (ii) providing a compound of the disclosure or a pharmaceutical composition comprising a compound of the disclosure.

In any of the foregoing embodiments, and any aspects of the foregoing, when the term “preventing” is used the term may refers to at least a partial inhibition, for example a 10% inhibition, a 20% inhibition, a 30% inhibition, a 40% inhibition, 50% inhibition, a 60% inhibition, a 70% inhibition, an 80% inhibition, a 90% inhibition, a 95% inhibition or greater than 95% inhibition.

In any of the foregoing embodiments, and any aspects of the foregoing, the compound, or pharmaceutically acceptable form thereof, may be administered alone or as a part of a pharmaceutical composition. The pharmaceutical composition may be formulated by combining a solution of the compound with a pharmaceutically suitable carrier. The solution of the compound may comprise 1 to 1,000 mg of the compound, for example, 10 to 600 mg, 20 to 500 mg, 30 to 200 mg, 50 to 100 mg, or 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, or 1,000 mg of the compound.

The compound and the pharmaceutically suitable carrier may be combined in a ratio of 1:5 to 5:1. The pharmaceutical composition may be formulated to have a concentration of 1 to 1,000 mg/ml of the compound, for example, 10 to 600 mg/ml, 20 to 500 mg/ml, 30 to 200 mg/ml, 50 to 100 mg/ml, or 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 200 mg/ml, 300 mg/ml, 400 mg/ml, 500 mg/ml, 600 mg/ml, 700 mg/ml, 800 mg/ml, 900 mg/ml, or 1,000 mg/ml of the compound.

The pharmaceutical composition may be formulated to have a concentration of 0.01% to 2% of the compound, for example, 0.01%, 0.02%, 0.05%, 0.1%, 0.2%, 0.5%, 1%, or 2% of the compound.

The pharmaceutical composition may be formulated to have a concentration of 2.5 to 50 μM of the compound, for example, 2.5 μM, 5 μM, 10 μM, 25 μM, or 50 μM of the compound. The pharmaceutical composition may be formulated to have a concentration of 50 to 500 μM of the compound, for example, 50 μM, 100 μM, 200 μM, 300 μM, 400 μM, or 500 μM of the compound.

The pharmaceutical composition may be formulated to have a concentration of 2.5 to 50 μM of the compound, for example, 2.5 μg/L, 5 μg/L, 10 μg/L, 25 μg/L, or 50 μg/L of the compound. The pharmaceutical composition may be formulated to have a concentration of 50 to 500 μg/L of the compound, for example, 50 μg/L, 100 μg/L, 200 μg/L, 300 μg/L, 400 μg/L, or 500 μg/L of the compound.

In any of the foregoing embodiments, and any aspects of the foregoing, the compound, or pharmaceutically acceptable form thereof, may be administered alone or as a part of a cosmetic composition. The cosmetic composition may be formulated by combining a solution of the compound with a cosmetically suitable carrier. The solution of the compound may comprise 1 to 1,000 mg of the compound, for example, 10 to 600 mg, 20 to 500 mg, 30 to 200 mg, 50 to 100 mg, or 1 mg, 5 mg, 10 mg, 20 mg, 30 mg, 40 mg, 50 mg, 60 mg, 70 mg, 80 mg, 90 mg, 100 mg, 200 mg, 300 mg, 400 mg, 500 mg, 600 mg, 700 mg, 800 mg, 900 mg, or 1,000 mg of the compound.

The compound and the cosmetically suitable carrier may be combined in a ratio of 1:5 to 5:1. The cosmetic composition may be formulated to have a concentration of 1 to 1,000 mg/ml of the compound, for example, 10 to 600 mg/ml, 20 to 500 mg/ml, 30 to 200 mg/ml, 50 to 100 mg/ml, or 1 mg/ml, 5 mg/ml, 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 200 mg/ml, 300 mg/ml, 400 mg/ml, 500 mg/ml, 600 mg/ml, 700 mg/ml, 800 mg/ml, 900 mg/ml, or 1,000 mg/ml of the compound.

In any of the methods, and any aspects of the foregoing, a compound described herein is in the form of a pharmaceutically acceptable salt, solvate, or hydrate. Such a compound may be formulated as a pharmaceutically acceptable salt, e.g., acid addition salt, and complexes thereof. The preparation of such salts can facilitate the pharmacological use by altering the physical characteristics of the agent without preventing its physiological effect. Examples of useful alterations in physical properties include, but are not limited to, increasing the solubility to facilitate administering higher concentrations of the compound.

In any of the methods, and any aspects of the foregoing, a compound or an extract described herein is administered topically, intravenously, intraperitoneally, parenterally, intramuscularly, orally or via the respiratory tract. In any of the methods, and any aspects of the foregoing, the compound or the extract is administered topically.

The compound or an extract described herein may be administered locally, e.g., at a local site of the disease or condition. The compound or an extract described herein may be formulated for local administration, e.g., to a target site of the disease or condition. The compound or an extract described herein may be administered systemically. Systemic administration may be, e.g., topical, intravenous, intraperitoneal, parenteral, intramuscular, oral, or via the respiratory tract. The compound or an extract described herein may be formulated for systemic administration.

In any of the methods, and any of the aspects of the foregoing, the subject animal is a vertebrate. In any of the methods, and any aspects of the foregoing, the subject is a mammal. In any of the methods, and any aspects of the foregoing, the subject is a human. In any of the methods, and any of aspects of the foregoing, the subject is a non-human mammal, e.g., a rodent, e.g., a mouse.

In some aspects, the subject may be female. In some aspects, the subject may be male. The subject may be characterized as one of the following ethnicity/race: Asian, Black or African American, Hispanic or Latino, white, or multi-racial. The subject may be of an age less than 1, or between 1-5, 5-10, 10-20, 20-30, 30-40, 40-50, 50-60, or over 60 years. The subject may suffer from or have been diagnosed with a condition that causes increased risk of severe illness due to a viral infection, or for which a standard of care treatment causes increased risk of severe illness due to a viral infection. Such conditions include, for example, cancer, chronic kidney disease, chronic lung disease (such as chronic obstructive pulmonary disease (COPD), asthma, interstitial lung disease, cystic fibrosis, and pulmonary hypertension), dementia or other neurological condition, diabetes type 1 or type 2, down syndrome, heart conditions (such as heart failure, coronary artery disease, cardiomyopathies, or hypertension), immunocompromised state, liver disease, overweight (for example, having a body mass index of 25 or greater), obesity (for example, having a body mass index of 30 or greater), pregnancy, sickle cell disease or thalassemia, being a current or former smoker, having received a solid organ or blood stem cell transplant, stroke or cerebrovascular disease, substance use disorders, a concurrent viral infection, such as HIV infection, SARS-CoV-2 infection, influenza infection, or MERS infection, or any indication identified by the U.S. Center for Disease Control and Prevention (CDC) as increasing risk of severe illness or for which standard of care causes increased risk of severe illness due to viral infection.

In any of the methods, and any aspects of the foregoing, the compound or the extract is administered in an effective amount. Suitable effective amounts are described in more detail herein. In any of the methods, and any of the aspects of the foregoing, the compound or the extract is administered in a therapeutically effective amount. In any of the methods, and any aspects of the foregoing, the administering step may comprise administering a single dose of a compound or extract according to a course of treatment (where the dose may contain an effective amount). In any of the methods, and any aspects of the foregoing, the administering step may comprise administering more than one dose of a compound or extract according to a course of treatment (where one or more doses may contain an effective amount). The amount of a compound or extract in each dose administered during a course of treatment is not required to be the same. For example, the administering step may comprise administering at least one loading dose and at least one maintenance dose during a course of treatment. Dosing is described in more details herein.

The compound may be administered as a prophylactic treatment. The compound may be administered as a cosmetic or therapeutic treatment.

The compounds disclosed herein may be used in various applications, e.g., cosmetic and/or therapeutic applications. The compounds may be administered in an effective amount for an intended use, e.g., a cosmetic or a therapeutic application. In some embodiments, a composition may comprise a concentration or amount, e.g., an effective amount, of the compound sufficient to have a desired cosmetic effect. In some embodiments, a composition may comprise a concentration or amount, e.g., an effective amount, of the compound sufficient to have a desired therapeutic effect.

An amount and/or frequency of administration may be sufficient to induce mitochondrial biogenesis. An amount and/or a frequency of administration may be sufficient to treat, inhibit, or prevent the progression of at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. An amount and/or frequency of administration may be sufficient to modify a score of a parameter on a qualitative scale, as graded by the subject or a clinical grader. The qualitative scale may comprise the following categories: none (best possible condition), mild, moderate, severe (worst possible condition). The qualitative scale may refer to perceived or actual energy levels and/or vitality.

In some aspects, administering an effective amount of the compound may limit or inhibit at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. For example, the effective amount of the compound may slow progression of the at least one of mitochondrial dysfunction, a disease or condition associated with mitochondrial dysfunction or a symptom thereof, an aging-associated chronic condition associated with mitochondrial dysfunction or a symptom thereof, and/or a decrease in energy level or vitality. In some embodiments, administering an effective amount of the compound may promote mitochondrial biogenesis, mitochondrial function, and/or an increase in energy level or vitality.

In some aspects, administering an effective amount of the compound may increase expression of at least one protein selected from PGC-1a, TFAM, NRF-1, and COX II. Administering an effective amount may decrease expression or inhibit an increase of expression of at least one protein selected from FGF-21 and IL-6, or any cytokine related to viral infection. Administering an effective amount may increase or inhibit a decrease in at least one of ATP-linked respiration, maximal respiration and reserve capacity in subjects infected with SARS-CoV-2. Administering an effective amount may modulate, e.g., increases or decreases, viral protein interaction with one or more host mitochondrial genes, e.g., MRPS2, MRPS5, MRPS25, MRPS27, NDUFAF1, NDUFB9, NDUFAF2, ATP1B1, ATP6V1A, ACADM, AASS, PMPCB, PITRM1, COQ8B, PMPCA, and Tomm70. Administering an effective amount may increase expression of ACE2.

The amount of the compound or composition may be effective in treating or preventing a viral infection in the subject. In certain embodiments, administering an effective amount, e.g., administering a therapeutically or cosmetically effective amount, may decrease the release of viral vesicles from dysfunctional mitochondria. For instance, in some embodiments, administering an effective amount may decrease or inhibit an increase in circulating mtDNA levels, e.g., plasma mtDNA and/or cytoplasmic mtDNA.

In some aspects, the compound may be administered prior to onset of the disease or condition in the subject. The compound may be administered during incidence of the disease or condition in the subject. The compound may be administered subsequent to at least partial reduction of the disease or condition in the subject.

The compound may be administered in response to a trigger or warning sign of a viral infection, e.g., a viral infection-induced symptom, such as a respiratory symptom, gastrointestinal symptom, cardiac symptom, neurological symptom, and/or loss of energy or vitality, e.g., muscle or body aches, fatigue, shortness of breath, difficulty breathing, fever or chills, headache, loss of taste or smell, sore throat, congestion or runny nose, nausea or vomiting, diarrhea, cough, lymphadenitis, rash, or sleep hyperhidrosis.

The compound may be administered in response to a trigger or warning sign of a mitochondrial dysfunction or aging-associated condition, e.g., aging, premature aging, habitual sleep conditions, weight loss, ultraviolet (UV) light exposure, treatment with a chemical or therapeutic agent, e.g., chemotherapy and/or radiation therapy, smoking, dehydration, or immersion. The subject may be predisposed for a mitochondrial dysfunction condition, e.g., based on age, race, skin type, eye color, habit, or heredity.

A method may further comprise determining whether the subject is in need of treatment.

The composition comprising the compound may additionally comprise a moisturizing agent, deodorizing agent, scent, colorant, insect repellant, cleansing agent, or UV-blocking agent.

The composition may include microspheres or microcapsules.

The composition comprising the compound may additionally comprise an anti-inflammatory agent, a pain management or pain relief agent, a decongesting or expectorant agent, or an anesthetic or counterirritant agent. In some embodiments, the composition comprising the compound may additionally comprise acetaminophen, ibuprofen, bismuth subsalicylate, loperamide, oxymetazoline, phylephrine, psudoephedrine, or hydrocortisone.

The composition may be formulated for immediate release or extended release. The composition may be formulated for controlled or sustained release. For example, the composition may be formulated for sustained release over a period of 6 hours, 12 hours, 18 hours, 24 hours, 36 hours, 48 hours, or more.

The subject may be characterized as having normal mitochondrial function. The subject may be characterized as having reduced mitochondrial function. The subject may be characterized as having an increased level of circulating mtDNA, e.g., plasma mtDNA and/or cytoplasmic mtDNA.

The subject may be characterized as suffering from or having been diagnosed with a condition that causes increased risk of severe illness due to a viral infection, or for which a standard of care treatment causes increased risk of severe illness due to a viral infection. The subject may be characterized as having increased expression of at least one protein selected from FGF-21 and IL-6, or a cytokine related to viral infection. The subject may be characterized as having decreased ATP-linked respiration, maximal respiration, or reserve capacity. The subject may be characterized as having a disrupted viral protein interaction with one or more host mitochondrial genes, e.g., MRPS2, MRPS5, MRPS25, MRPS27, NDUFAF1, NDUFB9, NDUFAF2, ATP1B1, ATP6V1A, ACADM, AASS, PMPCB, PITRM1, COQ8B, PMPCA, and Tomm70. The subject may be characterized as having decreased expression of ACE2.

The subject may be characterized as experiencing premature aging or a symptom of premature aging. In certain embodiments, the subject may be characterized by one or more of: an inflammatory phenotype in the skin, a change in mitochondrial protein expression, reduced expression of mitochondrial oxidative phosphorylation complexes, reduced stability of mitochondrial oxidative phosphorylation complexes, a decrease in collagen content of the skin, increased epidermal thickness, increased epidermal hyperplasia, acanthosis, hyperkeratosis, increased expression of at least one gene selected from the group consisting of: NF-κB, COX-2, INF-β1, CCL5, MMP1, MMP2, MMP9, MMP13, IGF1R, VEGF, and MRPS5, decreased expression of at least one of gene selected from the ground consisting of TIMP1, KLOTHO, COL1A1, MTCO2, TFAM, and VDAC, and increased inflammatory infiltrate in skin. The subject may be characterized as having decreased expression of at least one protein selected from PGC-1a, TFAM, NRF-1, and COXII.

In some aspects, a method may further comprise administering a second amount of the compound to the subject. The second amount may be administered as a second dose of the same formulation. The second amount may be administered in another formulation. The second amount may be administered in another formulation by the same route of administration, e.g., a topical solution or oil with a shampoo, conditioner, spray, cream, gel, body wash, soap, or lotion. The second amount may be administered by another route of administration, e.g., each amount may independently be administered topically, parenterally, or enterally.

In some aspects, a method may further comprise administering a second compound to the subject. The second compound may be a compound that is a constituent of the same extract. The second compound may be a compound that is a constituent of another extract. The second compound may be a compound having a similarity score of at least 95% with the first compound. The second compound may be administered in the same formulation. The second compound may be administered in another formulation.

The compound may be administered as part of a combination therapy. The method may further comprise administering a second treatment in combination with the compound. The compound may be administered for a period of time prior to initiating the second treatment. The compound may be administered concurrently with the second treatment. The compound may be administered for a period of time subsequent to ceasing the second treatment. The second treatment may be administered via an alternate mode of administration. The second treatment may be a cosmetic and/or therapeutic treatment.

The compound may be administered in combination with a second agent, e.g., cosmetic or therapeutic agent, approved to treat or commonly used to treat the disease or condition or a symptom thereof.

The compound may be administered in combination with standard of care treatment for a viral infection, for example, one or more of SARS, e.g., SARS-CoV-1 or SARS-CoV-2, HIV, Influenza, MERS, or any viral infection related to mitochondrial dysfunction in a subject. The compound may be administered in combination with one or more anti-viral drugs, e.g., remdesivir, abacavir, didanosine, emtricitabine, iamivudine, stavudine, zalcitabine, zidovudine, tenofovir disproval fumarate, peramivir, zanamivir, oseltamivir phosphate, baloxavir marboxil, ribavirin, interferon-α, lopinavir/ritonavir, and convalescent plasma. The compound may be administered in combination with one or more drugs for symptomatic relief, e.g., acetaminophen, ibuprofen, bismuth subsalicylate, loperamide, oxymetazoline, phylephrine, psudoephedrine, or hydrocortisone.

The compound may be administered in combination with caffeine, B vitamins (e.g., B1, B2, B3, B5, B6, B8, B9 and/or B12), vitamin C, iron, magnesium, and/or zinc.

The compound may be administered in combination with UV-blocking agent, moisturizer, sunscreen, wrinkle cream, retinoid, alpha-hydroxy acid, beta-hydroxy acid, squalene, antioxidant, tretinoin, glycosaminoglycan (GAG), lactic acid, malic acid, citric acid, tartaric acid, hydroquinone, kojic acid, L-ascorbic acid, licorice extract. N-acetylglucosamine, niacinamide, squalene, soy, dermal filler or injection. e.g. hyaluronic acid or calcium hydroxylapatite, botulinum toxin, laser resurfacing procedure, ultrasound therapy, chemical peel, e.g. glycolic acid peel, trichloroacetic acid or salicylic acid, or dermabrasion procedure.

The compound may be administered in combination with antioxidant. Exemplary antioxidants include CoQ10, vitamin C, vitamin E, carotenoids, e.g., beta-carotene, minerals, e.g., selenium and manganese, glutathione, lipoic acid, flavonoids, betaflavinoids, phenols, polyphenols, phytoestrogens, mitoquinol mesylate, and ubiquinone.

The fucus compound may be administered in combination with an an emblica extract or a compound constituent of an emblica extract. The fucus compound may be administered in combination with a chebula extract or a compound constituent of a chebula extract. The emblica compound may be administered with a fucus extract or a compound constituent of fucus extract. The emblica compound may be administered with a chebula extract or a compound constituent of a chebula extract. In accordance with certain embodiments, the combination of two or more of an emblica compound, a fucus compound, and a chebula compound may provide synergistic effects in the treatment of the disease or condition.

In accordance with one or more embodiments, an effective amount of the compound may be administered to a face of a subject. In accordance with one or more embodiments, the compound may be administered to the scalp of the subject. In accordance with one or more embodiments, the compound may be administered to the body of the subject. For example, the compound may be applied to one or more of the more of the forehead, eye region, neck, scalp, head, shoulder, arm, hands, leg, underarm, torso, chest, feet, knee, ankle, back, buttock, or genitals of the subject.

In accordance with one or more embodiments, an effective amount of the compound may be administered enterally or parenterally.

### Dosage and Administration

In accordance with the methods, the compounds of the disclosure are administered to the subject (or are contacted with cells of the subject) in an effective amount.

In certain embodiments, therapeutically the effective amount of a compound of the disclosure ranges from about 0.05 mg/kg/day to about 50 mg/kg/day. In certain embodiments, the effective amount ranges from about 0.05 mg/kg/day to about 40 mg/kg/day. In certain embodiments, the effective amount ranges from about 0.05 mg/kg/day to about 30 mg/kg/day. In certain embodiments, the effective amount ranges from about 0.05 mg/kg/day to about 20 mg/kg/day. In certain embodiments, the effective amount ranges from about 0.05 mg/kg/day to about 10 mg/kg/day. In certain embodiments, the effective amount ranges from about 0.05 mg/kg/day to about 8 mg/kg/day. In certain embodiments, the effective amount ranges from about 0.05 mg/kg/day to about 6 mg/kg/day. In certain embodiments, the effective amount ranges from about 0.05 mg/kg/day to about 4 mg/kg/day. In certain embodiments, the effective amount ranges from about 0.05 mg/kg/day to about 3 mg/kg/day. In certain embodiments, the effective amount ranges from about 0.05 mg/kg/day to about 2 mg/kg/day. In certain embodiments, the effective amount ranges from about 0.05 mg/kg/day to about 1 mg/kg/day. In certain embodiments, the effective amount ranges from about 0.05 mg/kg/day to about 0.8 mg/kg/day. In certain embodiments, the effective amount ranges from about 0.05 mg/kg/day to about 0.6 mg/kg/day. In certain embodiments, the effective amount ranges from about 0.05 mg/kg/day to about 0.4 mg/kg/day. In certain embodiment, the amounts per day described above are administered according to a course of treatment and may be administered in a single dose or in more than 1 dose per day. The amounts per day described above may be administered according to a course of treatment and administered in one dose (q.d.) or two doses each day (b.i.d.), wherein the amount of the compound of the disclosure in each dose need not be the same.

In certain embodiments, each dose is administered according to a course of treatment. As used herein, the term “dose” refers to an amount of a compound of the disclosure administered at a given time point according to a course of treatment. For example, if a course of treatment for a compound of the disclosure is b.i.d (2 times/administrations per day) for 7 days, the two administrations on each of days 1-7 would each comprise administering a dose of a compound of the disclosure (for 2 doses each day). In certain embodiments, a dose is administered q.d. (1 time/administration per day) according to a course of treatment. In certain embodiments, a dose is administered b.i.d. according to a course of treatment. In certain embodiments, a dose is administered t.i.d. (three times/administrations per day) according to a course of treatment.

When 2 or more doses are administered on a given day according to a course of treatment, each dose administered according to the course of treatment may contain the same amount of a compound of the disclosure or one or more of doses administered according to the course of treatment may contain a greater or lesser amount of a compound of the disclosure as compared to another dose administered according to the course of treatment. For example, if a course of treatment for a compound of the disclosure is b.i.d for 7 days, the first dose administered on day 1 may contain a first amount (i.e., 2 mg/kg) and the second dose administered on day 1 may contain a second amount (i.e., 0.5 mg/kg). As another example, if a course of treatment for a compound of the disclosure is b.i.d for 7 days, the first dose administered on day 1 may contain a first amount (i.e., 2 mg/kg), the second dose administered on day 1 may contain a second amount (i.e., 0.5 mg/kg), the two doses administered on each of days 2-4 may contain the second amount, and the two doses administered on each of days 5-7 may contain a third amount (i.e., 1 mg/kg).

A dose may be further divided into a sub-dose. Any given dose may be delivered in a single unit dose form or more than one unit dose form. For example, a dose when given by IV administration may be provided as a single IV infusion (i.e., a single 5 mg/kg IV infusion) or as two or more IV infusions administered one after the other (i.e., two 2.5 mg/kg IV infusions). Further, a sub-dose might be, for example, a number of discrete loosely spaced administrations, such as multiple inhalations from an insufflator, by application of a plurality of drops into the eye, or multiple tablets for oral administration.

In certain embodiments, more than one dose of a compound of the disclosure is administered during a course of treatment. Therefore, in the methods described herein, the methods may comprise the administration of multiple doses during the course of treatment. In certain embodiments, the course of treatment may range from months to years. In certain embodiments, the course of treatment may range from 2 days to 1 month, from 2 days to 3 weeks, from 2 days to 2 weeks, or from 2 days to 1 week. In certain embodiments, the course of treatment may range from 1 year to 20 years or longer. In certain embodiments, a dose is delivered at least 1 time per day (i.e., 1 to 3 times) during the course of treatment. In certain embodiments, a dose is not administered every day during the course of treatment (for example, a dose is be administered at least 1 timer per day every other day, every third day, every week, or every month during the course of treatment). Furthermore, the amount of a compound of the disclosure in each dose need not be the same as discussed above. In certain embodiments, of the foregoing, one or more doses, or all of the doses, contain an effective amount of a compound of the disclosure.

In one embodiment, a course of treatment may comprise administering at least one dose as a loading dose and at least one dose as a maintenance dose, wherein the loading dose contains a greater amount of a compound of the disclosure as compared to the maintenance dose (such as, but not limited to, 2 to 10 times higher). In one aspect of this embodiment, the loading dose is administered initially, either as a single administration or more than one administration, followed by administration of one or more maintenance doses through the remaining course of treatment. For example, for a course of treatment that is q.d. every week for 10 years, a loading dose of 3 mg/kg may be administered as the first dose on week 1 of the course of treatment, followed by maintenance doses of 0.5 mg/kg every week for the remainder of the course of treatment. Furthermore, a loading dose may be given as a dose that is not the first dose administered during a course of treatment. For example, a loading dose may be administered as the first dose on week and as a dose on one or more additional weeks (for example, weeks 10 and 20).

In one embodiment, a course of treatment may comprise administering a first dose formulated in a first composition and administering at least one second or subsequent dose formulated in a second composition. The first composition and the second composition may be the same formulation. The first composition and the second composition may be different formulations.

Pharmaceutical, Cosmetic, and/or Dietary Compositions

Pharmaceutical compositions are provided that comprise an amount, e.g., an effective amount, of a compound of the disclosure. In one embodiment, such pharmaceutical compositions contain a therapeutically effective amount of a compound of the disclosure. In addition, other active agents may be included in such pharmaceutical compositions. Additional active agents to be included may be selected based on the disease or condition to be treated.

The pharmaceutical compositions disclosed may comprise one or more compound of the disclosure, alone or in combination with additional active agents, in combination with a pharmaceutically acceptable carrier and/or excipient and/or in combination with a cosmetically acceptable carrier and/or excipient. Such pharmaceutical compositions may be used in the manufacture of a medicament for use in the methods described herein. The compounds of the disclosure are useful in both free form and in the pharmaceutically acceptable forms, such as pharmaceutically acceptable salts.

The pharmaceutically acceptable carriers and/or excipients and/or cosmetically acceptable carriers and/or excipients are well-known to those who are skilled in the art. The choice of carrier and/or excipient will be determined in part by the particular compound(s), as well as by the particular method used to administer the compound composition. Accordingly, there is a wide variety of suitable formulations of the composition of the disclosure. The following methods and excipients are merely exemplary and are in no way limiting. Suitable carriers and excipients include solvents such as water, alcohol, and propylene glycol, solid absorbants and diluents, surface active agents, suspending agent, tableting binders, lubricants, flavors, and coloring agents. The pharmaceutically and/or cosmetically acceptable carriers can include polymers and polymer matrices. Examples of acceptable carriers include carboxymethyl cellulose, crystalline cellulose, glycerin, gum arabic, lactose, magnesium stearate, methyl cellulose, powders, saline, sodium alginate, sucrose, starch, talc and water, among others. Typically, the acceptable carrier is chemically inert to the active agents in the composition and has no detrimental side effects or toxicity under the conditions of use.

Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N+R′R″R′″R″″Y—, in which the R radicals are identical or different optionally hydroxylated hydrocarbon radicals and Y— is an anion of a strong acid, such as halide, sulfate and sulfonate anions; cetyltrimethylammonium bromide is one of the cationic surfactants which can be used, amine salts of formula N+R′R″R′″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

Exemplary pharmaceutically acceptable carriers and/or excipients and/or cosmetically acceptable carriers and/or excipients include water, silica, glycerin, dimethicone, butylene glycol, pentylene glycol, ethoxydiglycol, polyacrylate-13, pentapeptide-34 trifluoroacetate, polyisobutene, lysolecithin, sclerotium gum, pullulan, polysorbate 20, diethylhexyl syringylidenemalonate, caprylyl glycol, glyceryl stearate, PEG-100 stearate, cetearyl alcohol, butyrospermum parkii (shea) butter, acetyl tetrapeptide-2, betaine, melanin, tocopheryl acetate, tocopherol, hydroxyacetophenone, caprylic/capric triglyceride, batyl alcohol, C12-15 alkyl benzoate, panthenol, ceteareth-20, xanthan gum, ethylhexylglycerin, disodium EDTA, propanediol, caprylyl glycol, potassium sorbate, sorbic acid, and phenoxyethanol.

The compounds of the disclosure and pharmaceutical compositions containing such compounds as described in the instant disclosure can be administered by any conventional method available for use in conjunction with pharmaceuticals, either as individual therapeutic agents or in combination with additional therapeutic agents.

In one embodiment, the compounds of the disclosure are administered in an effective amount, whether alone or as a part of a pharmaceutical composition. The effective amount and the dosage administered will, of course, vary depending upon known factors, such as the pharmacodynamic characteristics of the particular agent and its mode and route of administration, the age, health and weight of the recipient; the severity and stage of the disease state or condition; the kind of concurrent treatment; the frequency of treatment; and the effect desired.

The total amount of the compound administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

In these pharmaceutical or cosmetic compositions, the compound(s) of the disclosure will ordinarily be present in an amount of about 0.5-95% weight based on the total weight of the composition, or about 0.1-99.9% weight based on the total weight of the composition. Multiple dosage forms may be administered as part of a single treatment.

The active agent can be administered enterally in solid dosage forms, such as capsules, tablets, and powders, or in liquid dosage forms, such as milk, elixirs, syrups and suspensions. It can also be administered parenterally, in sterile liquid dosage forms. The compound(s) of the disclosure can also be administered intranasally (nose drops) or by inhalation via the pulmonary system, such as by propellant based metered dose inhalers or dry powders inhalation devices. Other dosage forms include topical administration, such as administration transdermally, via patch mechanism or ointment.

Formulations suitable for enteral or oral administration may be liquid solutions, such as an effective amount of the compound(s) dissolved in diluents, such as milk, water, saline, buffered solutions, infant formula, other suitable carriers, or combinations thereof. Formulations suitable for enteral or oral administration of the compounds of the disclosure are known in the art as exemplified by: Shaji, et al., Indian J Pharm Sci. 2008 May-June; 70(3): 269-277; Bruno, et al., Ther Deliv. 2013 November; 4(11): 1443-1467; Ibrahim, et al., DARU Journal of Pharmaceutical Sciences, 2020, 28, 403-416. The compound(s) can then be mixed to the diluent just prior to administration. In an alternate embodiment, formulations suitable for enteral or oral administration may be capsules, sachets, tablets, lozenges, and troches. In each embodiment, the formulation may contain a predetermined amount of the compound(s) of the disclosure, as solids or granules, powders, suspensions and suitable emulsions. Liquid formulations may include diluents, such as water and alcohols, for example, ethanol, benzyl alcohol, propylene glycol, glycerin, and the polyethylene alcohols, either with or without the addition of an acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers.

Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) can be administered in a physiologically acceptable diluent in an acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of an acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl .beta.-aminopropionates, and 2-alkylimidazoline quaternary ammonium salts, and (e) mixtures thereof.

The parenteral formulations typically contain from about 0.5% to about 50% by weight of the compound(s) in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The compound(s) of the disclosure can be formulated into aerosol formulations to be administered via nasal or pulmonary inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer.

The compound(s) of the disclosure, alone or in combination with other suitable components, may be administered in an aqueous solution as a nasal or pulmonary spray and may be dispensed in spray form by a variety of methods known to those skilled in the art. Systems for dispensing liquids as a nasal spray are disclosed in U.S. Pat. No. 4,511,069. The formulations may be presented in multi-dose containers, for example in the sealed dispensing system disclosed in U.S. Pat. No. 4,511,069. Additional aerosol delivery forms may include, e.g., compressed air-, jet-, ultrasonic-, and piezoelectric nebulizers, which deliver the active agent dissolved or suspended in a pharmaceutical solvent, e.g., water, ethanol, or a mixture thereof.

Nasal and pulmonary solutions of the disclosure may typically comprise the drug or drug to be delivered, optionally formulated with a surface-active agent, such as a nonionic surfactant (e.g., polysorbate-80), and one or more buffers. In some embodiments, the nasal spray solution further comprises a propellant. The pH of the nasal spray solution is optionally between about pH 3.0 and 6.0, or 4.5+/−0.5. Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, chlorobutanol, benzylalkonimum chloride, and the like. Suitable surfactants include, but are not limited to, oleic acid, sorbitan trioleate, polysorbates, lecithin, phosphatidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like.

Within alternate embodiments, nasal and pulmonary formulations are administered as dry powder formulations comprising the active agent in a dry, usually lyophilized, form of an appropriate particle size, or within an appropriate particle size range, for intranasal delivery. Minimum particle size appropriate for deposition within the nasal or pulmonary passages is often about 0.5 μm. mass median equivalent aerodynamic diameter (MMEAD), commonly about 1 μm MMEAD, and more typically about 2 μm MMEAD. Maximum particle size appropriate for deposition within the nasal passages is often about 10 μm MMEAD, commonly about 8 μm MMEAD, and more typically about 4 μm MMEAD. Intranasally and pulmonarily respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI), which relies on the patient's breath, upon pulmonary or nasal inhalation, to disperse the power into an aerosolized amount. Alternatively, the dry powder may be administered via air-assisted devices that use an external power source to disperse the powder into an aerosolized amount, e.g., a piston pump.

To formulate compositions for nasal or pulmonary delivery, the active agent can be combined with various pharmaceutically and/or cosmetically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for nasal or pulmonary delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about ⅓ to 3, more typically ½ to 2, and most often ¾ to 1.7.

The compound(s) of the disclosure may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g., maleic anhydride) with other monomers (e.g., methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the active agent.

The compounds of the disclosure may be formulated as described in International Patent Application No. PCT/US2022/020983, filed Mar. 18, 2022, titled “COMPOSITIONS AND METHODS FOR IMPROVING MITOCHONDRIAL FUNCTION,” the entire disclosure of which is herein incorporated by reference in its entirety for all purposes.

The compounds of the disclosure may be formulated in a nanoparticle-based delivery carrier. The nanoparticle delivery systems disclosed herein may offer benefits in mitochondria-targeted delivery and improving the therapeutic ability of the compounds. Nanoparticle formulations have been shown to effectively transport drug molecules in their original form, solubilize hydrophobic drug molecules, enhance half-life of molecules, and decrease side effects and immunogenicity attributable to the molecules. The nanoparticle delivery carriers disclosed herein may be selected or designed to provide enhanced skin penetration, higher stability, site specific targeting, e.g., efficiently deliver cargo inside the mitochondrial matrix, high entrapment efficiency, and/or time-controlled, e.g., delayed or sustained, release of the compounds. Properties of the nanoparticle delivery carrier that may be designed to effectively deliver compounds of the formulation to a target site of a subject include surface chemistry, coating, structure, size, ability to aggregate, and solubility. Exemplary nanomaterials are described in “Nanotherapeutic Approaches to Target Mitochondria in Cancer,” (Mani, 2021) and “Role of Nanotechnology in Cosmeceuticals: A Review of Recent Advances,” (Kaul, 2018), each of which is incorporated herein by reference in its entirety for all purposes.

The nanoparticle delivery carrier may comprise and/or be functionalized with carbon-based nanomaterials, liposomal delivery vehicles, polymeric nanocarriers, micelles, dendrimers, lipophilic cations, solid-lipid nanoparticles (SLN), peptide-based nanomaterials, nanostructured lipid carriers (NLC), niosomes, nanoemulsions, metal nanoparticles, nanospheres, polymerosomes, cubosomes, and combinations thereof. The delivery carrier may be designed to for mitochondrial targeting or specific cell type targeting. Exemplary mitochondrial targeting agents include antibodies, polymeric functional moieties, such as PEG, lipophilic cations, such as triphenylphosphine (TPP), and peptides, such as mitochondrial penetrating peptides (MPP). The delivery carrier may be formed of a targeting moiety, for example, encapsulating or conjugated with the compounds disclosed herein, and/or the delivery carrier may be functionalized with a surface targeting moiety. The nanoparticle carrier may be dimensioned to have an average size of about 10 to 5000 nm, for example 10 to 50 nm, 10 to 100 nm, 50 to 500 nm, 50 to 100 nm, 100 to 500 nm, 500 to 1000 nm, or 1000 nm to 5000 nm, which can be selected based on the target tissue, compound to be delivered, and other properties of the nanomaterial.

Exemplary carbon-based nanomaterials include carbon dots (C-dots or CD), carbon nanotubes (CNT), graphene derivatives, nanodiamonds (ND), quantum dots (QD), and magnetic nanoparticles (MNP). Carbon nanoparticles may be formed into different shapes including, for example, spherical, elliptical, tube, horn-shaped, and combinations thereof.

CNTs are graphene sheets formed into cylindrical tubes. CNTs have demonstrated low toxicity profile, good biocompatibility, and targeted accumulation. CNTs of the disclosure may be single-walled carbon nanotubes (SWCNT) and/or multi-walled carbon nanotubes (MWCNT). SWCNTs have a smaller diameter, in the range of 1 to 10 nm. MWCNTs have a larger diameter, in the range of 2 to 50 nm. CNTs may be functionalized with a targeting sequence. For instance, CNTs may be functionalized to target mitochondria and/or a specific cell type. Graphene derivatives also include two-dimensional carbon allotropes. Graphene derivatives may be designed to exhibit specific physicochemical properties, such as a high surface area and selected multifaceted surface properties. Graphene derivatives may be functionalized, e.g., surface functionalized, for targeted delivery to mitochondria and/or a specific cell type. CNTs have been used successfully in hair colorants and cosmetic hair care formulations.

Nanodiamonds have been shown to provide high affinity to biomolecules, biocompatibility, and non- or low-cytotoxicity. Nanodiamonds, quantum dots, and magnetic nanoparticles may be conjugated with the compounds disclosed herein for targeted delivery. Certain MNP may be designed to encapsulate the compounds disclosed herein. QDs are generally formed of a semiconductive core layered by a shell designed to provide selected physical and chemical characteristics. MNPs are formed of a magnetic core, such as an iron oxide material core, and surface coating designed to improve stability and biocompatibility in physiological environments. Thus, MNPs and QDs are highly adaptable for their selected use.

Liposome based delivery vehicles are typically formed of enclosed spherical vesicles composed of a lipid bilayer with an internal bilayer and internal aqueous core region. The liposome may have a unilamellar or multilamellar structure. Liposomes may be designed to have a surface chemistry effective for targeted delivery and/or controlled release delivery. For instance, engineered liposomes have shown improved cellular update and accumulation of a delivery compound in the mitochondria. Antioxidants, such as carotenoids, CoQ10, lycopene and agents such as vitamin A, E, and K, may be incorporated into liposomes to amplify physical and chemical stability. Liposomes may be formulated with phosphatidylcholine to provide moisturizing properties to skin and hair care products. Vegetable phospholipids and soya phospholipids may be used with topical formulations for their high content of esterified essential fatty acids. For example, when applied with an active agent, the barrier function of the skin is increased and water loss is decreased. It has been shown that certain liposomes have an effect on wrinkle reduction, decreasing efflorescence in acne treatment, and increasing skin smoothness.

Niosomes are vesicles having a bilayer structure composed of self-assembled hydrated nonionic surfactants. Niosomes may have cholesterol incorporated into their lipids or may be free of cholesterol. Niosomes may be formulated as multilamellar or unilamellar structures encapsulating the compounds disclosed herein by a membrane formed when the surfactant macromolecules are organized as a bilayer. Exemplary nonionic surfactants include spans, tweens, brijs, alkyl amides, sorbitan ester, crown ester, polyoxyethylene alkyl ether, and steroid-linked surfactants. Niosomes may encapsulate the compounds disclosed herein, providing prolonged systemic circulation and enhanced penetration into target tissue. Niosomes in cosmetic and skin care applications provide skin penetration, increased stability of entrapped ingredients, and improved bioavailability of typically poorly adsorbed compounds. Niosomes may be designed for a target application by controlling the nature and structure of surfactants, membrane composition, and temperature of hydration, which influences size and shape of the particle. Specialized niosomes called proniosomes may be used. Proniosomes are nonionic based surfactant vesicles which are hydrated immediately before use to yield aqueous noisome dispersions. To further enhance drug delivery, niosomes and proniosomes may be combined in a formulation.

Polymeric nanocarriers are generally formed of biodegradable polymers. The polymeric nanocarriers may be reservoir type (nanocapsules in which compounds are dissolved/distributed in the core of the polymer), matrix type (nanospheres, in which compounds are entrapped in the polymer matrix), and combinations thereof. Polymeric nanocarriers may offer the benefits of low toxicity, easy modification (for example, for targeted delivery), high drug loading capacity, small size, good aqueous solubility, and biocompatibility. Exemplary polymeric nanocarriers for mitochondrial targeting include hydrophilic block polymer, such as polyethylene glycol (PEG), poly E-caprolactone (PCL). Other nanoparticles disclosed herein may be modified for mitochondrial targeting, such as by including surface hydrophilic block polymers (e.g., PEGylation). Polymerosomes are artificial vesicles formed of self-assembling block copolymer amphiphiles. Polymerosomes typically have a hydrophilic core and lipophilic bilayer.

Polymerosomes are highly customizable. Drug encapsulation and release capabilities may be controlled by forming the polymerosome with block copolymers that are biodegradable and/or responsive to stimuli. The composition and molecular weight of the polymerosome may be selected to control properties, such as, response to stimuli, membrane thickness, permeability, flexibility, and size (polymerosomes may be designed to have a radius from 50 nm to 5000 nm or more). Polymerosomes provide benefits, such as, improved skin elasticity and skin cell activation energy enhancement.

Cubosomes are nanostructured particles formed from self-assembled liquid crystalline particles of aqueous lipids and surfactants. Cubosomes are formed of a bicontinuous liquid phase, enclosing two separate vesicles of aqueous formulations divided by a surfactant-controlled bilayer in a strongly packed structure. Cubosomes may be designed as a honeycombed structure having more than two separate vesicles. Release of each vesicle may be separately controlled. Cubosomes may provide benefits, such as, providing controlled and/or targeted release of the compounds, possessing lipid biodegradability, and having a high internal surface area with different drug-loading modalities.

Micelles are colloidal aggregates that are generally amphiphilic in nature, having a hydrophilic head and hydrophobic tail. The size and shape of micelle nanoparticles may be selected by varying the solution's isotonic strength, pH, temperature, and the nature of the amphiphilic molecule. Micelles may be utilized to improve uptake of the compounds disclosed herein in mitochondria. For instance, micelle formulations have been found to improve bioavailability of low absorption compounds, prevent mitochondrial swelling (indicating less mitochondrial permeability transition pore (mPTP) opening and prevention of injury), and protect cells from nitrosative stress, “Curcumin Micelles Improve Mitochondrial Function in Neuronal PC12 Cells and Brains of NMRI Mice—Impact on Bioavailability” (Hagl, 2015) (incorporated herein by reference in its entirety for all purposes). Micelles may be functionalized with a targeting group, such as mitochondrial targeting TPP, MPP, or PEGyltaion. In some embodiments, micelle nanoparticles may be polymeric micelles.

Dendrimers are hyperbranched macromolecules which may be formed of sugars, amino acids, and/or nucleotides. Dendrimers are generally formed of a central core, repeated branches, and diverse peripheral groups. The peripheral groups may be designed or functionalized for a target application, such as targeted delivery. Benefits of dendrimers include the ability to provide drug encapsulation, high aqueous solubility, high retention time, biodegradability, specificity, low toxicity, and surface modification capabilities that may provide properties such as monodispersity, polyvalence, and stability. Dendrimers may be functionalized with a targeting group, such as TPP, MPP, or PEGylation. Dendrimers may be formulated to encapsulate the compound or conjugate to the compound.

Lipophilic cations are positively charged ions that can penetrate the plasma and mitochondrial membranes. The lipophilic cations tend to accumulate in the mitochondria. Thus, lipophilic cations, such as TPP, dequalinum, and rhodamine 123, may be utilized for mitochondrial-targeted therapeutic delivery. Delocalized lipophilic cations (DLC) have a strong mitochondrial targeting ability to cross the membrane and drive specific aggregation of attached moieties within the mitochondria of the cells. Thus, DLCs may be utilized as nanoparticle carriers and/or as surface modifications for targeted delivery. For instance, the compositions disclosed herein may be conjugated to a DLC, such as TPP, or encapsulated in another carrier having a TPP surface functionalization for targeted delivery.

Solid-lipid nanoparticles are sub-micron colloidal carriers in the range of 50 to 1000 nm, for example 50 to 500 nm or 50 to 100 nm. SLNs are generally formed of physiological lipid disseminated in water or a liquid surfactant solution having an oil-based or lipoidal core. SLNs may be prepared from complex glyceride mixtures, purified triglycerides, and waxes having phospholipid hydrophobic chains in the fat matrix. SLNs provide benefits such as small size, large surface area, high drug loading capacity, and the contact of phases at the interface. SLNs may be designed to provide controlled or sustained release of the compound. In cosmetics and pharmaceuticals, SLNs may provide increased penetration of the compounds disclosed herein through the skin. SLNs may have ultraviolet (UV) resistant properties, occlusive properties which can be used to increase skin hydration, and good stability coalescence due to their solid nature, which reduces mobility and leakage of the active molecules.

Nanostructured lipid carriers (NLC) are a form of lipid nanoparticle formed by blending solid lipids with spatially incompatible liquid lipid compositions, forming an amorphous solid. NLCs may be of the imperfect type, amorphous type, or multiple type. NLC particles typically range in size from 10 nm to 1000 nm. When formulated from biodegradable and physiological lipids, NLCs show very little toxicity. Thus, NLC formulations may provide the benefits of reduced systemic side effects and higher drug loading capacity. NLCs may be designed to have a biphasic drug release pattern. For example, a first compound release profile may be immediate or controlled release, and a second compound release profile may be controlled or delayed release. Like SLNs, NLCs may also provide increased penetration of the compounds disclosed herein through the skin, ultraviolet (UV) resistant properties, occlusive properties which can be used to increase skin hydration, and good stability coalescence.

Nanoemulsions are kinetically and thermodynamically stable dispersions of liquid formed from an oil phase and a water phase in combination with a surfactant. The nanoemulsions disclosed herein may be oil in water, water in oil, or bicontinuous formulations. Properties of nanoemulsions may be designed by controlling method of preparation. Nanoemulsions are typically dispersed phase, comprising small particles or droplets having low oil or water interfacial tension. Nanoemulsions are typically formed of a lipophilic core surrounded by a monomolecular layer of phospholipids. Nanoemulsions provide benefits, such as, low viscosity, high kinetic stability, high interfacial area, high solubilization capacity, and increased rate of absorption. In cosmetic and pharmaceuticals, nanoemulsions may provide rapid penetration and active transport of active ingredients and hydration to the skin. Nanoemulsions may be formulated into foams, creams, sprays, or liquids.

Certain nanoparticle formulations, such as SLNs, nanoemulsions, liposomes, and niosomes, may be used in moisturizing formulations, providing humectants that retain moisture for a prolonged period of time.

Metal nanoparticles may be designed to have specific properties and may be shaped as nanospheres, nanoshells, nanoclusters, nanorods, nanostars, nanocubes, branched, and nanotriangles. Shape, size, and dielectric properties of metal nanoparticles may have an effect on resonance frequency. Metal nanoparticles may be designed to have high drug loading capacity and effectively penetrate the cell wall by controlling size, surface area, and crystallinity. Benefits of metal nanoparticles include acceleration of blood circulation, anti-inflammatory properties, antiseptic properties, improvising firmness and elasticity of skin, delaying aging, and vitalizing skin metabolism. Exemplary metal nanoparticles are gold, silver, and copper. Such metal nanoparticles have been shown to provide strong antifungal and/or antimicrobial properties. Another exemplary metal nanoparticle is titanium dioxide (TiO₂), which has been shown to provide protection from ultraviolet (UV) radiation. Metal nanoparticles may be designed to be inert in nature, highly stable, biocompatible, and noncytotoxic. More than one metal may be used to form metal nanocomposites with selected properties.

Nanospheres are spherical nanoparticles having a core-shell structure. The compounds may be encapsulated, conjugated, dissolved, or otherwise entrapped in the nanoparticle. The nanospheres may be crystalline or amorphous structures. The nanospheres may be biodegradable or nonbiodegradable. Exemplary biodegradable biospheres include gelatin, modified starch, and albumin nanospheres. One exemplary nonbiodegradable nanosphere is polylactic acid. In cosmetics and pharmaceuticals, nanospheres may be used to deliver the compounds disclosed herein into a deep layer of the skin more precisely and efficiently. Nanospheres have been shown to provide protection against actinic aging.

Peptide-based nanomaterials are biomolecules made up of several amino acids linked by peptide bond. Peptides may generally provide rapid clearance in the kidney due to enzymatic degradation. Peptide nanomaterials may provide several benefits including targeting and accumulating capacity, small size, ease of production and customizability, and biocompatibility. Certain peptide nanomaterials may be designed to self-assemble into distinct shapes and sizes in response to environmental factors, such as temperature, pH, ionic strength, or molecular interaction between the host and peptide. Peptide nanoparticles may also be functionalized for targeted delivery. Functionalized peptides have been found to exhibit improved targetability and enhanced efficacy.

One exemplary peptide nanomaterial with mitochondrial-targeting ability is mitochondrial penetrating peptides (MPP). MPPs are cell penetrating peptides which can efficiently penetrate mitochondrial double membranes. MPPs are generally designed or selected to be positively charged peptides. Due to the strongly negative charge of mitochondrial membranes, positively charged peptides are capable of penetrating mitochondria. MPPs are described in more detail in “Mitochondrial targeted strategies and their application for cancer and other diseases treatment,” (Li, 2020), incorporated herein by reference in its entirety for all purposes. Thus, MPPs may be utilized as nanoparticle carriers and/or as surface modifications for targeted delivery. For instance, the compositions disclosed herein may be conjugated to an MPP or encapsulated in another carrier having an MPP surface functionalization for targeted delivery.

The formulations disclosed herein may comprise one or more skin penetration enhancer. Generally, small and moderately lipophilic molecules are likely to penetrate the skin barrier. Other compounds may require a suitable skin penetration enhancer to penetrate the barrier by either diminishing the barrier properties of the skin or actively driving movement of compounds across the skin with the input of external energy. Skin penetration enhancers are described in “Penetration Enhancement of Topical Formulations,” (Ng, 2018) and “Transdermal Delivery Systems in Cosmetics,” (Kim, 2020), each of which is incorporated herein by reference in its entirety for all purposes.

The nanoparticle delivery carriers disclosed herein may provide skin penetration enhancement for the compounds. The formulations disclosed herein may additionally or alternatively contain one or more chemical or physical skin penetration enhancers. Exemplary chemical skin penetration enhancers include alcohols, such as, ethanol and glycol, sulfoxides, such as, dimethyl sulfoxide, laurocapram, pyrrolidones, dimethyl isosorbide, isopropyl myristate, propylene glycol, oleic acid, eucalyptol, water/aqua (hydration), surfactants, urea, fatty acids, fatty alcohols, and terpenes and/or terpenoids. Exemplary physical skin penetration enhancers include rollers, scrapers, scrubbers, exfoliators, microdermabrasion needles, iontophoresis devices, electroporation devices, ultrasound devices, such as sonophoresis, thermal ablation, magnetophoresis, photomechanical waves, electron beam irradiation, and low light therapy devices, such as light emitting diode (LED) sources. Two or more skin penetration enhancers may be used in the formulation synergistically.

Chemical skin penetration enhancers may be included in dermatological, transdermal, cosmetic, and pharmaceutical products to enhance the dermal absorption of drug compounds. Chemical enhancers may enable or improve solubility, penetration, and/or absorption of the compounds disclosed herein. Exemplary chemical enhancers are described below.

Water (aqua) generally increases fluidity of the composition, providing higher permeability. Additionally, water hydrates the skin barrier, altering skin lipids and/or proteins for improved permeation. Hydrating compounds, such as glycerol and urea, may promote transdermal permeation by facilitating hydration of the stratum corneum and forming hydrophilic diffusion channels within the barrier.

Alcohol solvents, such as ethanol and propylene glycol, may provide penetration enhancement by increasing fluidity of the compound and also act as good solvents. Degree of permeation may be selected by controlling alkyl chain length of fatty alcohols.

Surfactants generally solubilize lipophilic agents, including active ingredients of the formulation as well as lipids within the stratum corneum. Thus, surfactants may enhance skin permeability by partitioning into the epithelial cell membranes and disrupting the packing of membrane lipids, forming structural defects that reduce membrane integrity. The effect of the surfactant on skin permeation may be designed by selecting concentration and type of surfactant. The surfactant may be anionic, cationic, or nonionic. Anionic surfactants may be selected for skin or hair applications because they interact with keratin and lipids. Cationic surfactants may be selected for skin applications because they interact with skin proteins via poler interactions. Non-ionic surfactants are generally less irritating to the skin and have better tolerability.

Fatty acids may increase percutaneous drug absorption. Long-chain fatty acids, which are carboxylic acids with typically long, unbranched aliphatic tails, have been demonstrated to increase percutaneous drug absorption as an effect of alkyl chain length. Low molecular weight alkanols may act as solubilizers to enhance the solubility of the compound in the fatty matric of the stratum corneum. Polyunsaturated fatty acids, such as linoleic, alpha-linoleic, and arachidonic acids, may enhance the skin permeation. One exemplary fatty acid chemical penetration enhancer is oleic acid. Oleic acid provides increased fluidity and reduced resistance toward the permeation of molecules.

Terpenes are hydrocarbons commonly found in plant extracts. Terpenoids are terpenes containing additional functional groups. One group of terpenes that may provide penetration enhancement are oxygen-containing terpenes. Exemplary oxygen containing terpenes include menthol, thymol, carvacrol, menthone, and cineole. Such terpenes may enhance penetration in a similar mechanism as alcohols. However, terpenes may be considered natural products. Benefits of terpenes include high percutaneous ability with minimal irritancy and toxicity.

The compounds of the disclosure may be formulated with a mitochondria-targeting agent. Exemplary mitochondrial targeting agents include antibodies, polymeric functional moieties, such as PEG, lipophilic cations, such as triphenylphosphine (TPP), and peptides such as mitochondrial targeting peptides (MPP).

The compounds of the disclosure may alternatively contain as pharmaceutically and/or cosmetically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically and/or cosmetically acceptable carriers can be used which include, for example, pharmaceutical grades of mannitol, lactose, starch, magnesium stearate, sodium saccharin, talcum, magnesium carbonate, and the like.

Compositions of the disclosure can also be formulated as a solution, microemulsion, or other ordered structure suitable for high concentration of active ingredients. The carrier can be a solvent or dispersion medium containing, for example, water, ethanol, polyol (for example, glycerol, propylene glycol, and liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants.

In certain embodiments, compound(s) and compositions of the disclosure are administered in a time-release formulation, for example in a composition which includes a slow release polymer. Such compositions can be prepared with carriers that will protect against rapid release, for example a controlled release vehicle such as a polymer, microencapsulated delivery system or bioadhesive gel. Prolonged delivery, in various compositions of the invention can be brought about by including in the composition agents that delay absorption, for example, aluminum monosterate hydrogels and gelatin. When controlled release formulations is desired, controlled release binders suitable for use in accordance with the invention include any biocompatible controlled-release material which is inert to the active agent and which is capable of incorporating the biologically active agent. Numerous such materials are known in the art.

Formulations suitable for topical administration include solutions, oils, creams, emulsions, and gels containing, in addition to the active ingredient, such carriers as are known in the art. Topical formulations may be pharmaceutical or cosmetic formulations. In some embodiments, the compound is formulated as a shampoo, conditioner, spray, cream, gel, balm, body wash, soap, lotion, or make-up.

The compounds of the disclosure and compositions of the disclosure can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Suitable unit doses, i.e., effective amounts, may be determined during clinical trials designed appropriately for each of the conditions for which administration of a chosen compound is indicated and will, of course, vary depending on the desired clinical endpoint. Extemporaneous injection solutions and suspensions can be prepared from sterile powders, granules, and tablets. The requirements for effective pharmaceutically acceptable carriers for injectable compositions are well known to those of ordinary skill in the art. See Pharmaceutics and Pharmacy Practice, J.B. Lippincott Co., Philadelphia, Pa., Banker and Chalmers, Eds., 238-250 (1982) and ASHP Handbook on Injectable Drugs, Toissel, 4^{th} ed., 622-630 (1986).

Additionally, formulations suitable for rectal administration may be presented as suppositories by mixing with a variety of bases such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

The compositions disclosed herein may be associated with a variety of natural products, and examples of such products are set out below. The compositions disclosed herein may be formulated as a natural product. The compositions disclosed herein may be administered in combination with a natural product. The compositions disclosed herein may be incorporated in a natural product. These natural products may be comprised of formulations or compositions disclosed throughout this disclosure.

Natural products may be or comprise products for commercial purposes, and may refer to dietary supplements, and foods, e.g., food, food supplements, medical food, food additive, nutraceutical, or drink, produced from natural sources. Natural products may have pharmacological or biological activity that may be of therapeutic benefit, e.g., in treating disease or conditions. Natural products may be included in traditional medicines, treatments for cosmetological purposes, cosmetics, and spa treatments. A natural product referred to herein may comprise any one or more of the components described as a natural product to be incorporated into a composition or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

One skilled in the art will appreciate that suitable methods of administering a compound of the disclosure to a patient are available, and, although more than one route can be used to administer a particular compound, a particular route can provide a more immediate and more effective reaction than another route.

### EXAMPLES

The function and advantages of these and other embodiments can be better understood from the following examples. These examples are intended to be illustrative in nature and are not considered to be limiting the scope of the invention.

### Example 1: Decoding SARS-CoV-2 Hijacking of Host Mitochondria in COVID-19 Pathogenesis

Without wishing to be bound by theory, it is hypothesized that the SARS-CoV-2 virus localization of RNA transcripts in mitochondria hijacks the host cell's mitochondrial function to viral advantage. The hypothesis was based on available data for the SARS-CoV-1 virus.

Besides viral RNA transcripts, RNA also localizes to mitochondria. It is hypothesized that SARS-CoV-2 may manipulate mitochondrial function indirectly, first by ACE2 regulation of mitochondrial function, and once it enters the host cell, open-reading frames (ORFs) such as ORF-9b may directly manipulate mitochondrial function to evade host cell immunity and facilitate virus replication and COVID-19 disease. Manipulations of host mitochondria by viral ORFs can release mitochondrial DNA (mtDNA) in the cytoplasm and activate mtDNA-induced inflammasome and suppress innate and adaptive immunity.

It is believed that a decline in ACE2 function in aged individuals, coupled with the age-associated decline in mitochondrial functions resulting in chronic metabolic disorders like diabetes or cancer, may make the host more vulnerable to infection and health complications to mortality. These observations suggest that distinct localization of viral RNA and proteins in mitochondria may play essential roles in SARS-CoV-2 pathogenesis. Understanding the mechanisms underlying virus communication with host mitochondria may provide critical insights into COVID-19 pathologies.

Both SARS-CoV-1 and CoV-2 enter into the cell by viral spike (S) proteins binding to cellular receptors and on S protein priming by host cell proteases. Recently, it was demonstrated that like SARS-CoV-1, SARS-CoV-2 transmembrane serine protease 2 (TMPRSS2) primes S protein and uses angiotensin converting enzyme carboxypeptidase 2 (ACE2) host receptor for entry into the cell. Genetic variation analyses in the ACE2 gene showed substantial variation among populations worldwide. Thus, ACE2 might impact CoV-2 entry differently in different people. Interestingly, ACE2 variation may also affect mitochondrial function, a mechanism that CoV-2 may use to preferentially infect populations compromised in mitochondrial function due to either variation in ACE2 or chronic diseases such as diabetes in which mitochondrial dysfunction plays a significant role.

To identify variations among the ORFs encoded by SARS-CoV-1 and SARS-CoV-2, we analyzed genome diversity between the two coronaviruses from different regions of the world. We report a lack of significant mutational differences between the CoV-1 and CoV-2 and highlight how CoV-2 targeting of host mitochondria by direct transport of viral RNA as well as the RNA transcripts hijacks and manipulates mitochondrial function to facilitate host immune suppression, viral replication, and COVID-19.

SARS-CoV-1 entry into the cell depends on angiotensin converting enzyme carboxypeptidase 2 (ACE2) and transmembrane serine protease 2 (TMPRSS2). It has been demonstrated that SARS-CoV-2 also uses the host ACE2 for entry and the TMPRSS2 for S protein priming. SARS-CoV-2 uses ACE2 as the entry receptor. ACE2 utilization serves as a critical determinant of CoV-2 transmissibility. The ACE2 gene is present in the X-chromosome of humans.

The ACE2, which cleaves angiotensin II (Ang II) into angiotensin 1-7 (Ang 1-7), regulates mitochondrial functions. ACE2-knockout mice exhibit impaired mitochondrial respiration and reduced production of ATP. ACE2 overexpression also restores impaired mitochondrial function. It also regulates mitochondria-localized NADPH oxidase 4, which is known to produce reactive oxygen species (ROS) in the mitochondria.

We checked the spatial variations present in the ACE2 gene to see whether some populations carry substitutions that categorize them as more susceptible or resistant to the virus. Consistent with previous studies, we didn't find any rare variant in the coding region; however, an exonic variant rs2285666 (G8790A) showed a significant difference (P=2×10\hat{ }16) for many populations. It is likely that a high rate of rs2285666 allele among populations may modulate the susceptibility for SARS-CoV-2. The pathogenicity of these variants was studied extensively for type 2 diabetes, coronary artery disease, and hypertension. It is interesting to note that the G/G genotype could reduce the expression of ACE2 protein up to 50%.

Besides ACE2, TMPRSS2 transmembrane serine protease also allows the entry of coronaviruses into host cells by proteolytic cleaving and activating viral envelope glycoproteins. Among the coronaviruses, HCoV-229E, MERS-CoV, SARS-CoV, and SARS-CoV-2 are demonstrated to be dependent on TMPRSS2. Indirect evidence suggests that TMPRSS2 may regulate mitochondrial function via ERRa (estrogen-related receptor-a). ERRa is a ligand-independent nuclear receptor that, together with its coactivator peroxisome proliferator-activated receptor-y coactivator-1a (PGC-1a), transcriptionally regulates energy homeostasis and mitochondrial functions. Alternatively, spliced transcript variants of TMPRSS2 isoforms have been described. It is conceivable that an isoform(s) may localize into the mitochondria and play a direct role in regulating mitochondrial function.

Recent evidence indicates that mitochondria play a central role in the host response upon viral infection and immunity. Mitochondrial stress induces the formation of mitochondria-derived vesicles that are involved in the cross-talk between mitochondria and endoplasmic reticulum (ER). Coronavirus replication consists of the formation of double-membrane vesicles (DMVs) derived from ER. These DMVs serve as a site for viral replication and help conceal the virus from host cellular defenses. Like viral manipulation of ER, it is theorized that CoV-2 manipulation of mitochondria results in the induction of (double-membrane) mitochondria-derived vesicles (MDVs). Thus, it is believed that CoV-2 RNA localization in mitochondria may induce mitochondrial dysfunction and increase mitochondria-derived double-membrane vehicles in which the virus can hide and replicate. In this context, it noteworthy that point mutations in the murine coronavirus are known to decrease the number of ER-derived DMV but simultaneously increase the localization of viral protein into the mitochondria.

The mitochondrial stress-induced MDV is intimately involved in cross-talk with ER. Interestingly, HIV RNA also localizes to host mitochondria and induces mitochondrial dysfunction. The exact mechanism of how either mitochondria-localized corona or HIV viral RNA causes mitochondrial dysfunction is unknown. Multiple mechanisms are likely involved. To date, several questions remain unanswered, including the mechanism for viral RNA importation inside the host mitochondria, whether viral nonstructural protein interaction with Tomm70, a mitochondrial import receptor is involved in RNA importation into the mitochondria, whether noncoding RNAs encoded by coronavirus inhibit mitochondrial transcription, protein translation, or tRNA processing, whether there are cassettes of the coronavirus RNA genome that can be read using the mitochondrial amino acid translation code, and whether the formation of ER-derived DMV and mitochondria-derived MDV resemble one another mechanistically and whether mitochondrial MDV indeed serves as a reservoir for CoV-2 replication.

Mitochondria function as a platform for innate immune signaling. Notably, the host responses against viral infections depend on mitochondrial functions. Indeed, mtDNA itself acts as a danger-associated molecular pattern (DAMP). The mitochondrial outer membrane functions as a platform for signaling molecules. With age, mtDNA content and mitochondrial function decline. Mutations in mtDNA are also reported to accumulate with age, resulting in mitochondrial dysfunction, inflammation, and alterations in immune response.

ORF3a is a 275-amino acid protein with one amino acid less

in CoV-1. The CoV-1 and CoV-2 ORF3a sequences show 71.64% identity with each other, with 86% similarities. We observed that 174-GTTSPIS-180 aa motif shows an epitope tag, as it formed exposed regions that allow the protein to open up. The relative surface accessibility for these exposed regions is also unique compared with the CoV-2. ORF3a includes a 20-base sequence that targets USP30, a mitochondrial ubiquitin-specific peptidase 30, a subunit of ubiquitin ligase complex (FBXO21). The 20 nucleotides present in ORF3a of SARS-CoV-2 target the sequence AAAGATAGAGAAAAGGGGCT found in USP30 transcripts. USP30 is a mitochondrial deubiquitinase involved in mitochondria homeostasis and controls mitophagy. Thus, it is hypothesized that SARS-CoV-2 might affect mitochondria function by altering ubiquitination and contribute to the suppression of immunity in COVID-19 patients.

A significant number of mitochondrial proteins have been found interacting with the viral proteins. These include Nsp8 interaction with mitochondrial MRPS2, MRPS5, MRPS25, and MRPS27 ribosomal proteins, ORF9c interaction with mitochondrial NDUFAF1 and NDUFB9, and Nsp7 interaction with mitochondrial NDUFAF2. Notably, both NDUFAF1 and -2 are critical players involved in the assembly of complex I. NDUFB9 is an essential subunit of complex I comprised of more than 40 subunits. Furthermore, viral M protein was described to interact with ATP1B1, ATP6V1A, ACADM, AASS, PMPCB, PITRM1, COQ8B, and PMPCA. These proteins are part of the critical metabolic pathways carrying mitochondrial metabolism. Approximately 55 genes were mitochondrial, and approximately 105 genes modulated cellular function in response to bioenergetics function.

Previously, studies have also reported interaction of Cov-1 Nsp2 interaction with prohibiting protein PHB and Nsp10 interaction with mtDNA-encoded COX II (complex IV) and NADH4L (complex I). The study also identified NDUFA10 as one of the master regulators of CoV-2 pathology. Unfortunately, the biological significance of such interaction is lacking. CoV-2 interactions with Tomm70 have been found. This mitochondrial import receptor plays a critical role in transporting proteins into mitochondria and, more importantly, in modulating antiviral cellular defense pathways. These protein-protein interactions may give coronavirus a local advantage in manipulating mitochondria to suppress anti-viral response and host immunity.

To explore the host targeting by the viral proteins, we inspected the subcellular localization of ORFs and observed that ORF7a and ORF8 contain NH2-terminal signal peptide anchors. Although from the predictions they are shown to harbor signal peptides, the attachment of proteins to the extracellular membrane is prenylated. It appears that the combinatorial occurrence of domains and motif is associated with the proteins. For example, ORF3a shows motif exchange and similarity with LWLC associated with viral expression. Likewise, ORF7a is related to a motif ADNK, which might have evolved to resemble host protein motifs associated with the respiratory mechanisms in the host cells. These motifs indicate that they are functionally mimicking the binding sites of the proteins in carrying out the interactions. Although the sequence similarity and motifs are strain/sample specific, the superfamily domains these proteins are associated with do not show any particular links to human homologs.

Compared with women, SARS-CoV-2 infection results in a high rate of mortality in older men. The underlying cause of the high frequency is unknown. Both androgen and estrogen are known to regulate mitochondrial function. TMPRSS2 is induced by androgen and regulated by androgen receptor. It is hypothesized that a high prevalence of mortality in men may be due to androgenic induction of TMPRSS2. Furthermore, androgen receptors as well as estrogen receptors localize the mitochondrial compartment and regulate mitochondrial function. It is hypothesized that the hormonal regulation of mitochondrial targets and functions differs between men and women, which may contribute to a difference in susceptibility. Other hallmarks of aging, such as changes in epigenetic patterns, exhibiting different expressions in older men than age-matched women, may underlie factors involved in sex-specific mortality.

Differences in mitochondrial genetic makeup contribute to functional differences in the mitochondrial function in tissues, organs, and organisms as a whole. Thus, mitochondrial variants have different metabolic capacities, resulting in differences in anti-viral and inflammatory responses. Mitochondrial function declines with age and consistently reduces the level of mtDNA. Studies at the level of mtDNA in plasma of COVID-19-affected people and their ages may help in understanding whether the mtDNA-dependent systemic inflammation is deranged in aged people or whether individuals (or populations), albeit at young ages, may be endowed with mtDNA variants that are abnormally proinflammatory/and prone to being released.

Mitochondrial dysfunction is one of the hallmarks of aging that contributes significantly to the physiology of aging as well as the pathophysiology of age-related disorders. In most of the COVID-19 patients, systemic hyper inflammation leads to severe and often lethal outcomes. Aging is also characterized by systemic inflammation described as inflammaging and immunosenescence, an impairment of acquired immunity. It is conceivable that the decline in these critical players of aging contributes to the high rate due to CoV-2 infection.

Mitochondrial dysfunction induces senescence. Senescent cells are accumulated during aging and acquire the senescence-associated secretory phenotype (SASP), which contributes to inflammaging. Senescence impairs macrophages, which protect against infection. Macrophages are critical factors in protecting the lung after infection by SARSCoV-2. It is likely that older individual senescent macrophages are less active in protecting inflammation induced by COVID-19 and cause lethality. These observations support the notion that aging-associated inflammaging and immune senescence compromise robust responses as in young individuals to protect against the SAR-CoV-2 high rate of mortality.

Notably, the clinical manifestations in the most severe patients show a cytokine storm in which interleukin-6 (IL-6) values turn out to be significantly high. The extent of inflammaging differs between aged men and woman. High levels of IL-6 are found in older men compared with age-matched women. High levels of IL-6 may result in negative outcomes of COVID-19 in older men affected by comorbidity.

Another mechanism involves mtDNA directly to trigger host immunity. In this mechanism, upon viral infection, mtDNA leaks out of the mitochondria, into the cytoplasm, and into the extracellular space and induces innate immunity and inflammation. This is an evolutionarily conserved signaling mechanism induced upon many types of viral infection. Interestingly, high levels of circulating mtDNA are reported in older individuals. Older individuals suffering from COVID-19 may release mtDNA, resulting in the onset of a detrimental inflammatory response.

Additional factors that may contribute to age-related mortality include differences in ACE2 expression between young and older individuals. Several studies suggest that ACE2 is downregulated in aging, which may contribute to the increased risk of vascular injury and cardiovascular disease affecting older men. ACE2 is also downregulated in patients with type 2 diabetic men with severe COVID-19 outcomes. In contrast, ACE2 is overexpressed in women compared with men.

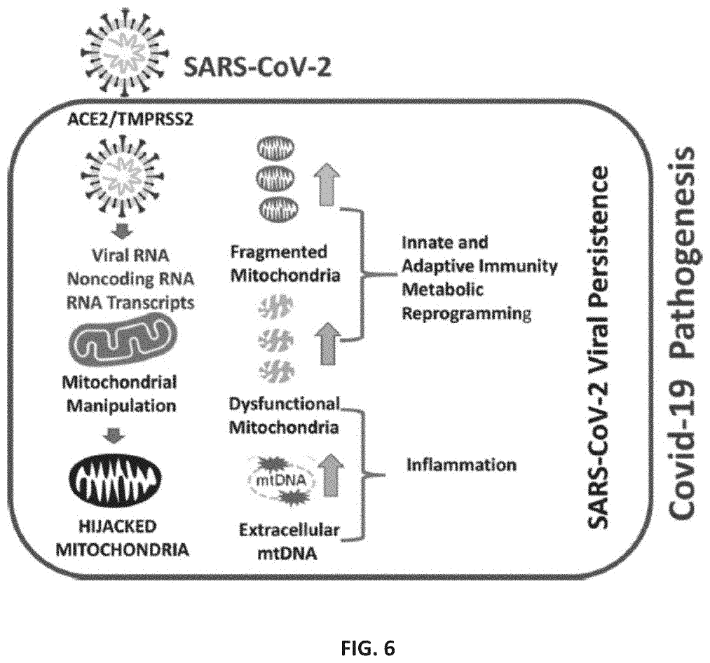

FIG. 6 is a schematic diagram summarizing the mechanisms involved in SARS-CoV-2 hijacking of host mitochondria. FIG. 6 includes a schematic showing the SARS-CoV-2 entry into the host cell utilizing angiotensin-converting enzyme carboxypeptidase 2 (ACE2), a polymorphic protein that regulates mitochondrial function. Upon entry into the cells, viral RNA and proteins localize to mitochondria. Post-infection noncoding RNA may also regulate host proteins (such as USP30) involved in mitochondrial dynamics. SARS-2-CoV-2 appears to hijack host mitochondria to suppress host immunity by regulating mitochondrial dynamics, mitochondrial function, and mtDNA release. Hijacking mitochondria may be one of the essential mechanisms leading to COVID-19.

Mitochondria serve as a signaling platform for mitochondrial anti-viral signal (MAVS). Mitochondria are involved in inflammation and both innate and adaptive immunity. Many single-strand positive RNA viruses similar to SARS-CoV-2 induce an inflammatory response that involves the mitochondrial biogenesis, mitochondrial fusion, fission, and mtDNA release to outside the cell. This phenomenon is described as neutrophil extracellular trap (NET)osis mechanisms (based on NET formation). The release of mtDNA is an ancestral cell stress mechanism that triggers local and systemic trigger of inflammation. Upon cell stress or damage, mtDNA leaks out into the cytoplasm and then outside, where it triggers inflammation and anti-viral response, which involves cytoplasmic AIM2 and/or endosomal/extracellular TLR9. Levels of mtDNA increase with the severity of damage and correlate with the onset of multiorgan failure in patients affected by multiple systemic conditions observed with acute respiratory distress.

It is likely that drugs that modulate mitochondrial function and inhibit inflammation may help treat patients with COVID-19. In particular, the observations summarized in FIG. 6 suggest that drugs that selectively restore mitochondrial function and promote mitochondrial biogenesis may serve as anti-inflammatory agents to prevent or treat COVID-19.

### Example 2: Mitochondrial Metabolic Manipulation by SARS-CoV-2 in Peripheral Blood Mononuclear Cells of Patients with COVID-19

In example 1 it was proposed that SARS-CoV-2 can hijack host mitochondrial function and manipulate metabolic pathways for their own advantage. In the present study, functional mitochondrial changes in live peripheral blood mononuclear cells (PBMCs) from patients with COVID-19 were investigated. The purpose was to decipher the pathways of substrate utilization in these cells and corresponding changes in the inflammatory pathways. Mitochondrial dysfunction, metabolic alterations are demonstrated with an increase in glycolysis, and high levels of mitokine in PBMCs from patients with COVID-19. It was found that levels of fibroblast growth factor 21 mitokine correlate with COVID-19 disease severity and mortality. These data suggest that patients with COVID-19 have a compromised mitochondrial function and an energy deficit that is compensated by a metabolic switch to glycolysis. This metabolic manipulation by SARS-CoV-2 triggers an enhanced inflammatory response that contributes to the severity of symptoms in COVID-19.

Mitochondrial dysfunction and associated oxidative stress drive the production of proinflammatory cytokines that, in turn, play an important role in the immune response. Fibroblast growth factor 21 (FGF-21), also known as a mitokine, is a hormone that is expressed in several metabolically active organs and regulates many important metabolic pathways. FGF-21 was proposed as a biomarker of mitochondrial dysfunction, and many diseases are characterized with alterations of mitokine secretion.

To explore the manipulation of mitochondrial function by SARS-CoV-2 and the induction of metabolic adaptions with corresponding cytokine changes in patients with COVID-19, fibroblast growth factor 21 (FGF-21) and interleukin-6 (IL-6) were measured in plasma of the patients with COVID-19, patients with chest infection, and healthy controls (HC).

### Materials and Methods

Patients with RT-PCR-positive COVID, clinically suspected COVID, and chest infections were recruited to the immunometabolism in sepsis, inflammation, and liver failure syndromes (I-MET) cohort observational study within 48 h of admission to an intensive care unit or a specialist ward. Healthy volunteers were also recruited as pathological controls.

The patients were studied in three groups: group 1 included healthy controls (n=9) who had no history of any chronic disease were recruited; group 2 included patients with PCR-positive COVID-19 or patients with strong clinical suspicion on radiological imaging (n=7); and group 3 included patients with chest infection who were PCR-negative for COVID-19 (n=7).

The 14 participants with PCR-positive COVID-19 and chest infection were aged 54-80 yr (mean=66), and the female:male ratio was 5:9. The average body mass index (kg/m²) in the group was 29±6.5 kg/m². Subjects were of mixed ethnic background (43% white British, 21% Caribbean, 21% Black ethnic group, and 15% others). Out of the 14 subjects, seven (50%) had type 2 diabetes mellitus (T2DM) and five (36%) were diagnosed with hypertension. Of the patients who were admitted in the intensive therapy unit (ITU; n=9), 67% were diagnosed with T2DM. Among the subjects with COVID-19, 71% had T2DM and 29% were diagnosed with hypertension.

Glycated hemoglobin (HbA1c) was the only parameter that was significantly higher in patients with COVID-19 as compared with chest infection. The ethnicities of all patients were mixed. All the subjects with COVID-19 who died (n=3, 43%) were diagnosed with T2DM and had an average National Early Warning Score (NEWS) 2 of 8.7.

Blood samples were obtained from the study participants. PBMCs were separated and plated on XFp eight-well polystyrene plates designed for the Seahorse XFp analyzer (Agilent Technologies, Santa Clara, Calif.) within 2 h. The Seahorse XFp analyzer (Agilent Technologies) was used to measure basal, ATP-linked, and maximal oxygen consumption rate (OCR); reserve capacity; and extracellular acidification rate (ECAR).

OCR, a measurement of mitochondrial respiration, and ECAR, which correlates to the number of protons released from the cell with potential contribution from glycolysis and the Krebs cycle, were measured in the presence of specific mitochondrial activators and inhibitors. Oligomycin (ATP synthase blocker) was used to measure ATP turnover and to determine proton leak; the mitochondrial uncoupler carbonyl cyanide 4-[trifluoromethoxy] phenylhydrazone (FCCP) was used to measure maximum respiratory function (maximal OCR).

Reserve capacity was calculated as maximal OCR minus the basal respiration. At the end of the experiments, rotenone (inhibitor of complex I) and antimycin A (a blocker of complex III) were injected to completely shut the mitochondrial respiration down, to confirm that any changes observed in respiration were mitochondrial.

For a measurement of basal respiration, three measurements were taken before injecting ATP synthase inhibitor oligomycin at 0.75 μM (final concentration). FCCP was then injected at 0.75 μM. Finally, a mixture of rotenone and antimycin A (1 μM) was injected. Mitochondrial basal respiration, proton leak, reserve capacity, and maximal respiration were measured after correcting for nonmitochondrial respiration.

OCR and ECAR rates were normalized to cell count for PBMCs. The XF substrate oxidation stress tests combine the substrate pathway-specific inhibitors: etomoxir (Eto) for LCFAs through inhibition of carnitine palmitoyltransferase 1a (CPT1a), UK5099 for glucose and/or pyruvate through inhibition of the mitochondrial pyruvate carrier (MPC), and bis-2-(5-phenylacetamido-1,3,4-thiadiazol-2-yl) ethyl sulfide (BPTES) for inhibition of glutamine through glutaminase 1 (GLS-1). For the substrate oxidation stress tests, etomoxir was added at 4 μM, UK5009 at 2 μM, and BPTES at 3 μM final concentrations.

Human FGF-21 SimpleStep ELISA Kit (Abcam, Cambridge, UK) was used to measure FGF-21 in plasma samples. Human IL-6 DuoSet ELISA (R&D systems, MN) was used to measure IL-6 in all samples.

### Results

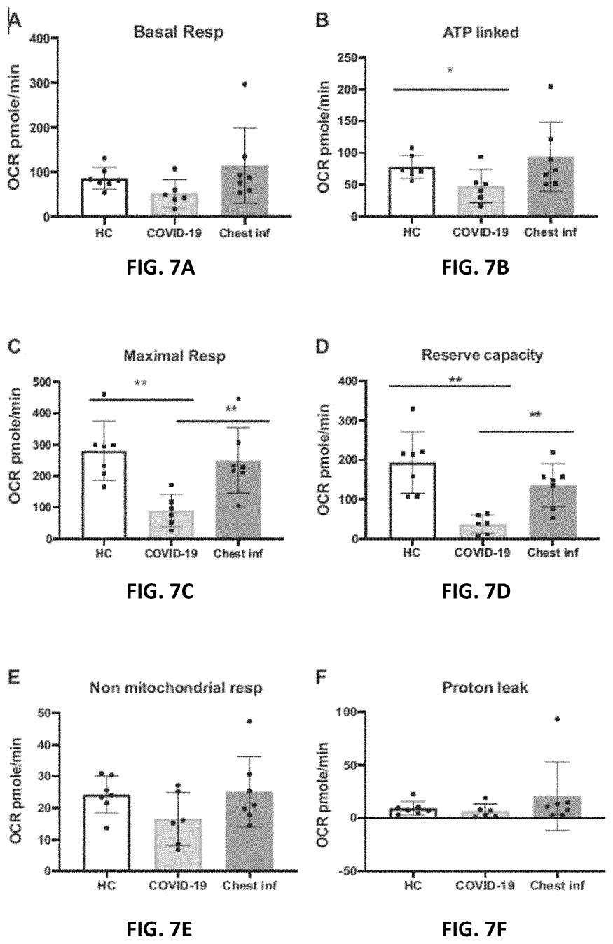

The results are shown in the graphs of FIGS. 7A-7F. Basal respiration was less, although not statistically significant, but there was a trend for significance (P=0.05) in patients positive for COVID-19 compared with in HC (FIG. 7A). ATP-linked respiration was also significantly reduced in patients positive for COVID-19 compared with HC (P=0.02; FIG. 7B), and reserve capacity was significantly reduced in patients positive for COVID-19 compared with HC (P=0.001) and patients with chest infection (P=0.004; FIG. 7D). Maximal respiration was also reduced in COVID-19-positive patients compared with in HC (P=0.002) and patients with chest infection (P=0.004; FIG. 7C). Proton leak (FIG. 7E) and nonmitochondrial respiration (FIG. 7F) were similar in the three groups. Significantly reduced ATP-linked respiration, reserve capacity, and maximal respiration are indicative of a compromised mitochondrial functional response to SARS-CoV-2 infection.

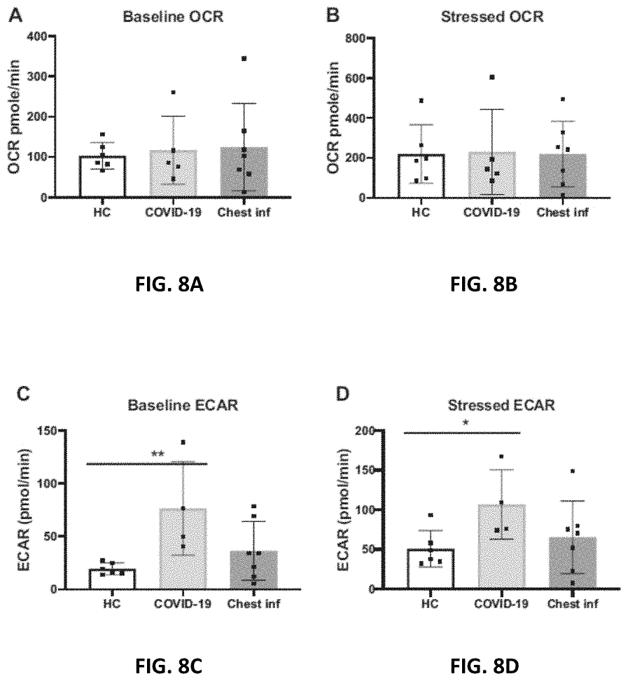

Increased glycolysis was observed in patients with COVID-19. The results are shown in the graphs of FIGS. 8A-8C. OCR as a measure of mitochondrial respiration and ECAR as a measure of glycolysis were determined under baseline and stressed conditions, to reveal the baseline and stressed phenotype of PBMCs in patients with COVID-19. There was a lack of significant difference in basal and stressed OCR among COVID-19-positive individuals, patients with chest infection, and HC (FIGS. 8A and 8B). Interestingly, the basal ECAR was significantly higher in patients with COVID-19 as compared with in HC (P=0.009; FIG. 8C). Stressed ECAR was also higher as compared with HC (P=0.03; FIG. 8D). We conclude that the high basal and stress ECAR suggest that the PBMCs of patients with COVID-19 depend on glycolysis for energy.

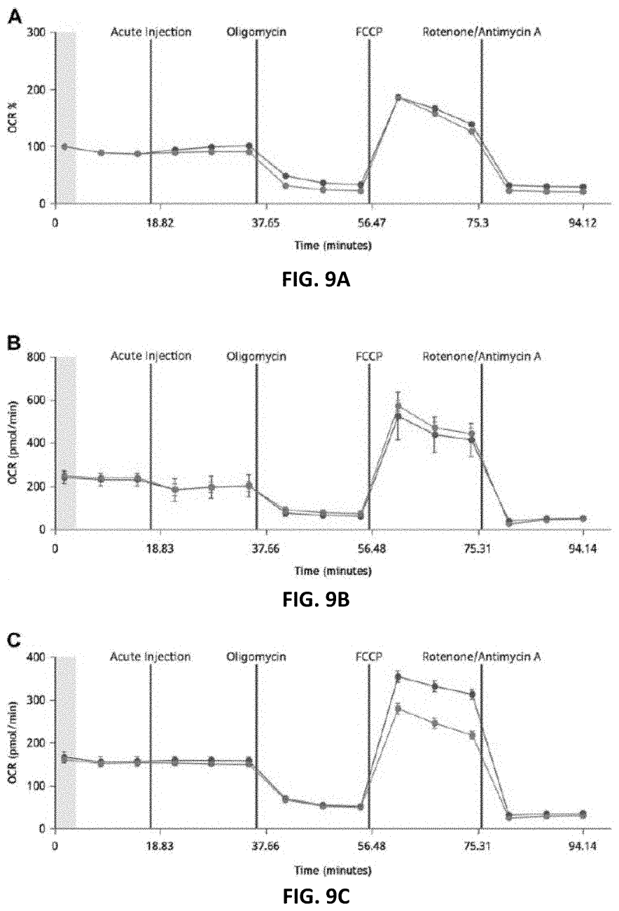

Glucose was found to serve as the main substrate used by mitochondria in SARS-CoV-2. The results are shown in FIGS. 9A-9C. The dependency, capacity, and flexibility of cells to oxidize three mitochondrial fuels, namely, glucose (pyruvate), glutamine (glutamate), and long-chain fatty acids (LCFAs), were determined. Fuel flexibility, which is a difference between fuel capacity and dependency, is the ability of cells to increase oxidation of a particular fuel to compensate for inhibition of alternative fuel pathway. Two assays were performed and both showed 100% flexibility for glucose utilization in COVID-19 samples. To reveal the critical substrate dependence/reliance, we performed substrate utilization tests (n=2) for glucose, glutamine, and LCFAs individually.

Bis-2-(5-phenylacetamido-1,3,4-thiadiazol-2-yl) ethyl sulfide (BPTES) was used for inhibition of glutamine through glutaminase 1, and there was no difference in basal respiration (before blocking=73±0.95 vs. after blocking=78±5.6) and maximal respiration (before blocking=198±12 vs. after blocking=192±12) for glutamine (FIG. 9A). Similarly, etomoxir (Eto) was used for inhibition of LCFAs through inhibition of carnitine palmitoyltransferase 1a (CPT1a). There was no difference in basal respiration (before blocking=134±6 vs. after blocking=128±2) and maximal respiration (before blocking=271±25 vs. after blocking=255±16) for LCFAs (FIG. 9B). UK5099 was used for inhibition of glucose or pyruvate through inhibition of the mitochondrial pyruvate carrier (MPC). In patients with COVID-19, basal respiration (before blocking=126±18 vs. after blocking=128±5) was not different but maximal respiration was reduced after blocking the glucose pathway (before blocking=323±22 vs. after blocking=254±24; FIG. 9C). In HC, there was no difference in basal respiration (before blocking=80±33 vs. after blocking=52±28) and maximal respiration (before blocking=37±25 vs. after blocking=34±5) after blocking the glucose pathway. In patients with chest infection, there was no difference in basal respiration (before blocking=200±35 vs. after blocking=195±33) and maximal respiration (before blocking=589±16 vs. after blocking=558±19) after blocking the glucose pathway. These studies demonstrating a decrease in maximal respiration in patients with COVID-19 after blocking the glucose pathway suggest dependency of PBMCs on glucose.

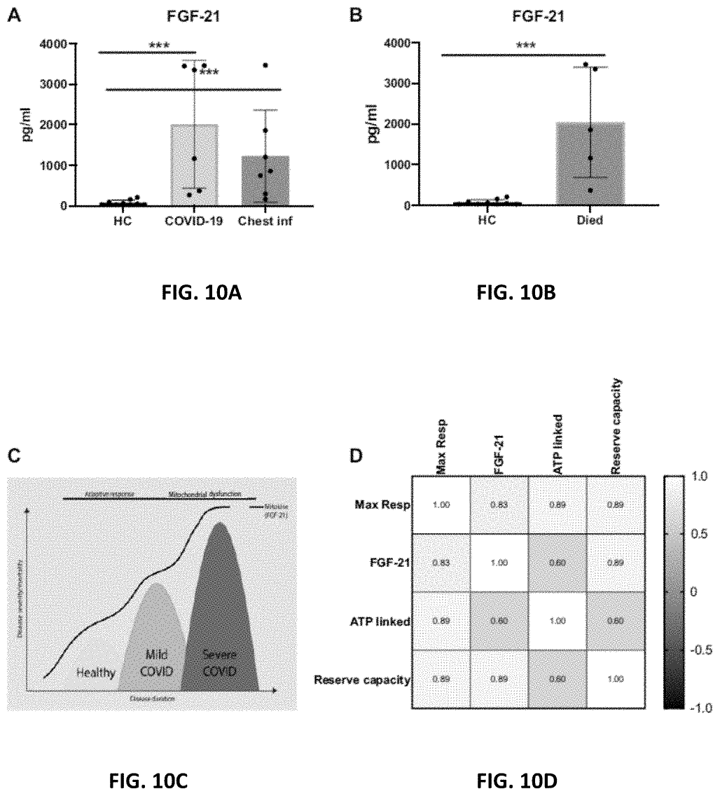

Increased fibroblast growth factor 21 levels were observed in plasma of patients with COVID-19. The results are shown in FIGS. 10A-10D. Plasma levels of fibroblast growth factor 21 (FGF-21) were measured by ELISA in HC (n=9), patients with COVID-19 (n=7), and patients with chest infection (n=7). FGF-21 levels were significantly higher in patients with COVID-19 (P=0.0004) and chest infection (P=0.0002) as compared with in HC (FIG. 10A). FGF-2 levels were also significantly higher in patients with COVID-19 who died as compared with in HC (P=0.001; FIG. 10B). The patients admitted to the intensive therapy unit (ITU) with severe symptoms of COVID-19 had significantly higher values of FGF-21 as compared with HC (P<0.0001; FIG. 10C). There is also correlation between FGF-21 levels and mitochondrial reserve capacity (P=0.03; FIG. 10D). The NEWS score correlation with FGF-21 for patients with COVID-19 who died was r=0.85. These studies suggest a correlation between increased FGF-21 mitokine and COVID-19.

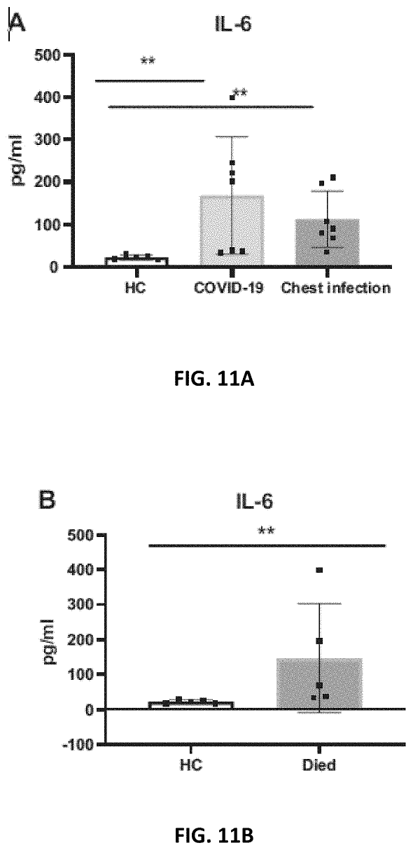

High human interleukin-6 was observed in plasma of patients with COVID-19 by ELISA. The results are shown in the graphs of FIGS. 11A-11B. Human IL-6 DuoSet ELISA (R&D systems, MN) was used to measure IL-6 levels in HC (n=5), patients with COVID-19 (n=7), and patients with chest infection (n=7). IL-6 levels were significantly higher in patients with COVID-19 (168±138, P=0.002) and chest infections (112±66, P=0.002) as compared with in HC (22±5; FIG. 11A). There was a trend of higher values of IL-6 in patients admitted to the ITU (165±120) with severe symptoms of COVID-19 as compared with those in wards (96±76); however, this was not significant. IL-6 levels were also significantly higher in patients who died due to COVID-19 (147±156) as compared with in HC (22±5, P=0.007; FIG. 11B). These results indicate that high levels of IL-6 are linked to COVID-19 mortality.

### Discussion

The current study was undertaken to determine the role of mitochondrial function and associated metabolic changes that may be involved in the inflammatory response during COVID-19 pathogenesis. We show mitochondrial dysfunction in live PBMCs of patients with COVID-19 and demonstrate an increased rate of glycolysis and utilization of glucose as the main substrate for energy production. There was a corresponding increase in FGF-21 mitokine and IL-6 in patients with severe symptoms and higher mortality rate in COVID-19 disease. FGF-21 levels showed significant correlation with mitochondrial functional reserve capacity.

Our study indicates that SARS-CoV-2-infected cells have compromised mitochondrial respiration but can use glucose for energy and cell survival. Together our studies suggest that the mitochondrial manipulation by SARS-CoV-2 may provide an advantage for viral replication and increase cytokine production.

In example 1, the potential hijacking of host mitochondria by SARS-CoV-2 as a mechanism underlying COVID-19 pathogenesis. Mitochondria function as a platform for innate immune signaling. Notably, the host responses against viral infections depend on mitochondrial functions. Mitochondrial DNA itself acts as a danger-associated molecular pattern (DAMP).

Previously, it has been shown that SARS-CoV-1 virus upon infection affects mitochondrial functions, influences its intracellular survival, or evades host immunity. SARS-CoV-1 ORF-9b localizes to host mitochondria, which suppresses innate immunity by manipulating mitochondrial function and the mitochondrial antiviral-signaling protein (MAVS)/tumor necrosis factor receptor-associated factor (TRAF 3 and 6) signaling pathway to host innate immunity. Mitochondria-localized CoV-1 ORFs were compared with ORFs encoded by the CoV-2 genome. Except for ORF3b, SARS-CoV-2 encodes an amino acid sequence similar to SARS-CoV-1 ORFs (ORF7a, 8a, and 9b) that are localized to host mitochondria.

Cells contain two important energy-producing pathways: mitochondrial respiration and glycolysis. Using the Seahorse XFp analyzer, we simultaneously measured both of these pathways in live cells in a multi-well plate, interrogating key cellular functions. Our data show that peripheral cells from patients with PCR-positive patients exhibit reduced maximal respiration and reserve capacity, indicating compromised mitochondrial respiration or mitochondrial dysfunction, as compared with HC. These cells, however, have high basal and stress glycolysis, indicating the ability to use glucose for energy.

We also performed the Mito Fuel Flex Test which showed that these cells have 100% fuel flexibility for glucose, which means that these cells can increase oxidation of glucose to compensate for inhibition of the alternative fuel pathway. To investigate this in detail, we performed substrate utilization tests for glutamine, long-chain fatty acids, and glucose in PBMCs of patients with COVID-19. There was no difference in basal and maximal respiration on blocking glutamine and long-chain fatty acid pathways; however, maximal respiration was reduced when the glucose pathway was blocked in cells from patients with COVID-19 as compared with from HC. This indicates dependence of SARS-CoV-2-infected cells on glucose for energy production and survival.

In our clinical cohort of subjects with COVID-19, 70% had T2DM, and of those admitted in the ITU with more severe symptoms, 80% had T2DM. All the patients with COVID-19 who died were diagnosed with diabetes. This indicates that high glucose level in T2DM favors SARS-CoV-2 replication and is associated with worse prognosis in these patients.

Recently, a study of influenza A virus (IAV) infection shows activation of similar metabolic changes resulting in a cytokine storm. Authors also show that higher levels of proinflammatory cytokines correlate with higher levels of blood glucose in patients infected with IAV. Studies have highlighted the high mortality rate in patients with diabetes due to COVID-19. Consistent with our study in patients with COVID-19, it has been determined that glycolysis is necessary for SARS-CoV-2 replication and monocyte response. It is known that mitochondrial stress induces the production of stress response molecules known as mitokines.

Many diseases are characterized by progressive mitochondrial dysfunction with alterations of mitokine secretion. It is still controversial whether altered levels of mitokines are beneficial or detrimental in humans. The function of FGF-21 mitokine is complicated due to its different sites of production and actions. Studies have previously shown high serum levels of FGF-21 in patients with metabolic disorders due to mitochondrial dysfunction. Circulating levels of FGF-21 are also elevated in type 2 diabetes (T2DM).

We also measured FGF-21, a mitokine, in patients with COVID-19 and HC. Mitochondrial dysfunction, oxidative stress, and inflammation can lead to a compensatory increase in FGF-21 synthesis and secretion. Our study demonstrates an increase in FGF-21 levels in patients with COVID-19 as compared with in HC. There was a trend of high levels of FGF-21 and an increase in COVID-19 severity among patients who died due to COVID-19. FGF-21 levels were the highest among the patients who died. The proinflammatory cytokines IL-6 was also significantly increased in patients with COVID-19 as compared with in HC (P=0.002). These findings strengthen the observation that mitochondrial dysfunction drives a systemic immune response in COVID-19 pathogenesis.

In summary, our study identifies the important metabolic alteration in live cells from patients with COVID-19. It paves the way to investigate whether restoring mitochondrial function and/or targeting glycolysis with new or existing drugs can potentially be used for treatment for patients with COVID-19.

### Example 3: Identification of Compounds from Emblica Extract

To identify compounds present in emblica, an emblica extract was prepared and analyzed by HPLC.

In this example, 40.9 grams of caplets containing E. officinalis extract (Himalaya Drug Company, Sugar Land, Tex. Lot 112000924; each caplet contains 250 mg fruit extract (45% tannins) and 350 mg powder stem (2% tannins)) was stirred with 250 ml methanol for 6 hours at room temperature. The resulting solution was filtered to remove insoluble material and the methanol layer was stripped using a Rotovap (fraction 1). The solid material resulting from the filtration step was stirred with an additional 250 ml of methanol for 4 hours at room temperature, filtered through to remove insoluble material and a filter funnel and the methanol layer was stripped using a Rotovap (fraction 2). Fractions 1 and 2 were combined to obtain 8.25 grams of a dark, hydroscopic solid.

450 mg of solid isolated as described above was dissolved in 2 ml of methanol. The resulting solution was sonicated until all contents were in solution and subject to preparative HPLC purification using a ACCQPrep instrument (Teledyne ISCO, Lincoln, Nebr.) with a Phenomenex Gemini® 5 μM NX-C18 110A 150×4.6 mm liquid chromatography column (Phenomenex, Torrance, Calif.). HPLC was carried out with a two solvent gradient as described in the Table 3 below.

TABLE 3
HPLC Gradient.

| Gradient | | | Flow rate |
|---|---|---|---|
| Time (min) | % Solvent A | % Solvent B | (ml/min) |
| 0 | 0 | 100 | 42.5 |
| 5 | 0 | 100 | 42.5 |
| 20 | 90 | 10 | 45.2 |
| 24 | 90 | 10 | 42.5 |
| 26 | 5 | 95 | 42.5 |
| 28 | 5 | 95 | 42.5 |
| 29 | 50 | 50 | 42.5 |
| 43 | 50 | 50 | 42.5 |

Solvent A-acetonitrile;
Solvent B-water
Water purified through a PureLab ® Ultra water purification system (ELGA LabWater, Woodridge, IL).

The collected fractions were stripped and fractions transferred into eleven separate vials. 1 mg of each fraction was taken and dissolved in 1 ml of methanol in a 1.5 ml vial. The resulting solution was sonicated until all contents were in solution and subject to analytical HPLC analysis using an Agilent 1100 Series instrument (Agilent Technologies, Santa Clara, Calif.) with a Phenomenex Gemini® 5 μM NX-C18 110A 150×4.6 mm liquid chromatography column (Phenomenex, Torrance, Calif.). HPLC was carried out with a two solvent gradient as described in the Table 4 below.

TABLE 4
HPLC Gradient.

| Gradient Time (min) | % Solvent A | % Solvent B | Flow rate (ml/min) |
|---|---|---|---|
| 0 | 0 | 100 | 1 |
| 5 | 0 | 100 | 1 |
| 20 | 97.5 | 2.5 | 1 |
| 24 | 90 | 10 | 1 |
| 26 | 5 | 95 | 1 |
| 28 | 5 | 95 | 1 |
| 29 | 100 | 0 | 1 |
| 43 | 100 | 0 | 1 |

Solvent A-acetonitrile with 0.1% trifluoroacetic acid (TFA)
Solvent B-water with 0.1% TFA
Water purified through a PureLab ® Ultra water purification system (ELGA LabWater, Woodridge, IL).

To identify specific compounds in the emblica extract the standards shown in Table 5 were used.

TABLE 5
Emblica Extract Compound Identification Standards.

| Common name | Cas # | Source | Retention Time (min) |
|---|---|---|---|
| Gallic Acid | 149-91-7 | Acros | 6.88 |
| | | Organics | 7.22 |
| vanillic acid | 121-34-6 | Alfa Aesar | 27.64 |
| chlorogenic acid | 327-97-9 | TCI America | 28.61 |
| caffenic acid | 331-39-5 | TCI America | 28.65 |
| Syringic acid | 530-57-4 | Acros | 28.86 |
| | | Organics | |
| tp coumaric acid | 501-98-4 | TCI America | 29.52 |
| quercetin | 849061-97-8 | Acros | 30.63 |
| | | Organics | |
| Vitamin C | 50-81-7 | Sigma | 2.36 |
| | | Aldrich | 3.3 |

Standards were prepared by dissolving 1 mg of the standard in 1 ml methanol, sonicating the solution, and adding the solution to a 2 ml vial. Samples were subject to analytical HPLC as described above and to validate purity. A combined standard solution was created by adding 100 ul of each standard to a separate 2 ml vial. The standards were used to identify components in the emblica extract.

The following compounds (Table 6) were identified in the emblica extract prepared and analyzed as described above.

TABLE 6
Compounds identified in emblica extract.

| | | Estimated amount |
|---|---|---|
| Common name | Cas # | in emblica tablets |
| Gallic Acid | 149-91-7 | 70% |
| vanillic acid | 121-34-6 | Less than 2% |
| chlorogenic acid | 327-97-9 | Less than 2% |
| caffenic acid | 331-39-5 | Less than 2% |
| Syringic acid | 530-57-4 | Less than 2% |
| tp coumaric acid | 501-98-4 | Less than 2% |
| quercetin | 849061-97-8 | Less than 2% |
| Vitamin C | 50-81-7 | 20% |

In addition, approximately 20 other compounds were present but have not yet been identified.

### Example 4: Prophetic Example Showing In Vivo Energy and Vitality Improving Effects of Application of Emblica Extract

Male and female subjects aged between 35 and 70 years showing aging in the form of visible eye wrinkles without further specific inclusion criteria will be evaluated for the study. The subjects will complete a baseline questionnaire of 10 closed questions with predefined options to be selected. The questionnaire will request information relating to baseline energy levels and vitality. Other baseline parameters will be measured for skin roughness (Ra, Rz) by DermaTOP (three-dimensional imaging of surface structure), skin hydration by Corneometer (Courage & Khazaka, Cologne, Germany) (electrical capacitance measurement), and skin elasticity by Cutometer (optical measurement of skin displacement during 300 mbar suction). The subjects will apply emblica extract (2-5 drops of emblica extract oil) or placebo twice daily topically to one side of the face for approximately 12 weeks. The study will be performed in a split-face design. Anti-wrinkling properties will be measured periorbitally in the region of crowfeet. Skin moisturizing effects will be evaluated on the bones of the cheeks. Effect of the emblica extract will be compared to the reference product or placebo.

Subjects will complete a final questionnaire at completion of the study. The final questionnaire will request information relating to baseline energy levels and vitality. Skin roughness (Ra, Rz), skin hydration, and skin elasticity will also be measured at the completion of the study. Baseline results will be compared to final results.

It is expected that administration of emblica extract will improve energy levels and vitality. It is also expected that administration of emblica extract will improve aging-associated parameters including skin roughness, skin hydration, skin elasticity, and additional qualitative parameters measured by the questionnaire.

### Example 5: Restoration of Mitochondrial DNA Depletion and Function and Suppression of Inflammation by Emblica Extract In Vivo

In these experiments, the shaved dorsal skin of 8-9 weeks old female C57BL/6 control and mtDNA-depleter mice (expressing D1135A-POLG1) were treated topically with 200 μl of 50 mg/ml emblica extract ointment (prepared as described in the Methods section) or a corresponding amount of the control ointment (lacking emblica extract) daily beginning 1 week prior to the start of dox administration (200 mg/kg diet only) and continuing daily applications for 16 weeks (112 days).

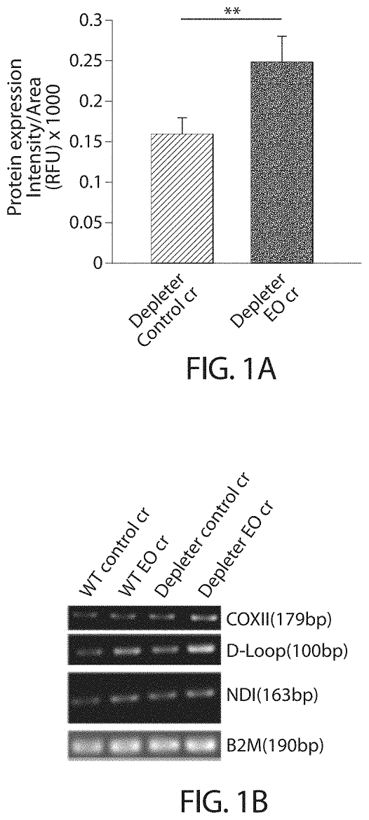

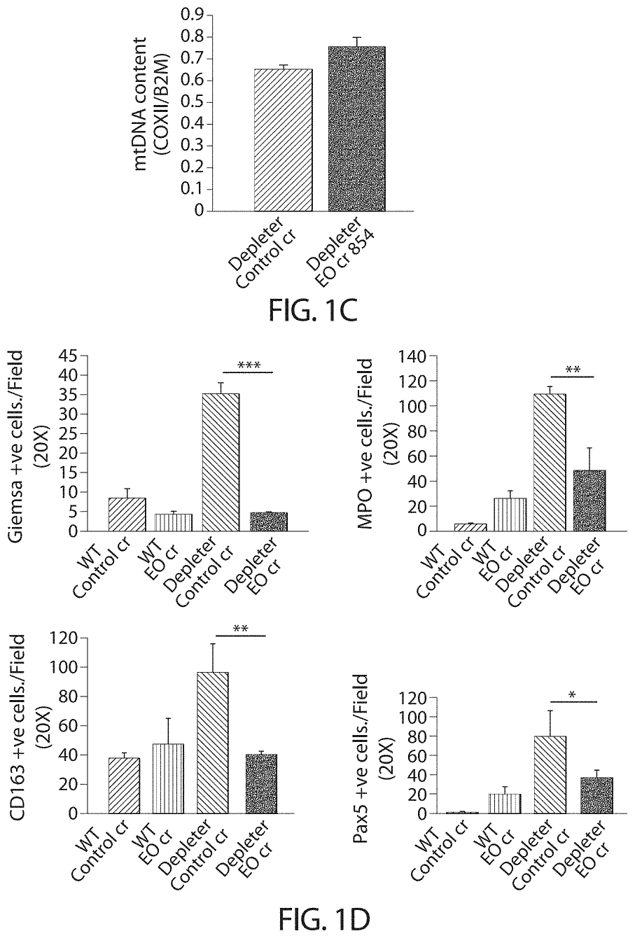

The effect of emblica extract treatment on reversal of mtDNA function was examined by staining paraffin embedded dorsal skin sections mtDNA-depleter mice treated with emblica extract ointment or control ointment with Oxphos complex IV antibody (COXII) (n=3). A statistically significant increase in COXII staining in mtDNA-depleter skin treated with emblica extract as compared to control treatment was observed (FIG. 1A). In addition, RT-PCR analysis of mtDNA encoded genes showed upregulation in mtDNA-depleter skin treated with emblica extract as compared to control treatment (FIG. 1B). Finally, an increase in mtDNA content was observed in skin samples from mtDNA-depleter mice treated with emblica extract as compared to control treatment (FIG. 1C).

As described herein, the skin of mtDNA depleter mice showed increase in dermal and peri-appendageal mixed inflammatory cells, including mast cells, neutrophils and lymphocytes. After treatment with emblica extract, the skin of mtDNA depleter mice showed a statistically significant decrease in dermal and peri-appendageal inflammatory cells (FIG. 1D) including mast cells (Giemsa+ve positive cells; p=3.56E-07), granulocytes (MPO +ve cells; P=0.002), macrophages and histiocytes (CD163 +ve cells; p=0.007), and B lymphocytes (Pax-5 +ve cells; p=0.042). Decreased expression of inflammatory genes in the skin samples of mtDNA-depleter mice treated with emblica extract as to mtDNA-depleter mice treated with control ointment was also observed (data not shown).

### Example 6: Restoration of Mitochondrial DNA Depletion, as Evidenced by Reversal of Wrinkled Skin and Loss of Hair by Fucus Extract In Vivo

To investigate the ability of other agents to restore mitochondrial DNA and function, a fucus extract was examined. Reversal of mitochondrial DNA depletion is evidenced by reversal of wrinkled skin and loss of hair. However, reversal of mitochondrial DNA depletion has a therapeutic effect on other indications.

In these experiments, the shaved dorsal skin of 8-9 weeks old female C57BL/6 control and mtDNA-depleter mice (expressing D1135A-POLG1) were treated topically with 200 μl of 50 mg/ml fucus extract ointment (prepared as described in the Methods section) or a corresponding amount of the control ointment (lacking fucus extract) daily beginning 1 week prior to the start of dox administration (200 mg/kg diet and 2 mg/mL in 5% sucrose water) and continued for 51 days (n=4 for each group).

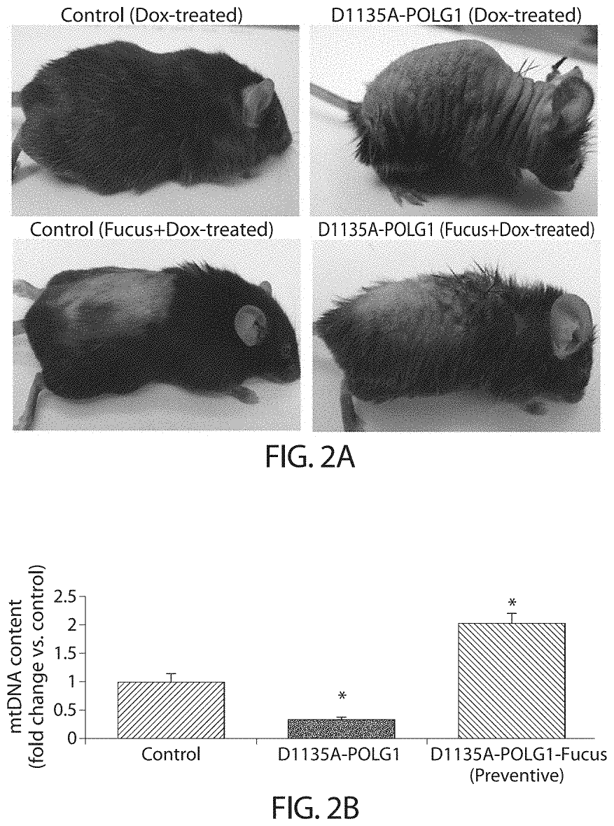

The results are shown in FIG. 2A. Consistent with previous results, dox administration to mtDNA-depleter mice resulted in significant hair loss and skin wrinkling phenotype. Administration of fucus extract partially reversed the hair loss and wrinkled skin phenotype as compared to mtDNA-depleter mice without fucus extract treatment (dox administration alone). Control mice showed no effects when treated with dox alone or dox in combination with fucus extract.

### Example 7: Restoration of Mitochondrial DNA Content by Fucus Extract In Vivo

The effect of fucus extract treatment on mitochondrial DNA content was also examined. Skin samples from the animals of Example 14 were collected and analyzed for mitochondrial DNA content and the results are shown in FIG. 2B. As shown in FIG. 2B, dox administration to mtDNA-depleter mice resulted in significant decrease in mitochondrial DNA content as compared to control mice. Administration of fucus extract fully restored mitochondrial DNA content. These results show that the beneficial effects of fucus extract on reversing the hair loss and wrinkled skin phenotype in mtDNA-depleter mice is correlated with preservation of mitochondrial DNA content.

### Example 8: Expression of Mitochondrial Biogenesis Regulatory Proteins In Vitro

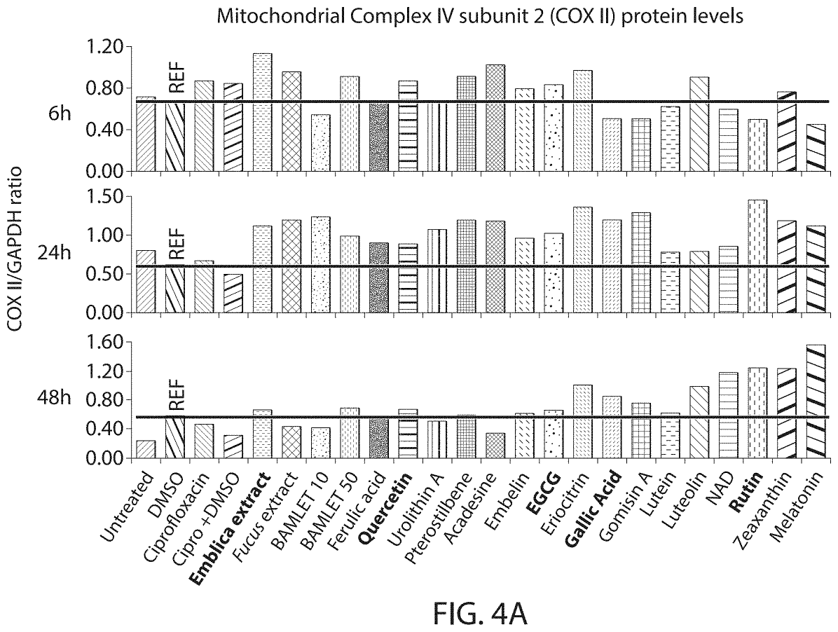

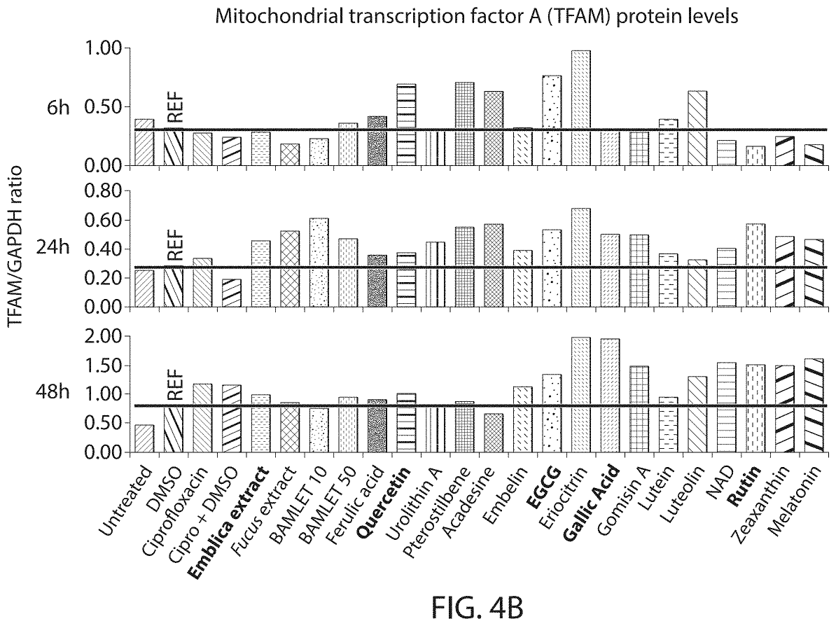

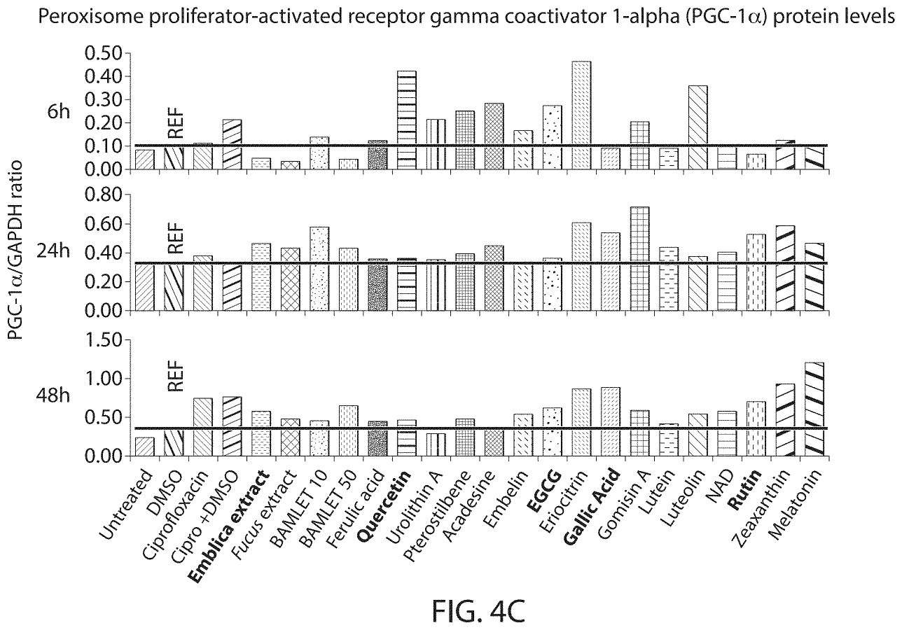

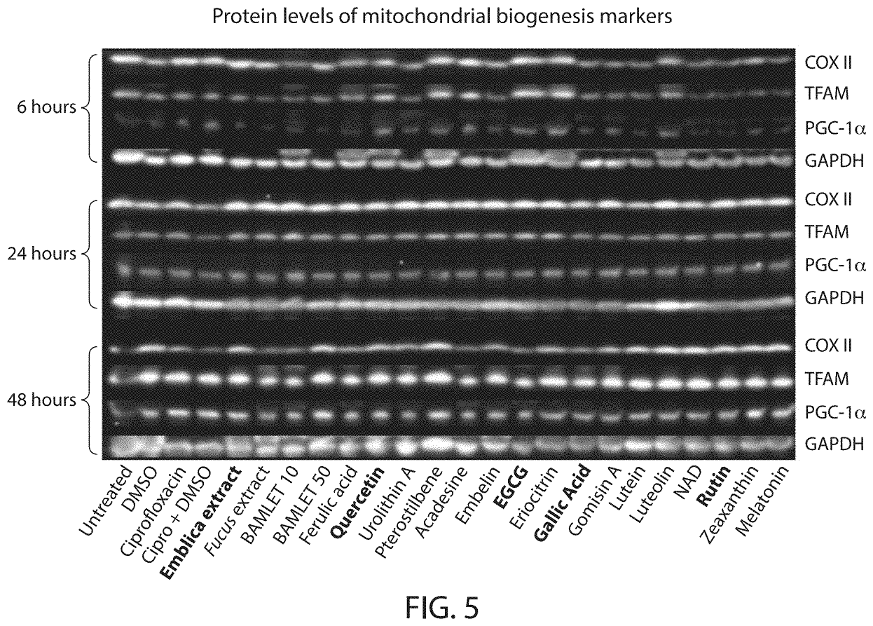

Emblica extract, components thereof, fucus extract, and related compounds were tested in vitro for induced expression of mitochondrial biogenesis regulatory proteins including mitochondrial complex IV subunit 2 (COXII), mitochondrial transcript factor A (TFAM), and peroxisome proliferator-activated receptor gamma coactivator 1-alpha (PGC-1a) at timepoints of 6 hours, 24 hours, and 48 hours post-administration. The results are presented in the graphs of FIGS. 4A-4C and the gel-electrophoresis image of FIG. 5.

Many of the tested compositions showed increased expression of the proteins as compared to a DMSO reference. Expression generally increased with increasing time.

### Example 9: Extended Expression of Mitochondrial Biogenesis Regulatory Proteins In Vitro

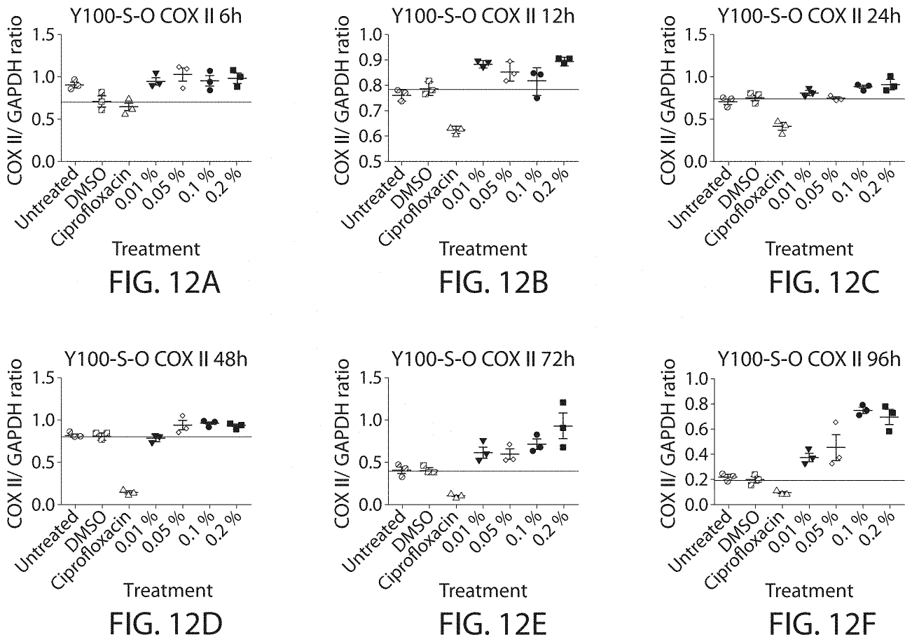

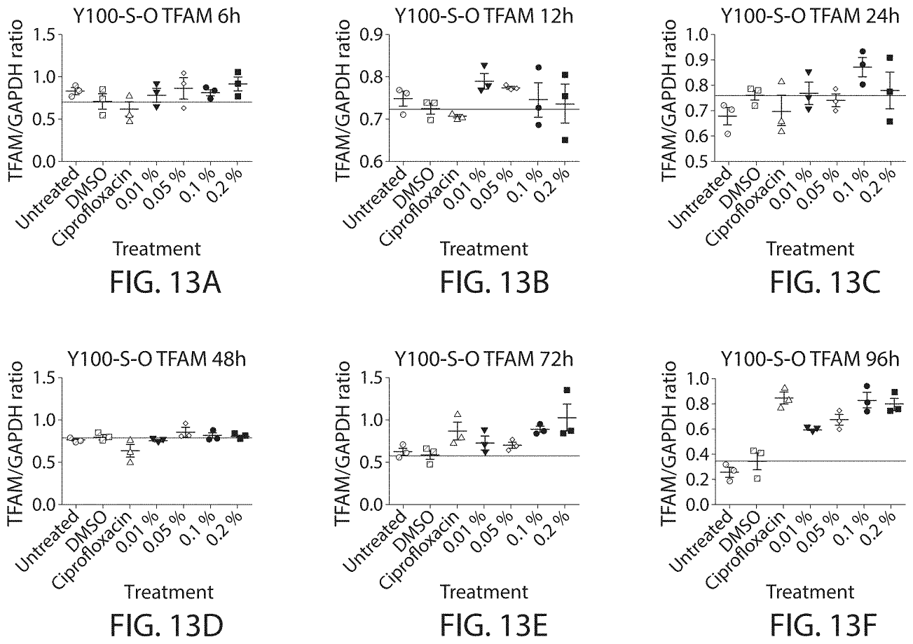

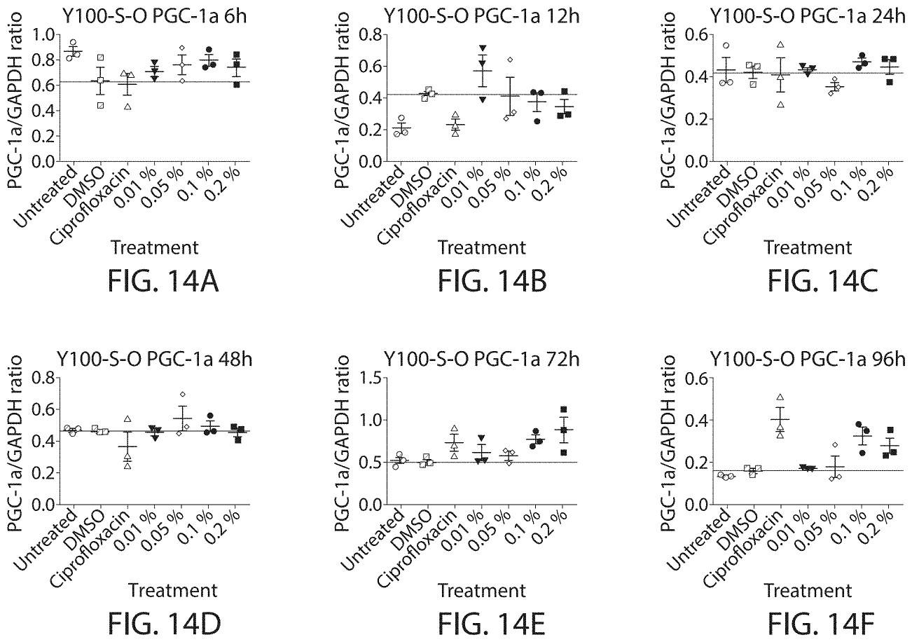

Emblica extract was tested in vitro for induced expression of mitochondrial biogenesis regulatory proteins including mitochondrial complex IV subunit 2 (COXII), mitochondrial transcript factor A (TFAM), and peroxisome proliferator-activated receptor gamma coactivator 1-alpha (PGC-1a) at timepoints of 6 hours, 12 hours, 24 hours, 48 hours, 72 hours, and 96 hours post-administration, generally as described in previous examples. Formulations of 0.01%, 0.05%, 0.1% and 0.2% emblica extract were tested. The results are presented in the graphs of FIGS. 12A-12F (COXII expression), 13A-13F (TFAM expression), and 14A-14F (PC1a expression). The general trend is that certain formulations induced greater expression after 96 hours than at earlier timepoints.

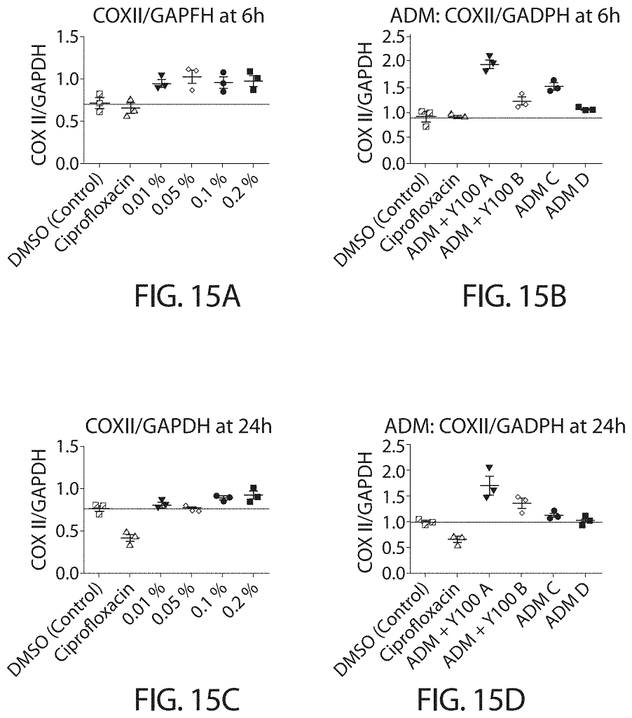

Emblica extract formulated with a nanoparticle delivery carrier was tested in vitro for induced expression of COXII and compared to a non-encapsulated composition at timepoints of 6 hours and 24 hours post-administration. The results are presented in the graphs of FIGS. 15A-15D. Specifically, FIG. 15A shows COXII expression at 6 hours after administration of a non-encapsulated composition. FIG. 15B shows COXII expression at 6 hours after administration of a nanoencapsulated composition. FIG. 15C shows COXII expression at 24 hours after administration of a non-encapsulated composition. FIG. 15D shows COXII expression at 24 hours after administration of a nanoencapsulated composition.

As shown in the data presented in FIGS. 15A-15D, all emblica extract formulations showed an increase in COXII expression and certain nanoencapsulated formulations showed a greater increase in COXII expression.

### Example 10: Expression of Mitochondrial Biogenesis Regulatory Proteins In Vitro

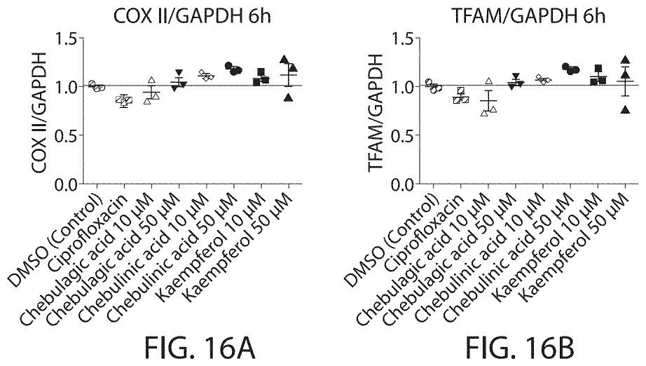

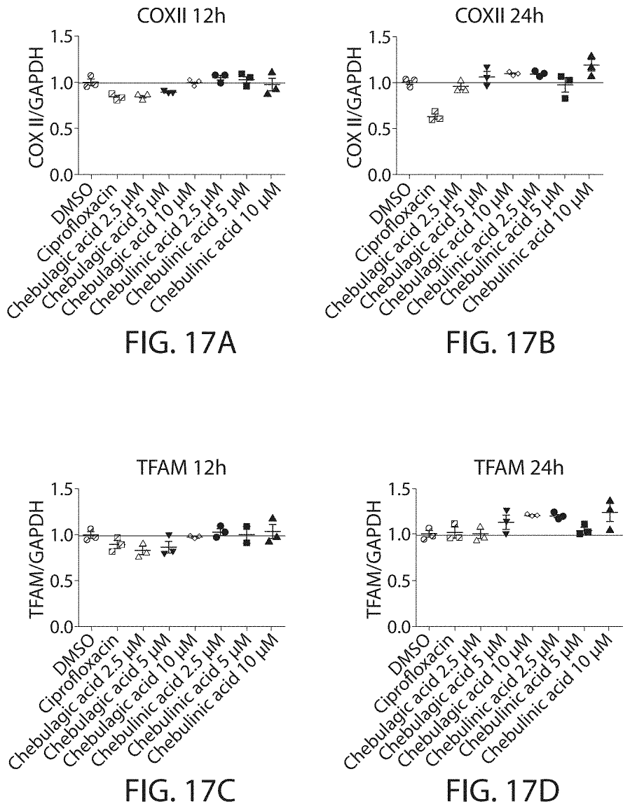

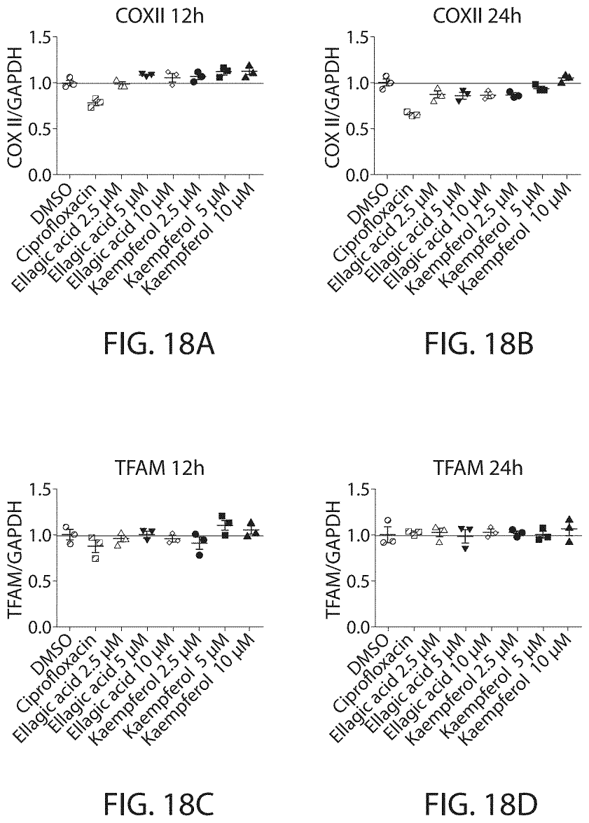

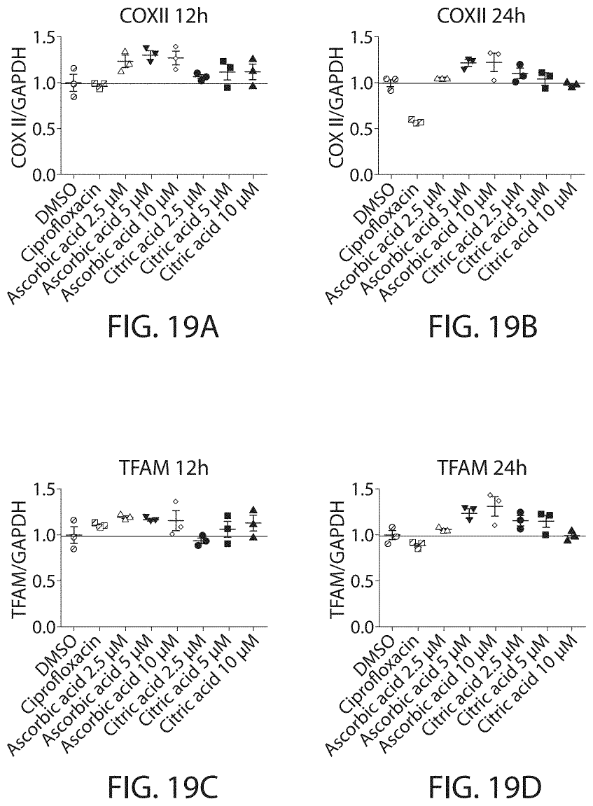

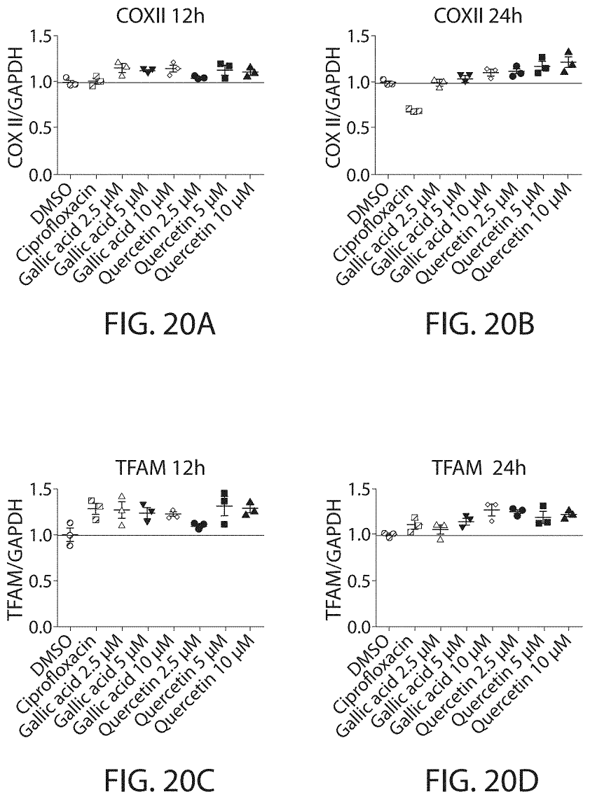

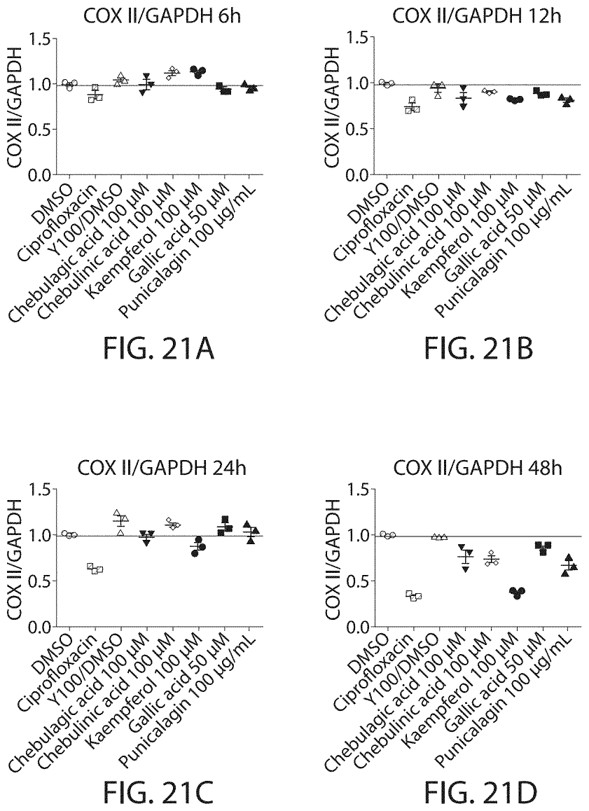

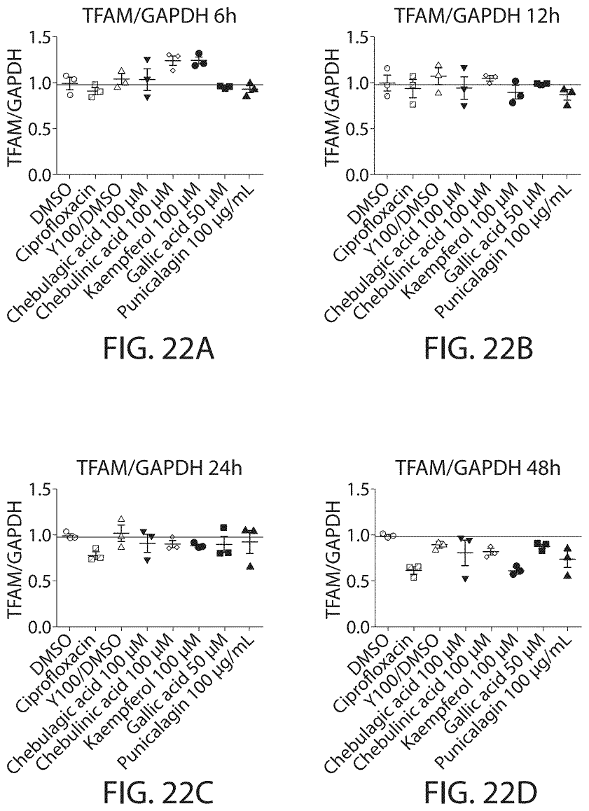

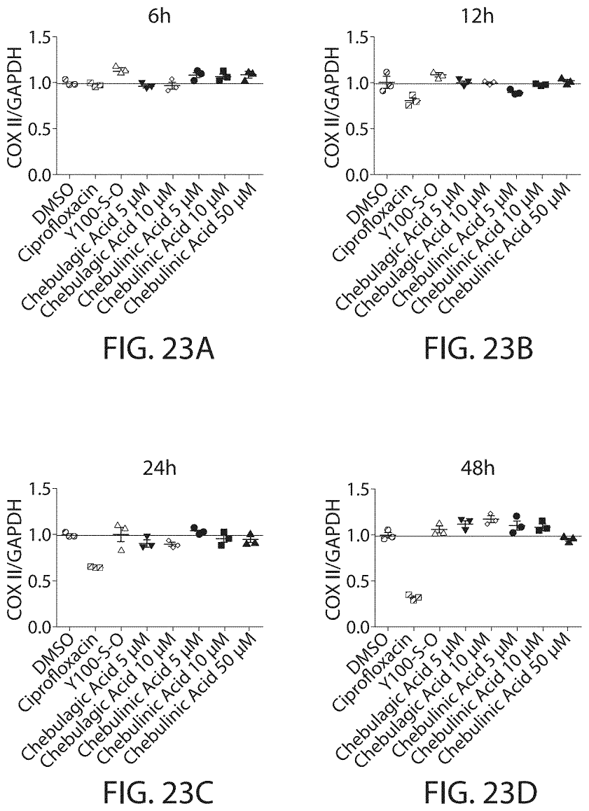

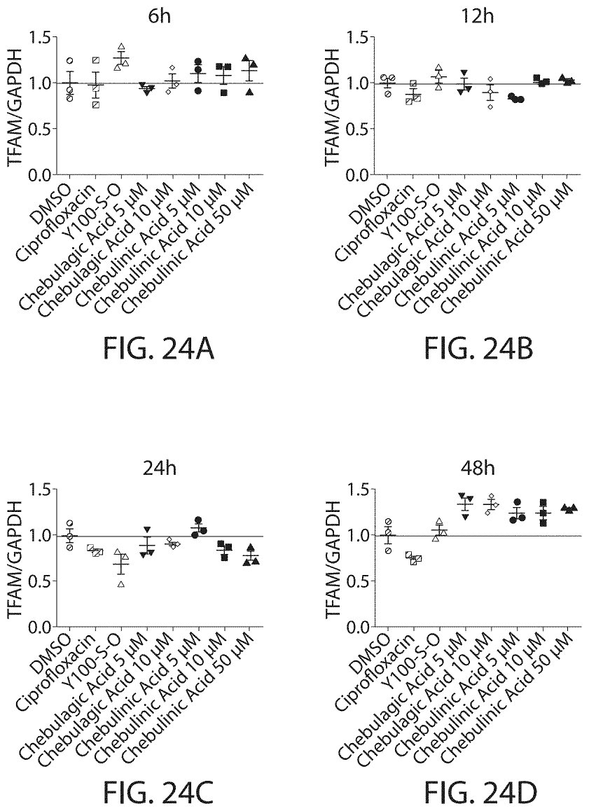

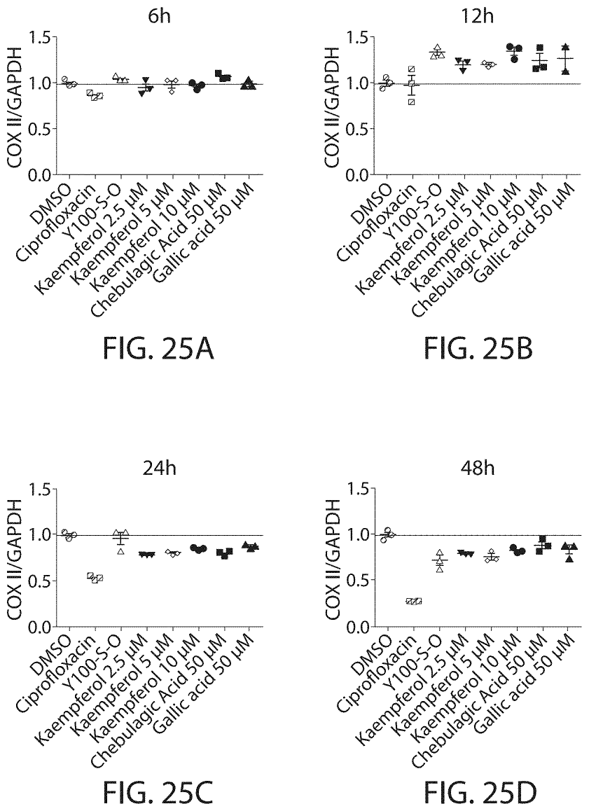

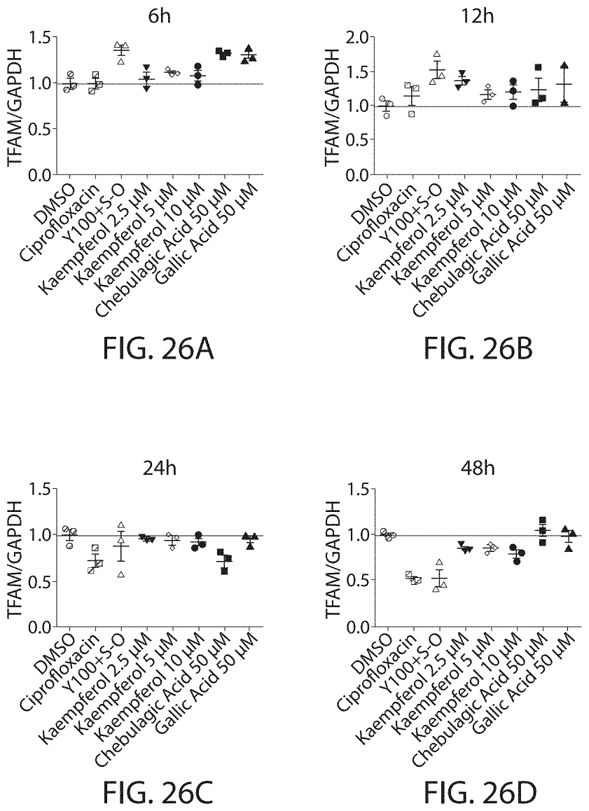

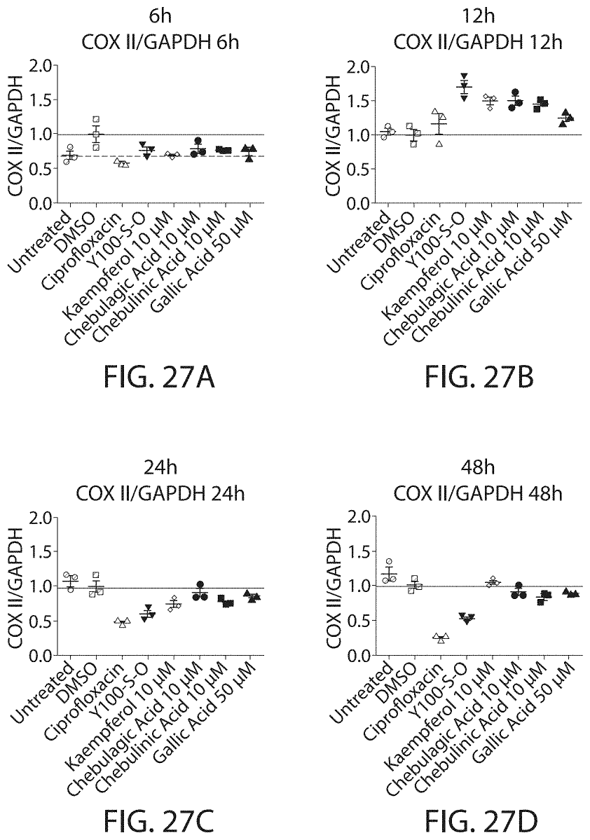

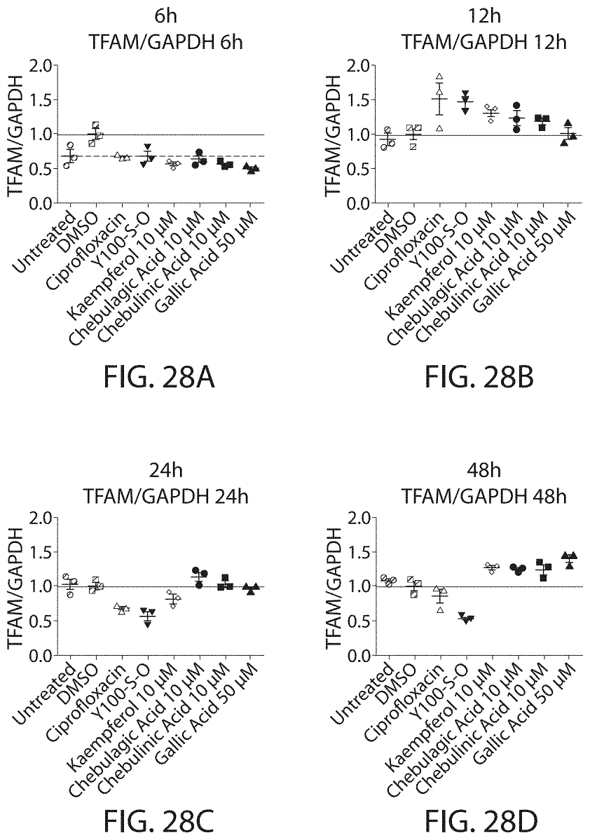

Selected constituents of emblica extract were tested in vitro for induced expression of mitochondrial biogenesis regulatory proteins including COXII and TFAM at timepoints of 6, 12, 24, and 48 hours post-administration, generally as described in previous examples. Formulations of varying concentrations were tested. The results are presented in the graphs of FIGS. 16A through 28D. Briefly, chebulagic acid, chebulinic acid, kaempferol, ellagic acid, ascorbic acid, citric acid, gallic acid, quercetin, punicalagin were tested at concentrations ranging from 2.5 μM to 100 μM for induced expression of the target proteins.

### Example 11: Nanoencapsulation of Emblica Extract and Chebulinic Acid

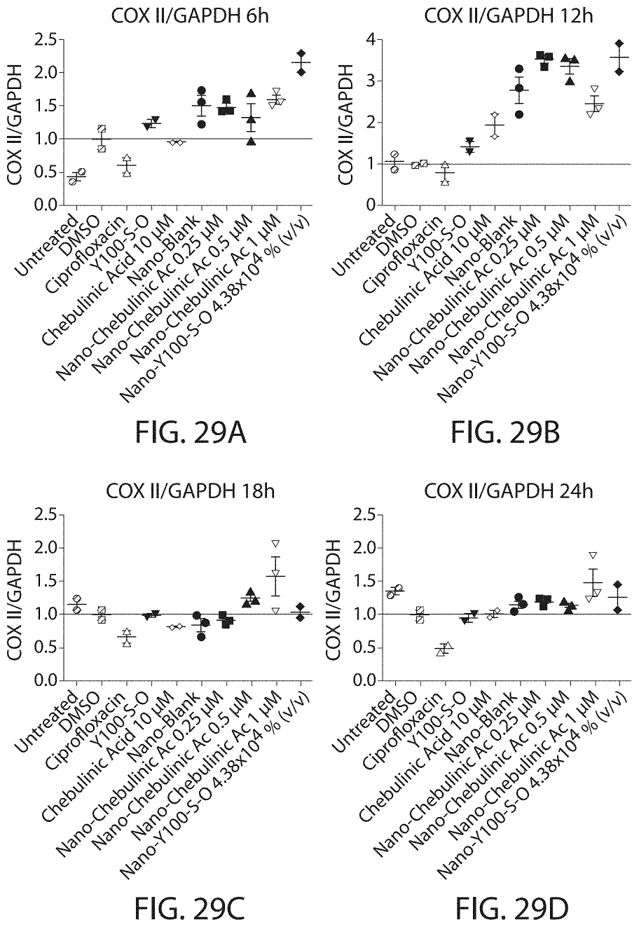

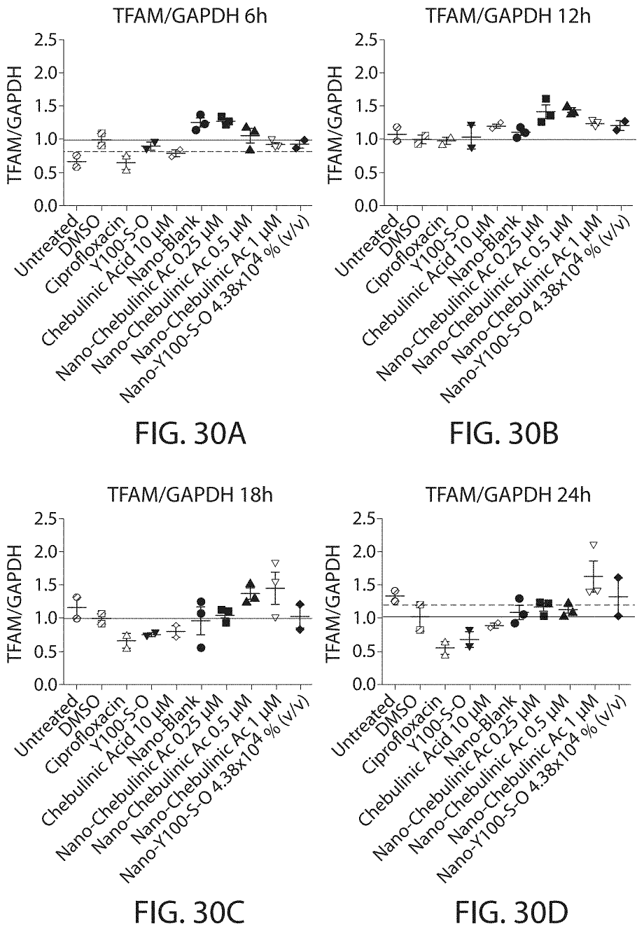

Emblica extract and chebulinic acid formulations and nanoencapsulated chebulinic acid formulations were tested in vitro for induced expression of mitochondrial biogenesis regulatory proteins COXII and TFAM at timepoints of 6, 12, 18, and 24 hours post-administration, generally as described in Example 6. Formulations of varying concentrations were tested. The results are presented in the graphs of FIGS. 29A-29D (COXII) and 30A-30D (TFAM).

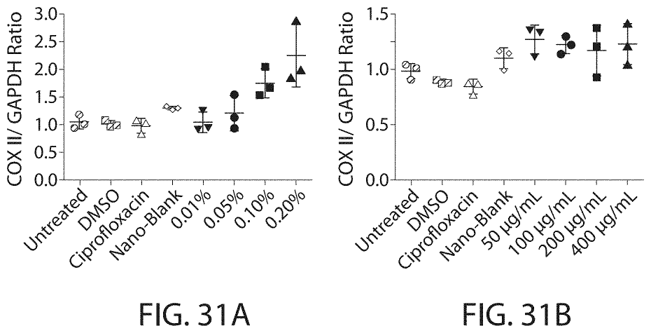

Induced expression of COXII at 6 hours post-administration of varying concentrations of nanoparticle loading from 0.01% to 0.2% emblica extract and 50 to 400 μg/L was tested. The results are presented in the graphs of FIGS. 31A-31B. As shown in the graphs of FIGS. 31A-31B, the greater effect was observed as a result of greater compound loading.

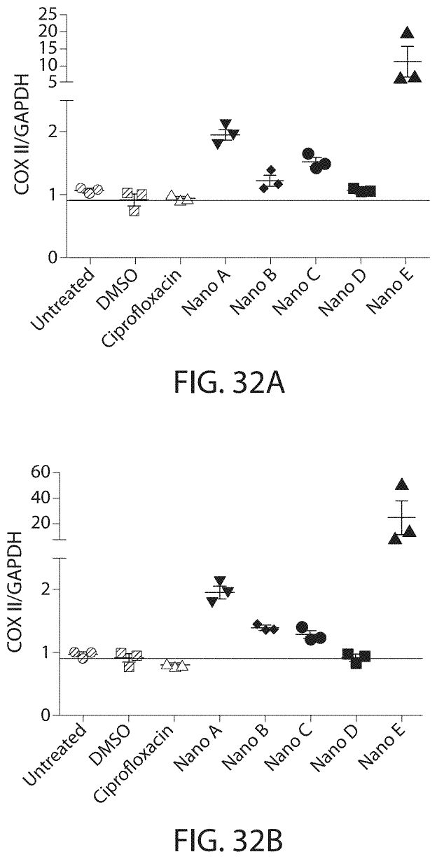

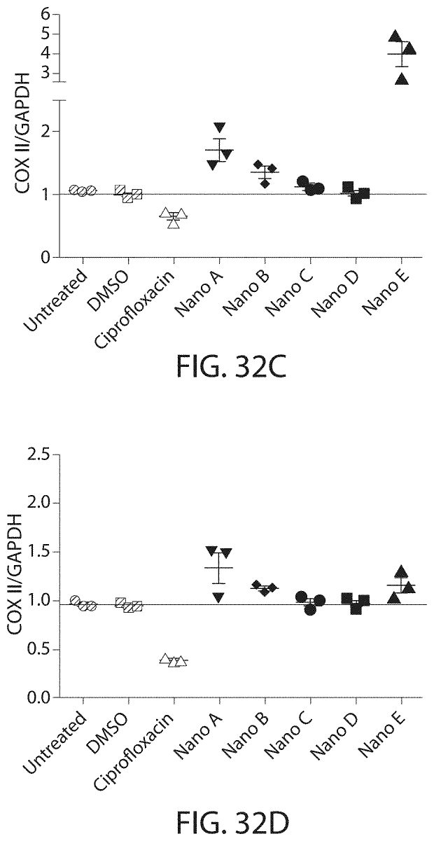

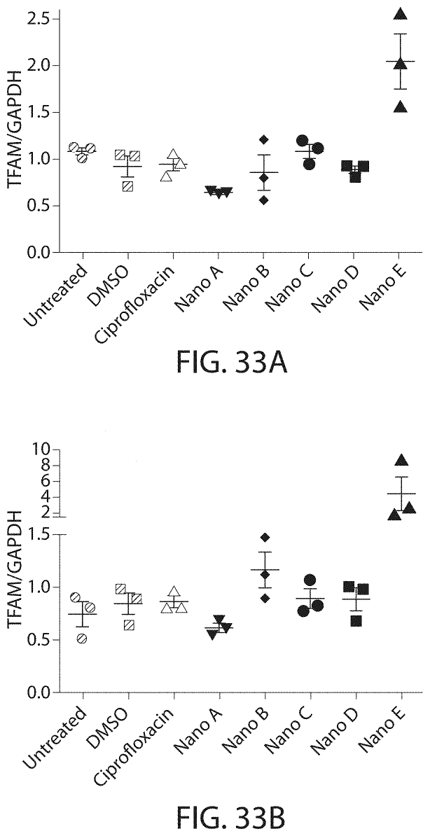

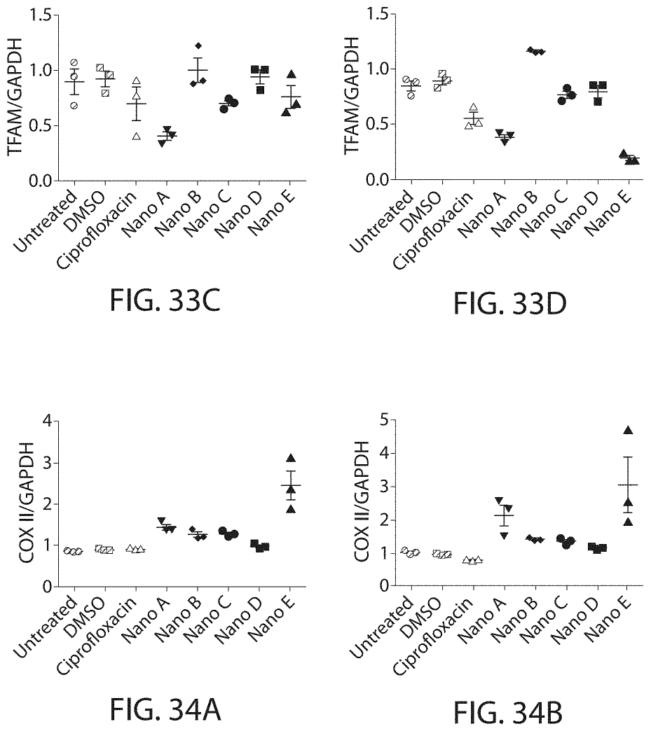

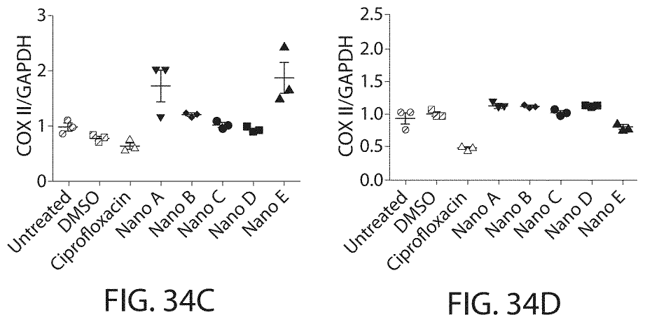

Induced expression of COXII and TFAM at 6, 12, 24, and 48 hours post-administration of nanoparticle compositions sized from 4.4 μm to 95 μm (with and without emblica extract) was tested. The results are presented in the graphs of FIGS. 32A-32D (COXII), FIGS. 33A-33D (TFAM), and FIGS. 34A-34D (COXII). In the graphs, the x axis legend is as follows:

Nano A: Y100 (emblica extract) powder loaded (40% LF; mean particle size=11.6 μm)

Nano B: Y100 (emblica extract) oil loaded (42% LF; mean particle size=9.4 μm)

Nano C: undoped blank (mean particle size=7.7 μm)

Nano D: large size octyl-doped blank (mean particle size=95 μm)

Nano E; small size octyl-doped blank (mean particle size=4.4 μm)

As shown in the graphs of FIGS. 32A-32D, 33A-33D, and 34A-34D, the greater effect was generally observed as a result of smaller particles.

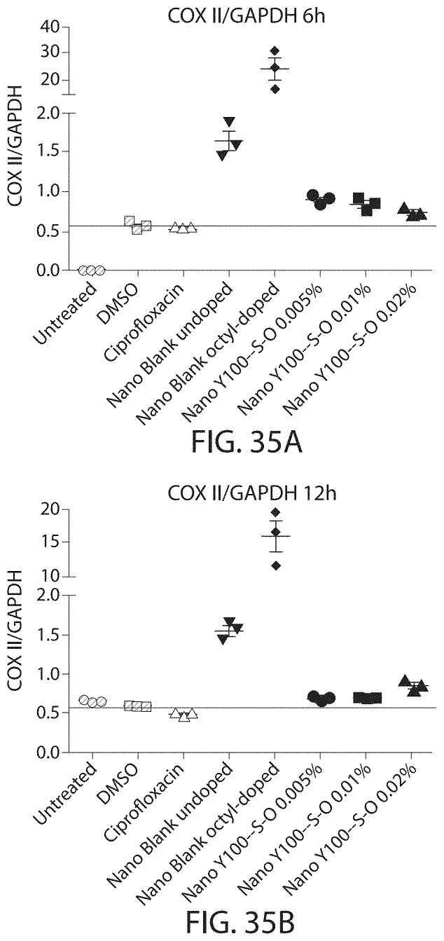

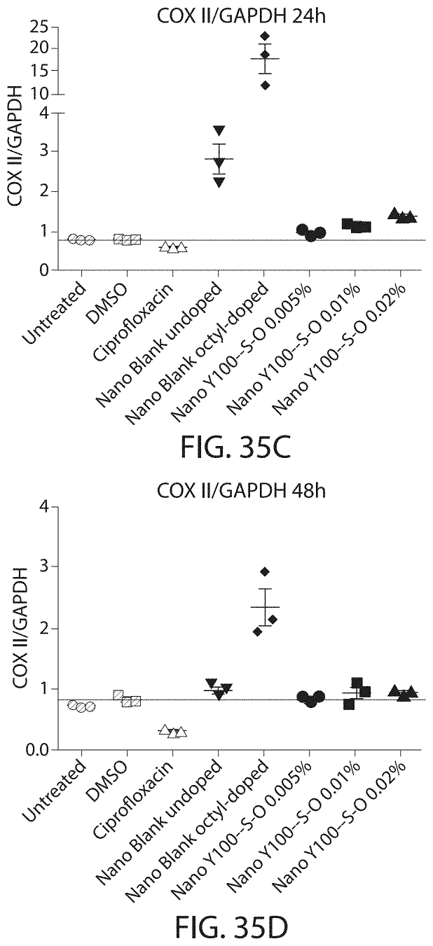

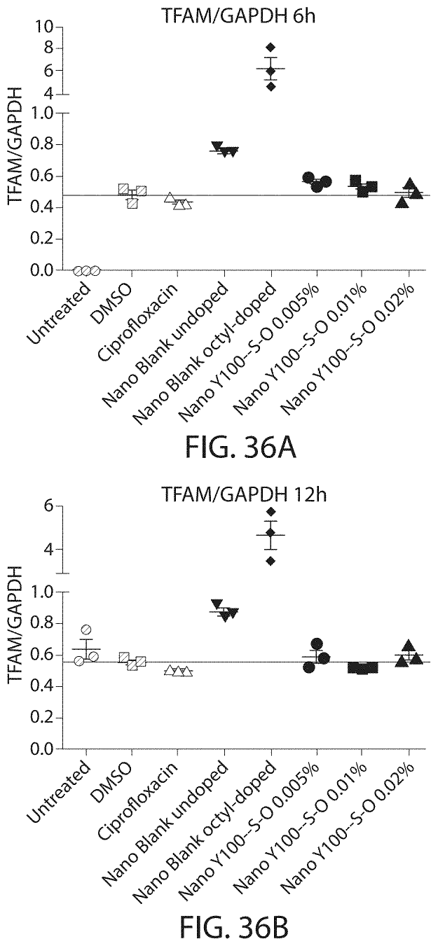

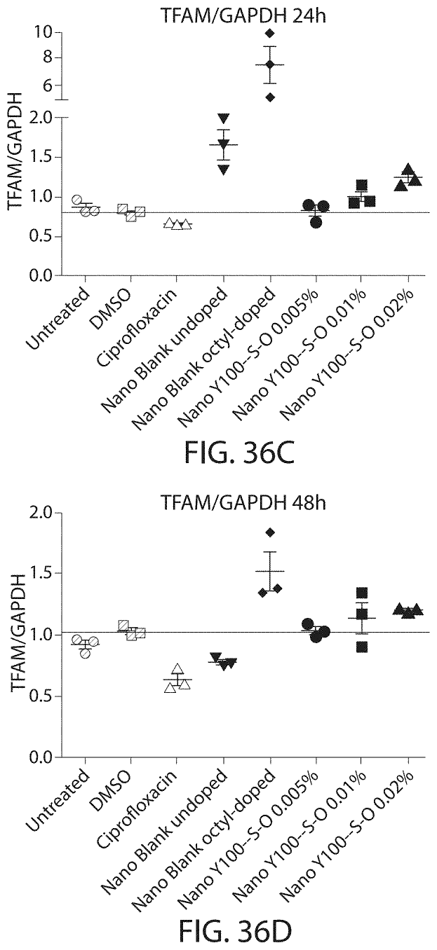

Induced expression of COXII and TFAM at 6, 12, 24, and 48 hours post-administration of nanoparticle compositions having a similar size (with and without emblica extract) (with octyl-doping and without octyl-doping) was tested. The results are presented in the graphs of FIGS. 35A-35D (COXII), and FIGS. 36A-36D (TFAM). As shown in the data presented in FIGS. 35A-35D and 36A-36D, octyl-doped particles showed a greater expression than their undoped counterparts.

Overall, the data show that induction of mitochondrial biogenesis depends on nanoparticle load and the size of nanoparticles affects mitochondrial biogenesis. Differences in nanoparticle preparation (between octyl-doped and undoped) and load have an effect on mitochondrial biogenesis induction.

### Materials and Methods

Creation of mtDNA-Depleter Mice

D1135A-POLG1 site-directed mutation was created in the full-length human POLG1 complementary DNA (cDNA) using the site-directed mutagenesis kit (Agilent, Santa Clara, Calif., USA). The primer sequences used for site-directed mutagenesis are as follows, with the mutated site in upper case: D1135A_F:5′-gcatcagcatccatgCGgaggttcgctacctgg-3′ and D1135A_R:5′-ccaggtagcgaacctcCGcatggatgctgatgc-3′. Mutations were confirmed by sequencing. D1135A-POLG1 cDNA was subcloned into the dox-inducible mammalian expression vector, pTRE-Tight-BI-AcGFP1 (Clontech, Palo Alto, Calif., USA). To obtain germline transmission of human D1135A-POLG1 (POLG1-DN), microinjection of the pTRE-Tight-BI-AcGFP1-D1135A-POLG1 construct into fertilized oocytes from C57BL/6 mouse was carried out. Potential founders were identified by screening genomic DNA from tail biopsies for the presence of the human Polg1 transgene using the PCR. The heterozygous human POLG1-positive (+/POLG1-DN⁺) founder male mice were mated with CAG-rtTA3 (rtTA) C57BL/6 female mice (Jackson Laboratories, stock no. 016532) to obtain+/POLG1-DN⁺ rtTA⁺ heterozygous transgenic mice. The +/POLG1-DN⁺ rtTA⁺ heterozygous mice were intercrossed to generate homozygous POLG1-DN⁺ rtTA⁺/POLG1-DN⁺ rtTA⁺ mice (mtDNA-depleter mice). This cross resulted in normal litter size (6-7 pups) and Mendelian distributions of genotypes, that is, 1:2:1 distribution of wild-type, heterozygous+/POLG1-DN⁺ or +/rtTA⁺ and homozygous POLG1-DN⁺ rtTA⁺/POLG1-DN⁺ rtTA⁺ showing that homozygosity for POLG1-DN allele does not result in embryonic or postnatal lethality. All the mice were given dox in diet (200 mg/kg diet) and water (2 mg/ml dox in 5% sucrose water) ad libitum. All animal experiments were conducted by following guidelines established by the Institutional Animal Care and Use Committee.

### Histological and Immunohistochemical Analyses

Skin from the dorsal side as well as other tissues was fixed in buffered formalin, embedded in paraffin, sectioned (5 μM), and stained with hematoxylin and eosin. Skin sections were stained with Giemsa stain to detect mast cells, while MPO, CD3, CD163, and Pax-5 antibodies were used for detection of other types of inflammatory cells by immunohistochemical analyses (Carson, et al., Histotechnology: A Self-Instruction Text, 3 ed., American Society for Clinical Pathology Press, Hong Kong, 2009).

RT-PCR and mtDNA Content Analyses

To measure relative gene expression by RT-PCR, total cellular RNA from the skin samples was isolated using Trizol (Invitrogen, Carlsbad, Calif., USA). Approximately, 1000-2000 ng RNA was normalized across samples, and cDNA was generated using the Iscript cDNA synthesis kit (Bio-Rad Laboratories, Hercules, Calif., USA). cDNA was then subjected to RT-PCR using Green Taq PCR mixture (Promega, Madison, Wis., USA) and gene-specific primers as given in Table 1 below. PCR products were run on 1.5 to 2% agarose gel and photographed using gel documentation system. At least three biological replicates were used in each PCR. 02-Microglobulin or RNU6B was used as an internal control in each PCR.

mtDNA content analyses in the skin and other tissues were carried out as reported earlier (Singh et al., PloS One, 10, e0139846, 2015). Briefly, the mtDNA content was analyzed by real-time PCR by absolute quantification with the following primers: mMitoF: 5′-CTAGAAACCCCGAAACCAAA-3′, mMitoR: 5′-CCAGCTATCACCAAGCTCGT-3′, mB2MF: 5′-ATGGGAAGCCGAACATACTG-3′, and mB2MR: 5′-CAGTCTCAGTGGGGGTGAAT-3′. Beta-2-Microglobulin (B2M) was used as an internal control.

TABLE 7
Primers used for genotyping

| | | |
|---|---|---|
| Target | Forward primer | Reverse primer |
| POLG1 | CAA GGT CCA GAG AGA AAC TG | CTC TGT ACC ACC CAA TTC AC |
| CAG-rtTA | CTG CTG TCC ATT CCT TAT TC | CGA AAC TCT GGT TGA CAT G |
| GFP | GGG CAA TAA GAT GGA GTA CA | TGG ACA GGT AGT GGT TAT CG |

Primers used for RT-PCR

| | | |
|---|---|---|
| POLG1 | CCA GGG AGA GTT TAT AAC CA | CAA ATT CCT CAA ACA GCC AC |
| COXII | GGC ACC TTC ACC AAA ATC AC | CGG TTG TTG ATT AGG CGT TT |
| NDI | CCT ATC ACC CTT GCC ATC AT | TTG CTG CTT CAG TTG ATC GT |
| NF-κB | TGG CCG TGG AGT ACG ACA A | GCA TCA CCC TCC AGA AGC A |
| MMP2 | ACC TGA ACA CTT TCT ATG GCT | CTT CCG CAT GGT CTC GAT G |
| | G | |
| MMP9 | CTG GAC AGC CAG ACA CTA AAG | CTC GCG GCA AGT CTT CAG AG |
| TIMP1 | CTT GGT TCC CTG GCG TAC TC | ACC TGA TCC GTC CAC AAA CAG |
| COL1A1 | CTG GCG GTT CAG GTC CAA T | TTC CAG GCA ATC CAC GAG C |
| Cyclooxygenase 2 | AAC CGC ATT GCC TCT GAA T | CAT GTT CCA GGA GGA TGG AG |
| CCL5 | AGA TCT CTG CAG CTG CCC TCA | GGA GCA CTT GCT GCT GGT GTA |
| | | G |
| IL28a | AGG TCT GGG AGA ACA TGA CTG | CTG TGG CCT GAA GCT GTG TA |
| IFNB1 | GTC ATG GGT TTC TCA TGA AGA | CAG ACC CCT TCC AGT GAT TCA |
| | ACAG | TC |
| VEGF | GAG GAT GTC CTC ACT CGG ATG | GTC GTG TTT CTG GAA GTG AGC |
| | | AA |
| IGF1R | CGA GCT TCC TGT GAA AGT GAT | CAC GTT ATG ATG ATT CGG TTC |
| | GT | TTC |
| Klotho | GGA CAT TTC CCT GTG ACT TTG | AGA GAG AGT AGT GTC CAC |
| | C | TTG AAC GT |
| MRPS5 | AAC CAC TGT CTG ACC AGC TTG | AGT CTC TGC TAA TGC GCC TTT |
| RNU6B | CTC GCT TCG GCA GCA CA | AAC GCT TCA CGA ATT TGC GT |
| B2M | ATG GGA AGC CGA ACA TAC TG | CAG TCT CAG TGG GGG TGA AT |

### Microarray Gene Analysis

Total RNA samples were extracted from POLG1 D1135A expressing MCF-7 cells after 5 days of dox induction and from the cells grown in the absence of dox for 5 days by Trizol extraction method (Invitrogen). Illumina human microarray gene expression analysis was performed with total RNA samples as described earlier.

### BN-PAGE and Western Blot Analyses

Mitochondrial isolation was carried out as previously described (Johnstone et al., J Biol Chem, 277, 42197-42204, 2002). To analyze mitochondrial OXPHOS super complexes, Blue-Native polyacrylamide gel electrophoresis (BN-PAGE) was performed with mitochondrial fractions prepared from the skin samples as described previously (Schagger et al., Methods Enzymol, 260, 190-202, 1995). Protein expression of mitochondrial OXPHOS subunits in the skin samples was carried out following standard immunoblots. A premixed cocktail containing primary monoclonal antibodies (Mitosciences, Eugene, Oreg., USA) against subunits of OXPHOS complexes was used to detect OXPHOS super complexes in BN-PAGE analyses and protein expression of OXPHOS subunits in immunoblot analyses. Voltage-dependent anion channel (VDAC) or 3-actin antibodies were used as loading controls.

### Analysis of Enzymatic Activities of OXPHOS Complexes

Isolated mitochondria were used for the measurement of enzymatic activities of OXPHOS complexes as previously described (Owens et al., PloS One, 6, e23846, 2011).

### Transmission Electron Microscopy

Transmission electron microscopic analyses of skin samples were carried as described previously (NAG et al., J Mol Cell Cardiol, 15, 301-317, 1983). Images were taken using the FEI-Tecnai electron microscope.

### Cell Culture

Skin fibroblasts from wild-type C57BL/6 (control cells) and mtDNA-depleter mice containing the D1135A-POLG1 site-directed mutation (POLG1-DN cells) were generated and spontaneously immortalized as described (Todaro et al., J Cell Biol, 17, 299-313 (1963). These cells were maintained in DMEM/F12 (Cellgro, Herndon, Va.) supplemented with 10% FBS (Atlanta Biologicals, Lawrenceville, Ga.). To induce POLG1-DN expression in skin fibroblasts, 1 μg/ml dox dissolved in water was added to the cells in culture and after 6 days of incubation, cells were washed with PBS and collected in Trizol for isolation of total RNA.

To estimate cell proliferation and cell survival, MTT assays were carried out as described previously (Ronghe et al., J Steroid Biochem Mol Biol, 144 PtB, 500-512, 2014). Both control and POLG1-DN cells were first treated with dox (1 μg/ml) for 3 days and then cells were plated at a density of 3000 cells/well in 96 well plate with or without dox (1 μg/ml) containing culture media. Readings were taken at every 24 hours.

### Preparation of Emblica Extract

Emblica officinalis was obtained from commercially available caplets (Himalaya Drug Company, Sugar Land, Tex.). Each caplet contains 600 mg (250 mg fruit extract (45% tannins), 350 mg powder stem (2% tannins). Caplets were ground and dissolved in sterile water to make 100 mg/ml solution and then filtered. The emblica solution was then mixed with an appropriate ointment base for topical application. Suitable ointment bases include, but are not limited to, dermabase ointment (MARCELLE®; water, mineral oil, propylene glycol, stearyl alcohol, cetyl esters, cetyl alcohol, glyceryl stearate, sodium lauryl sulfate, lecithin, and methylparaben) and 1:1 w/v) and Geritrex hydrophilic ointment (NDC 54162-670-14). The ointment base and emblica solution may be added at any convenient ratio, for example from 1:5 w/v emblica solution to ointment base to 5:1 w/v emblica solution to ointment base. The ointment base and emblica solution may be added at a 1:1 w/v ratio emblica solution to ointment base.

### Preparation of Fucus Extract

Fucus vesiculosus (also known as bladder wrack, black tang, rockweed, bladder fucus, sea oak, cut weed, dyers fucus, red fucus, and rock wrack) powder was obtained from Maine Coast Sea Vegetables, Inc. (Hancock, Minn.). An aqueous solution (100 mg/ml) solution was prepared from the fucus powder and filtered. The fucus solution was then mixed with an appropriate ointment base for topical application. Suitable ointment bases include, but are not limited to, dermabase ointment (MARCELLE®; water, mineral oil, propylene glycol, stearyl alcohol, cetyl esters, cetyl alcohol, glyceryl stearate, sodium lauryl sulfate, lecithin, and methylparaben) and 1:1 w/v) and Geritrex hydrophilic ointment (NDC 54162-670-14). The ointment base and fucus solution may be added at any convenient ratio, for example from 1:5 w/v fucus solution to ointment base to 5:1 w/v fucus solution to ointment base. The ointment base and fucus solution may be added at a 1:1 w/v ratio fucus solution to ointment base.

### Emblica Extract In Vivo Experimental Design

For animal experiments, the dorsal skin of the mice was depilated (for example, shaved under low-dose isoflurane inhalation anesthesia) approximately 2 days before initiation of administration of a composition to the skin. Various active compositions and control compositions were applied topically to the dorsal skin daily as described. The skin of the mice was depilated prior to collection and analysis.

Emblica extract ointment at a concentration of 100 mg/mL aqueous solution was applied topically each day to a defined shaved area of the dorsal skin of the mice. Control compositions comprised the same amount of ointment base without addition of emblica extract.

Fucus extract ointment at a concentration of 100 mg/mL aqueous solution was applied topically each day to a defined shaved area of the dorsal skin of the mice. Control compositions comprised the same amount of ointment base without addition of fucus extract.

mtDNA-depleter mice (containing the D1135A-POLG1 mutation) as well as wild-type C57BL/6 mice (control) were used in the prevention and therapeutic experiments.

In the preventive experiments, daily emblica extract treatment as described above was started 7 days before starting the dox-mediated induction of POLG1-DN. Both mtDNA-depleter and wild-type C57BL/6 mice were divided in two treatment groups: i) a control group (treated with ointment base only, n=5); and ii) a test group (treated with emblica extract ointment, n=5). Daily administration continued through the end of the experiment (up to 112 days).

In the therapeutic experiments, the wild-type C57BL/6 mice (control) and mtDNA-depleter mice were first induced with dox for 30 days and then emblica extract treatment as described above was applied daily to the end of the experiment (up to 112 days) Both mtDNA-depleter and wild-type C57BL/6 mice were divided in two treatment groups: i) a control group (treated with ointment base only, n=5); and ii) a test group (treated with emblica extract ointment, n=5).

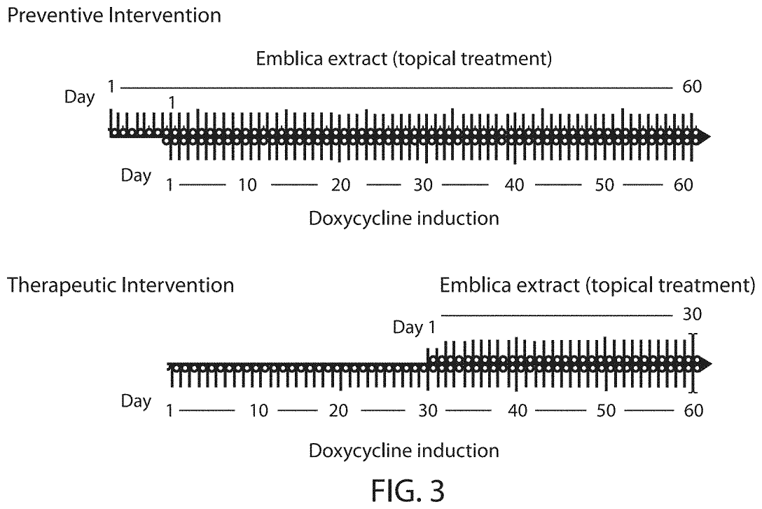

For periods of dox administration, mice were given dox in diet (200 mg/kg diet) and water (2 mg/ml dox in 5% sucrose water) ad libitum for Examples 1 to 13 and mice were given dox in diet (2 mg/kg diet) and water (2 mg/ml dox in 5% sucrose water) ad libitum for Examples 14 to 15. Schematics of both preventive and therapeutic in vivo experiments are shown in FIG. 3, noting that in certain experiments in Examples 3-5 the time course of emblica extract or fucus extract administration continued beyond the end of the time periods specified in FIG. 3 (i.e., up to 112 days).

### Statistical Analyses

Statistical analyses were performed using unpaired Student's t test. Data are expressed as mean±s.e.m. P values<0.05 were considered significant. All cellular experiments were repeated at least three times.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. A method of treating or preventing viral infection-induced symptoms in a subject, the method comprising administering to the subject a composition comprising an effective amount of one or more of an emblica extract, a fucus extract, and a chebula extract, or a pharmaceutically acceptable form thereof, or one or more compounds constituent of one or more of an emblica extract, a fucus extract, and a chebula extract, or a pharmaceutically acceptable form thereof, or one or more compounds having a similarity score of at least 95% with a compound constituent of one or more of an emblica extract, a fucus extract, and a chebula extract, or a pharmaceutically acceptable form thereof.

2. A method of treating or preventing a viral infection in a subject, the method comprising administering to the subject a composition comprising an effective amount of one or more of an emblica extract, a fucus extract, and a chebula extract, or a pharmaceutically acceptable form thereof, or one or more compounds constituent of one or more of an emblica extract, a fucus extract, and a chebula extract, or a pharmaceutically acceptable form thereof, or one or more compounds having a similarity score of at least 95% with a compound constituent of one or more of an emblica extract, a fucus extract, and a chebula extract, or a pharmaceutically acceptable form thereof.

3. A method of treating or preventing mitochondrial dysfunction in a subject, the method comprising administering to the subject a composition comprising an effective amount of one or more of an emblica extract, a fucus extract, and a chebula extract, or a pharmaceutically acceptable form thereof, or one or more compounds constituent of one or more of an emblica extract, a fucus extract, and a chebula extract, or a pharmaceutically acceptable form thereof, or one or more compounds having a similarity score of at least 95% with a compound constituent of one or more of an emblica extract, a fucus extract, and a chebula extract, or a pharmaceutically acceptable form thereof.

4. The method of claim 1, comprising administering to the subject an effective amount of an emblica extract, one or more compound constituent of an emblica extract or having a similarity score of at least 95% with a compound constituent of an emblica extract, or a pharmaceutically acceptable form thereof.

5. The method of claim 1, comprising administering to the subject an effective amount of a fucus extract, one or more compound constituent of a fucus extract or having a similarity score of at least 95% with a compound constituent of a fucus extract, or a pharmaceutically acceptable form thereof.

6. The method of claim 1, comprising administering to the subject an effective amount of a chebula extract, one or more compound constituent of a chebula extract or having a similarity score of at least 95% with a compound constituent of a chebula extract, or a pharmaceutically acceptable form thereof.

7. The method of claim 1, wherein the emblica extract is derived from Emblica officinalis.

8. The method of claim 1, wherein the compound constituent of the emblica extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a-C(O)OH, wherein a is 0 to 5, and 1 to 5 hydroxy groups, or a pharmaceutically acceptable form thereof, or a combination of the foregoing.

9. The method of claim 1, wherein the compound constituent of the emblica extract is gallic acid, vanillic acid, chlorogenic acid, 5 caffeic acid, syringic acid, coumaric acid, quercetin, emblicanin A, emblicanin B, punigluconin, and pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

10. The method of claim 1, wherein the fucus extract is derived from Fucus vesiculosus, Fucus serratus, Fucus, spiralis, or Fucus guiryi.

11. The method of claim 1, wherein the compound constituent of the fucus extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a-C(O)OH, wherein a is 0 to 5, and 1 to 5 hydroxy groups, or a pharmaceutically acceptable form thereof, or a combination of the foregoing.

12. The method of claim 1, wherein the compound constituent of the fucus extract is gallic acid, vanillic acid, chlorogenic acid, caffeic acid, syringic acid, coumaric acid, quercetin, fucoidan, punigluconin, and pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

13. The method of claim 1, wherein the chebula extract is derived from Terminilia chebula, Terminalia arborea, or Lumnitzera racemose.

14. The method of any of claim 1, wherein the compound constituent of the chebula extract is a benzoic acid substituted with 1 to 5 hydroxy groups and optionally 1 to 3 O—(C1-C5 alkyl) or O—(C1-C5 alkenyl) groups, or a pharmaceutically acceptable form thereof, or a benzene substituted with —CH═CH—(CH₂)a —C(O)OH, wherein a is 0 to 5, and 1 to 5 hydroxy groups, or a pharmaceutically acceptable form thereof, or a combination of the foregoing.

15. The method of claim 1, wherein the compound constituent of the chebula extract is gallic acid, vanillic acid, chlorogenic acid, caffeic acid, syringic acid, coumaric acid, quercetin, fucoidan, punigluconin, and pedunculagin, punicafolin, phyllanemblin, kaempferol, ellagic acid, chebulinic acid, chebulagic acid, punicalagin, a metabolite of any of the foregoing, a compound having a similarity score of at least 95% with any of the foregoing, or a pharmaceutically acceptable form of any of the foregoing.

16. The method of claim 1, wherein the composition comprises two or more of: an effective amount of an emblica extract, or a pharmaceutically acceptable form thereof, or a compound constituent of an emblica extract, or a pharmaceutically acceptable form thereof, or a compound having a similarity score of at least 95% with a compound constituent of an emblica extract, or a pharmaceutically acceptable form thereof; an effective amount of a fucus extract, or a pharmaceutically acceptable form thereof, or a compound constituent of a fucus extract, or a pharmaceutically acceptable form thereof, or a compound having a similarity score of at least 95% with a compound constituent of a fucus extract, or a pharmaceutically acceptable form thereof; and an effective amount of a chebula extract, or a pharmaceutically acceptable form thereof, or a compound constituent of a chebula extract, or a pharmaceutically acceptable form thereof, or a compound having a similarity score of at least 95% with a compound constituent of a chebula extract, or a pharmaceutically acceptable form thereof.

17. The method of claim 1, wherein the composition is fortified with one or more compounds constituent of an emblica extract, or a pharmaceutically acceptable form thereof, or a compound having a similarity score of at least 95% with a compound constituent of an emblica extract, or a pharmaceutically acceptable form thereof.

18. The method of claim 1, wherein the composition is fortified with one or more compounds constituent of a fucus extract, or a pharmaceutically acceptable form thereof, or a compound having a similarity score of at least 95% with a compound constituent of a fucus extract, or a pharmaceutically acceptable form thereof.

19. The method of claim 1, wherein the composition is fortified with one or more compounds constituent of a chebula extract, or a pharmaceutically acceptable form thereof, or a compound having a similarity score of at least 95% with a compound constituent of a chebula extract, or a pharmaceutically acceptable form thereof.

20. The method of claim 1, wherein the one or more compounds constituent of the emblica extract or having a similarity score of at least 95% with a compound constituent of the emblica extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

21. The method of claim 1, wherein the one or more compounds constituent of the fucus extract or having a similarity score of at least 95% with a compound constituent of the fucus extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

22. The method of claim 1, wherein the one or more compounds constituent of the chebula extract or having a similarity score of at least 95% with a compound constituent of the chebula extract is purified, e.g., at least 80% purified, at least 85% purified, at least 90% purified, at least 95% purified, at least 98% purified, at least 99% purified, at least 99.9% purified, at least 99.99% purified, or at least 99.999% purified.

23. The method of claim 1, wherein the viral infection-induced symptoms in the subject comprise one or more acute and/or chronic symptoms, e.g., muscle or body aches, fatigue, shortness of breath, difficulty breathing, fever or chills, headache, loss of taste or smell, sore throat, congestion or runny nose, nausea or vomiting, diarrhea, cough, lymphadenitis, rash, or sleep hyperhidrosis.

24. The method of claim 2, wherein the viral infection in the subject is SARS, e.g., SARS-CoV-1 or SARS-CoV-2.

25. The method of claim 2, wherein the viral infection in the subject is HIV.

26. The method of any claim 2, wherein the viral infection in the subject is Influenza.

27. The method of claim 2, wherein the viral infection in the subject is MERS.

28. The method of claim 2, wherein the viral infection in the subject is any viral infection related to mitochondrial dysfunction in the subject.

29. The method of claim 1, wherein the effective amount is a therapeutically effective amount.

30. The method of claim 3, wherein the treatment or prevention involves inducing mitochondrial biogenesis and/or improving mitochondrial function.

31. The method of claim 3, wherein the effective amount or therapeutically effective amount is sufficient to induce mitochondrial biogenesis.

32. The method of claim 1, wherein administration increases expression of at least one protein selected from PGC-1a, TFAM, NRF-1, and COX II.

33. The method of claim 1, wherein administration decreases expression or inhibits an increase of expression of at least one protein selected from FGF-21 and IL-6, or any cytokine related to viral infection.

34. The method of claim 1, wherein administration increases or inhibits a decrease in at least one of ATP-linked respiration, maximal respiration and reserve capacity in subjects infected with SARS-CoV-2.

35. The method of claim 2, wherein administration decreases the release of viral vesicles from dysfunctional mitochondria.

36. The method of claim 2, wherein administration modulates, e.g., increases or decreases, viral protein interaction with one or more host mitochondrial genes, e.g., MRPS2, MRPS5, MRPS25, MRPS27, NDUFAF1, NDUFB9, NDUFAF2, ATP1B1, ATP6V1A, ACADM, AASS, PMPCB, PITRM1, COQ8B, PMPCA, and Tomm70.

37. The method of claim 1, wherein administration decreases or inhibits an increase in circulating mtDNA levels, e.g., plasma mtDNA and cytoplasmic mtDNA.

38. The method of claim 1, wherein the composition is administered topically.

39. The method of claim 1, wherein the composition is administered parenterally, e.g., intravenously, intraperitoneally, or intramuscularly.

40. The method of claim 1, wherein the composition is administered enterally.

41. The method of claim 1, wherein the composition is formulated as a topical solution, oil, cream, emulsion, or gel.

42. The method of claim 1, wherein the composition is formulated as a shampoo, conditioner, spray, cream, gel, balm, body wash, soap, lotion, or make-up.

43. The method of claim 1, wherein the composition is formulated as a parenteral liquid solution.

44. The method of claim 1, wherein the composition is formulated as an enteral capsule or tablet, or dietary supplement or food, e.g., food, food supplement, medical food, food additive, nutraceutical, or drink.

45. The method of claim 1, wherein the composition is administered locally.

46. The method of claim 1, wherein the composition is administered systemically.

47. The method of claim 1, wherein the composition is formulated for immediate release.

48. The method of claim 1, wherein the composition is formulated for extended release, e.g., controlled or sustained release.

49. The method of claim 2, wherein the composition is administered in combination with standard of care treatment for one or more of SARS-CoV-2, HIV, SARS-CoV-2, HIV, Influenza, MERS, or any viral infection related to mitochondrial dysfunction in a subject.

50. The method of claim 1, wherein the composition is administered in combination with one or more drugs for symptomatic relief, e.g., acetaminophen, ibuprofen, bismuth subsalicylate, loperamide, oxymetazoline, phylephrine, psudoephedrine, or hydrocortisone.

51. The method of claim 2, wherein the composition is administered in combination with one or more anti-viral drugs, e.g., remdesivir, abacavir, didanosine, emtricitabine, iamivudine, stavudine, zalcitabine, zidovudine, tenofovir disproval fumarate, peramivir, zanamivir, oseltamivir phosphate, baloxavir marboxil, ribavirin, interferon-α, lopinavir/ritonavir, and convalescent plasma.

52. The method of claim 1, wherein the composition comprises a nanoparticle-based delivery carrier.

53. The method of claim 1, wherein the composition comprises a skin penetration enhancer or is administered in combination with a skin penetration enhancer, e.g., a chemical skin penetration enhancer or a physical skin penetration enhancer.

54. The method of claim 3, wherein the composition comprises a mitochondria-targeting agent or a delivery carrier functionalized with a mitochondria-targeting agent.

55. The method of claim 1, wherein the compound constituent of an emblica extract was derived from, purified from, or isolated from the emblica extract.

56. The method of claim 1, wherein the compound constituent of an emblica extract was derived from, purified from, or isolated from a source other than the emblica extract.

57. The method of claim 1, wherein the compound constituent of an emblica extract was synthesized.

58. The method of claim 1, wherein the compound constituent of a fucus extract was derived from, purified from, or isolated from the fucus extract.

59. The method of claim 1, wherein the compound constituent of a fucus extract was derived from, purified from, or isolated from a source other than the fucus extract.

60. The method of claim 1, wherein the compound constituent of a fucus extract was synthesized.

61. The method of claim 1, wherein the compound constituent of a chebula extract was derived from, purified from, or isolated from the chebula extract.

62. The method of claim 1, wherein the compound constituent of a chebula extract was derived from, purified from, or isolated from a source other than the chebula extract.

63. The method of claim 1, wherein the compound constituent of a chebula extract was synthesized.
